(19) United States
(12) Patent Application Publication
(10) Pub. No.: US 2025/0283055 A1
(43) Pub. Date: Sep. 11, 2025

(54) BROAD SPECTRUM VIRUS-TRAPPING NANOSHELLS

(71) Applicant: TECHNISCHE UNIVERSITÄT MÜNCHEN, München (DE)

(72) Inventors: Alba MONFERRER, München (DE); Jessica KRETZMANN, Unterföhring (DE); Hendrik DIETZ, Haar (DE)

(21) Appl. No.: 18/859,607

(22) PCT Filed: Apr. 28, 2023
(86) PCT No.: PCT/EP2023/061264

§ 371 Date: Oct. 24, 2024

(30) Foreign Application Priority Data
Apr. 28, 2022 (EP) 22170578.3

(51) Int. Cl. C12N 7/04; C12N 15/11

(52) U.S. Cl. CPC C12N 7/04; C12N 15/11

(57) ABSTRACT

The present invention relates to a DNA-based nanostructure for encapsulating viruses or viral particles, to a composition comprising one or more viruses or viral particles encapsulated by such a DNA-based nanostructure according to the present invention, and to a method for encapsulating one or more viruses or viral particles by using such a DNA-based nanostructure.

### RELATED APPLICATIONS

This application is the U.S. national phase of International Patent Application No. PCT/EP2023/061264, filed Apr. 28, 2023, which claims priority to European Patent Application No. 22170578.3, filed Apr. 28, 2022, which are hereby incorporated by reference in their entirety.

### INCORPORATION BY REFERENCE OF SEQUENCE LISTING

This application includes, as a separate part of disclosure, a Sequence Listing in computer-readable form (Filename: SubSequenceListing.xml, 2,370,592 bytes; created Mar. 20, 2025). The contents of the Sequence Listing xml file are incorporated herein by reference in its entirety.

### FIELD OF THE INVENTION

The present invention relates to a DNA-based nanostructure for encapsulating a broad spectrum of viruses or viral particles, to a composition comprising one or more viruses or viral particles encapsulated by such a DNA-based nanostructure according to the present invention, and to a method for encapsulating one or more viruses or viral particles by using such a DNA-based nanostructure.

### BACKGROUND OF THE INVENTION

At present, there are over 200 known viral-vector borne human diseases, of which only nine are treatable with current antiviral drugs (Heida et al., Drug Discov. Today 26 (2021) 122-137). In the search for effective antiviral therapies, neutralizing antibodies are increasingly being considered for treating acute viral infections (see, for example, Wang et al., Science. 2021 Aug. 13; 373 (6556): eabh1766. doi: 10.1126/science.abh1766. Epub 2021 Jul. 1. PMID: 34210892; Taylor, P. C. et al., Nat. Rev. Immunol. 21 (2021) 382-393). Antiviral antibodies often derive their virus-neutralizing function by blocking the interactions that viruses undergo with specific receptors on the surface of host cells that are required for receptor-mediated cell invasion. However, antibodies are prone to losing their function due to mutational drift, take time to develop, and will only be effective for one virus or virus serotype at a time. Furthermore, antibodies or other proteinaceous virus binders may cause adverse immunogenic effects in organisms and create substantial additional fabrication hurdles and costs.

Recently, a new concept for neutralizing viruses by encapsulation in macromolecular shells fabricated with DNA origami has been presented (WO 2021/165528). The shells mechanically prevent interactions between trapped viruses and host cells. For the proof-of-concept experiments, the inside of the shell was coated with antibodies to sequester virus particles in the shells. Heparin is mentioned as a potential alternative binding moiety, but no data are shown. One key advantage of the shells is that the virus-binding moieties used in their interior themselves do not need to have a neutralizing function, since this task is performed by the shell material. Nonetheless, as stated above, the use of antibodies in the virus trapping shells presents several challenges that may limit the usefulness of the virus-trapping concept. WO 2021/165528 used up to 90 antibodies per virus-trapping shells, which were attached via interaction with single-stranded oligonucleotide handles with 16-mer or 26-mer overhangs for hybridization. WO 2021/165528 demonstrates that virus particles can successfully be encapsulated in shells equipped with antibodies, but does not quantify the rate of encapsulations being achieved.

The concept laid out in WO 2021/165528 was described in a scientific publication as well (Sigl et al., Nat. Mater. 20 (2021) 1281-1289). Sigl et al. show the successful encapsulation of virus particles using antibody-equipped virus particles. Heparin is not mentioned as an alternative binding moiety, and no quantification of the rate of encapsulations is provided.

Knappe et al. (ACS Nano 2021; 14316-14322) describe DNA origami particles that are functionalized via click chemistry, so that different types of functional moieties, including antibodies and carbohydrates, can be coupled to the DNA origami particles. Heparin is not specifically mentioned in Knappe et al., and functionalization is performed at the outside of the DNA origami particles, since the DNA origami particles are closed shells. Thus, no information can be derived from Knappe et al. about the options for encapsulating virus particles in the interior of DNA origami shells and about any particularities of the optimal design of the attachment sites.

Another attractive avenue is the packaging of viral payload for the purposes of delivering viruses or other vectors for genetic information or other cargo to target cells or target tissue, as discussed e.g., in Antigen-Triggered Logic-Gating of DNA Nanodevices, Engelen et al., J. Am. Chem. Soc. 2021, 143, 51, 21630-21636, Dec. 20, 2021. Also here the use of antibodies as moieties to attach viruses or virus-like particles or other assemblies within DNA-based shells may limit the scope of such applications, due to the challenges listed above arising from the use of antibodies.

Thus, while different strategies for the treatment of viral infections have been developed or suggested up to date, there is still a need for the development of a concept of a generic antiviral drug platform for targeting a variety of viral pathogens. In particular, a concept would be highly desirous that does not rely on prior detailed knowledge about genetics and properties of the target virus. Additionally, there is an unmet need for the development of a system that permits the encapsulation of virus particles with high efficiency.

### SUMMARY OF THE INVENTION

It is an object of the present invention to provide constructs that enable the encapsulation of one or more viruses or viral particles. The solution to that problem, i. e. the use of macromolecular building blocks, such as DNA-based nanostructures, has not yet been taught or suggested by the prior art.

Therefore, in one aspect, the present invention relates to a DNA-based nanostructure, wherein said DNA-based nanostructure is a shell comprising a cavity enclosed by said DNA-based nanostructure, wherein said DNA-based nanostructure is formed by self-assembling DNA-based building blocks, wherein each of said self-assembling DNA-based building blocks is formed by a single-stranded DNA template strand and a set of oligonucleotides complementary to said single-stranded DNA template, wherein each of said oligonucleotides is either complementary to one contiguous DNA sequence stretch or to at least two non-contiguous DNA sequence stretches on said single-stranded DNA template, wherein each of said self-assembling DNA-based building blocks is a triangular and/or a rectangular prismoid, particularly a triangular prismoid, and wherein a subset of one or more of said oligonucleotides in one or more of said self-assembling DNA-based building blocks is/are each linked to a construct comprising at least one sulfonated or sulfated polysaccharide group pointing to the interior of said cavity, particularly a construct comprising one or two sulfonated or sulfated polysaccharide groups, wherein each said construct comprises (i) a handle comprising at least one binding site for said sulfonated or sulfated polysaccharide group, and (ii) said sulfonated or sulfated polysaccharide group(s) bound to said handle; wherein said handle has a length corresponding to at least the length of a single-stranded oligonucleotide comprising 30 nucleotides.

In a second aspect, the present invention relates to a composition comprising a DNA-based nanostructure according to the present invention encapsulating one or more viruses or viral particles.

In a third aspect, the present invention relates to a method for encapsulating one or more viruses or viral particles, comprising the steps of: providing a DNA-based nanostructure according to the present invention, and contacting said DNA-based nanostructure with a medium comprising, or suspected to comprise, said viruses or viral particles.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIG. 1 shows the DNA origami shells and functionalization with HS derivatives. A: A heparan sulfate proteoglycan (HSPG) interacts with a virus pathogen and mediates its cellular uptake (left). DNA origami shell schematics, with HS modifications in its interior, capable of binding and sequestering a viral particle (right). B: SPAAC reaction in between azide-modified HS oligomers and a DBCO-modified DNA oligo. The DNA sequence is complementary to the handles of the DNA origami shells in (e,f). C: PAGE characterization of the HS-modified DNA oligos. Products containing HS with sulfate and sulfonate groups (3a and 3c) migrate at a faster rate through the gel than the analog negative controls (3b and 3d) due to their increased anionic character. D: Cylindrical models of O and T1 shells made of 4 and 10 triangle subunits respectively, containing single stranded protruding oligos (termed handles, shown in red) decorating their interior. Each triangle subunit contains 9 handle positions. E: New T3 shell design consisting of 30 triangle subunits and featuring an inner cavity of 150 nm. Each triangle subunit also contains 9 handle positions. F: Negative stain TEM micrograph of T3 shells. Scale bar is 100 nm. G: Schematic representation of three different handle designs. H1 contains one HS modification per handle placed as close to the origami surface as possible. H2 also contains one HS modification per handle but has a polyT extension of 20 bases, allowing the handle to reach further than H1. H3 mimics a branched polymer containing two HS modifications per handle unit, therefore doubling the local HS density.

FIG. 2 shows viruses and VLPs trapped within HS-modified O, T1 and T3 shells. Negative stain TEM captions of a: AAV2, polio 3, mature dengue 1 and norovirus Gll.4 successfully trapped in O shells; b: HPV 16, SARS-CoV-2, chikungunya and rubella engulfed by T1 shells; c: adenovirus 5 captured with T3 shells. Scale bars are 100 nm.

FIG. 3 shows multiple viruses and VLPs trapped in HS-modified O, T1 and T3 shells Negative stain TEM images of a: up to four AAV2 in one O shell, b: up to three HPV 16 in one T1 shell, c: one HPV 16 coordinated by two O shells for complete occlusion of the virus particle; d: up to six AAV2 per T1 shell; e: up to three chikungunya VLPs per T3 shell; f: cooperative effect of multiple O shells capturing numerous AAV2 particles. Scale bar is 100 nm.

FIG. 4 shows cryo-EM analysis of virus-like particles trapped in DNA origami shells. a: Cryo-EM micrograph of O shells binding to HPV 16 VLPs; b: 2D class average images of one or two O shells binding to one HPV 16 particle, demonstrating different orientations of the complexes. The white arrows indicate the gap difference in between the two O shells, confirming the capture of differently sized VLP particles; c: 3D reconstructions of HPV 16 bound to one and two O shells; d: Cryo-EM micrograph of T1 shells binding to chikungunya VLPs; e: 2D class average images of T1 shells binding to chikungunya particles showing different orientations of the complex; f: Two different views of the 3D reconstruction of a T1 shell engulfing a chikungunya virus particle.

FIG. 5 shows the SPAAC reaction for 8-mer HS derivatives (1a and 1b). a: Click chemistry reaction in between azide-modified HS polymers and a DBCO-modified DNA oligo. The DNA sequence is complementary to the handles of the DNA origami shells. b: PAGE characterization of all the HS-modified DNA oligos.

FIG. 6 shows T3 shell design. a: Top and front view of a T3 cylindrical model. b: Cylindrical models of the triangles t1-t6 involved in the T3 shell assembly. Arrows indicate complementary side interactions.

FIG. 7 shows TEM field of view of T3 shells. The T3 DNA origami shells presented an inner diameter of ˜150 nm. Due to their flexibility, they appear deformed on the grid. Scale bar is 400 nm.

FIG. 8 shows TEM quantification of O shells for AAV2 trapping with the different handle designs. a: Schematic representation of the three different handle designs H1, H2 and H3. HS represented as red hexagons. H1 contains one HS modification per handle, placed closely to the origami surface. H2 also contains one HS modification per handle but has a polyT extension of 20 bases, allowing the handle to reach further than H1. H3 mimics a branched polymer containing two HS modifications per handle unit, therefore doubling the local HS density. b: Blind TEM quantification of full vs. empty O shells of each handle design when functionalized with 3c HS derivative and AAV2 excess. H1 presented ˜20% of full shells, H2˜84%, and H3˜96%. c: Schematic representation of an O half-shell and its ssDNA handles in the inner cavity.

FIG. 9 shows TEM of negative control for AAV2 trapping in O shells. Two fields of view of the same sample showing that no binding was observed when the 3d negative control HS modification was hybridized to H3 handle design. Scale bar is 100 nm.

FIG. 10 shows TEM of AAV2 trapping with O shell excess. Two fields of view of the same sample showing that all AAV2 particles were encapsulated when the origami shell was used in excess. Scale bar is 100 nm.

FIG. 11 shows TEM of free viruses and VLPs. TEM data showed that AAV2, poliovirus, HPV 16, chikungunya and adenovirus 5 are the purest samples of our library. Dengue, norovirus, SARS-CoV-2 and rubella visibly contained a high amount of protein debris and presented a variable range of particle sizes. Scale bar is 100 nm.

FIG. 12 shows TEM tomography of adenovirus 5 in a T3 shell. The slices of the tomogram calculated from an EM tilt series proved the full encapsulation of an adenovirus in the selected shell particle. Scale bar is 100 nm.

FIG. 13 shows TEM tomography of chikungunya VLPs in a T3 shell. The slices of the tomogram calculated from an EM tilt series proved the full encapsulation of three chikungunya VLPs in the selected shell particle. The last image slice showed a disruption of the triangles' connectivity. It was not clear if this discontinuity was due to the shell's rearrangement to encapsulate multiple VLPs or a consequence deforming on the grid during the sample preparation. Scale bar is 100 nm.

FIG. 14 shows TEM quantification of T1 shells trapping chikungunya VLPs with the 3a and 3b HS derivatives. a: negative stain TEM micrograph of T1 shells functionalized with the 3b negative control HS derivative. Due to the size and shape complementarity, weak electrostatic interactions in between the DNA and the chikungunya VLP were sufficient to keep the virus particles encapsulated when the negative control handles were used. Scale bar is 100 nm. b: TEM quantification of full vs. empty T1 shells functionalized with the 3a HS derivative on a H1 handle design. ˜90% of shells were full c: TEM quantification of full vs. empty T1 shells functionalized with the 3b negative control HS derivative on a H1 handle design. ˜54% of shells were full.

FIG. 15 shows TEM of immature and mature dengue 1 VLPs trapping with O shells. a: The immature configuration of the dengue VLPs showed no binding to the HS-modified origami shells. b: Mature dengue VLPs were recognized and encapsulated by the O shells. VLPs were used in excess. Scale bar is 100 nm.

FIG. 16 shows Cryo-EM imaging of O shells trapping HPV 16 particles (EMD-13884). a: Exemplary micrograph of O shells trapping HPV 16 vitrified on lacey carbon grids with ultrathin carbon support. b: 2D class averages of empty shells (left), HPV 16 trapped by one O shell (middle), and two O shells trapping an HPV 16 (right). c: 3D classes of selected particles showing similar particles as in b. d: 3D reconstruction of HPV 16 particles trapped in one O shell. e: Multibody refinement of HPV 16 particles encapsulated by two O shells. f+g: Multi-component analysis of two O shells trapping an HPV 16.

FIG. 17 shows Cryo-EM imaging of T1 shells trapping chikungunya VLPs (EMD-13883). a: Exemplary micrograph of T1 shells trapping chikungunya VLPs vitrified on lacey carbon grids with ultrathin carbon support. b: 2D class averages of extracted particles. c: FSC estimation of the reconstruction shown in e (C5). d: 3D classification of extracted particles. e: 3D reconstruction of T1 shells trapping chikungunya of selected particles from multiple rounds of 3D classification without (C1) and with symmetry (C5).

FIG. 18 shows 2D class averages of free HPV 16 VLPs extracted from cryo-EM images. The HPV 16 VLPs' diameter ranged from 35 nm to 50 nm.

FIG. 19 shows the stability of virus trapping by the shells. TEM quantification of AAV2 trapping in O shells subjected to a dilution series. The fraction of occupied shells remained the same prior and after 100-fold dilution and incubation for 14 days at RT in the diluted sample relative to the non-diluted sample. The overall shell concentration was 0.072 nM.

FIG. 20 shows TEM of rubella protein debris trapping in T1 shells. Successful encapsulation of protein debris from the rubella VLP sample.

FIG. 21 shows the results of negative stain TEM imaging of a virus cocktail trapping with heparan sulfate-modified T1 half-shells. (A,B) TEM fields of view of T1 half-shells trapping AAV2, Chikungunya and HPV16 virus particles in different ratios, with homogeneous and heterogeneous complexes. Selected trapped virus particles as (C) one Chikungunya, (D) one HPV16, (E) one AAV2, (F top) several AAV2, (F bottom) HPV16-Chik, (G) AAV2-Chik, (H) AAV2-HPV16. All scale bars: 100 nm.

FIG. 22 shows the cooperative effect of multiple O shells capturing numerous AAV2 particles. Scale bar: 50 nm.

### DETAILED DESCRIPTION OF THE INVENTION

The present disclosure provides constructs that enable the encapsulation of viruses or viral particles.

Unless defined otherwise, all technical and scientific terms used herein have the same meaning as commonly understood by those of ordinary skill in the art to which this invention pertains.

The terms “comprising” and “including” are used herein in their open-ended and non-limiting sense unless otherwise noted. With respect to such latter embodiments, the term “comprising” thus includes the narrower term “consisting of”.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, and the like, this is taken to mean also a single compound, salt, or the like.

Therefore, in one aspect, the present invention relates to a DNA-based nanostructure, wherein said DNA-based nanostructure is a shell comprising a cavity enclosed by said DNA-based nanostructure, wherein said DNA-based nanostructure is formed by self-assembling DNA-based building blocks, wherein each of said self-assembling DNA-based building blocks is formed by a single-stranded DNA template strand and a set of oligonucleotides complementary to said single-stranded DNA template, wherein each of said oligonucleotides is either complementary to one contiguous DNA sequence stretch or to at least two non-contiguous DNA sequence stretches on said single-stranded DNA template, wherein each of said self-assembling DNA-based building blocks is a triangular and/or a rectangular prismoid, particularly a triangular prismoid, and wherein a subset of one or more of said oligonucleotides in one or more of said self-assembling DNA-based building blocks is/are each linked to a construct comprising at least one sulfonated or sulfated polysaccharide group pointing to the interior of said cavity, particularly a construct comprising one or two sulfonated or sulfated polysaccharide groups, wherein each said construct comprises (i) a handle comprising at least one binding site for said sulfonated or sulfated polysaccharide group, and (ii) said sulfonated or sulfated polysaccharide group(s) bound to said handle.

In an alternative embodiment, a subset of one or more of said oligonucleotides in one or more of said self-assembling DNA-based building blocks is linked to a construct comprising at least one sialic acid group pointing to the interior of said cavity, particularly a construct comprising one or two sialic acid groups.

In particular embodiments, said handle has a length corresponding to at least the length of a single-stranded oligonucleotide comprising 30 nucleotides.

By using such extended versions of the linking moieties, it could surprisingly be shown that the number of DNA-based nanostructures that actually encapsulated a virus particle was drastically increased.

In the context of the present disclosure, the term “DNA-based nanostructure” refers to a nanostructure that is formed by a set of DNA-based macromolecules. DNA-based nanostructure of the type used in accordance with the present invention are described in detail in of WO 2021/165528 and in Sigl et al., loc. cit.

In the context of the present disclosure, the term “DNA” refers to deoxyribonucleic acid composed of a single-strand of monomeric units called nucleotides, wherein each nucleotide is composed of a nitrogen-containing nucleobase, a 2-deoxyribose sugar moiety, and a phosphate group, wherein the individual nucleotides are linked in the single-strand by a phosphate group linking the OH group in position 5′ of a 2-deoxyribose sugar moiety to the OH group in 3′ of a neighboring 2-deoxyribose sugar moiety. In particular embodiments, the nitrogen-containing nucleobases are independently selected from cytosine [C], guanine [G], adenine [A] and thymine [T]. In particular embodiments, one or more of the nucleobases are non-canonical bases, in particular a non-canonical base selected from the list of: a modified adenosine, in particular N6-carbamoyl-methyladenine or N6-methyadenine; a modified guanine, in particular 7-deazaguanine or 7-methylguanine; a modified cytosine, N4-methylcytosine, 5-carboxylcytosine, 5-formylcytosine, 5-glycosylhydroxymethylcytosine, 5-hydroxycytosine, or 5-methylcytosine; a modified thymidine, in particular α-glutamyl thymidine or α-putrescinyl thymine; a uracil or a modification thereof, in particular uracil, base J, 5-dihydroxypentauracil; or 5-hydroxymethyldeoxyuracil; deoxyarchaeosine and 2,6-diaminopurine. A stretch of a single strand of DNA may interact with a complementary stretch of DNA by interaction of complementary nucleobases, wherein cytosine and guanine, and adenine and thymine, are complementary to each other, respectively by forming two (A/T) and three (G/C) hydrogen bonds between the nucleobases. Two single-strands of DNA may be fully complementary to each other, as in the case of genomic DNA, or may be partially complementary to each other, including situations, where one single-strand of DNA is partially complementary to two or more other single-stranded DNA strands. The interaction of two complementary single-stranded DNA sequences results in the formation of a double-stranded DNA double helix.

As is well known, DNA has evolved in nature as carrier of the genetic information encoding proteins. DNA further includes non-coding regions that include regions having regulatory functions. Thus, any DNA-based application usually critically depends on the specific DNA sequence and is almost always only enabled by naming the specific DNA sequence. In contrast, in the context of the present invention, such coding and/or regulatory functions do not play any role and may or may not be present, since the underlying DNA sequences are solely designed and selected in a way that the desired arrangement of double-helical subunits is formed.

Thus, in one embodiment any form of a long single-stranded DNA sequence, whether naturally occurring DNA (such as the DNA of a bacteriophage) or synthetically produced DNA may be selected as template, and a set of short single-stranded DNA sequences may be designed, wherein each sequence is complementary to one or more different parts of the template and thus forms one or more double-helical sections. Collectively, all such double-helical sections created by interaction of the full set of short single-stranded DNA sequences with the template, then form the desired three-dimensional arrangement. Starting from a given single-stranded template sequence, the design of a set of complementary can be set up using known techniques, such as, for example, the methods described for the synthesis of megadalton-scale discrete objects with structurally well-defined 3D shapes (18, 24-35). In particular, iterative design with caDNAno (37) paired with elastic-network-guided molecular dynamics simulations (38) can be used.

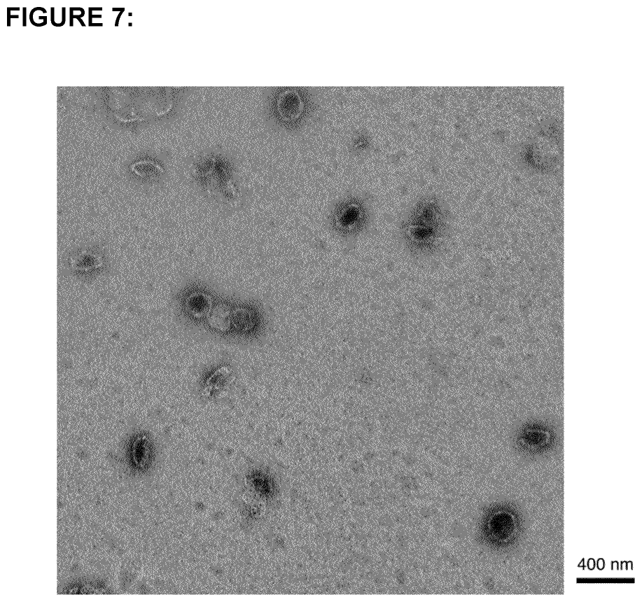

In addition to the interaction of complementary nucleobases of different stretches of single-stranded DNA via hydrogen bonds, additional interactions between different DNA strands are possible, including the stacking interactions between the blunt ends of two double-stranded DNA helices (36), thus enabling the design and the formation of complex DNA-based nanostructures via the shape-complementarity of double-helical subunits. Thus, two three-dimensional arrangements formed in accordance with the previous paragraph, may interact with each other by stacking interactions between double-helical subunits present on the two three-dimensional arrangements, including specific interactions between two three-dimensional arrangements having complementary protrusions and recessions (or knobs and holes), as shown, for example, in FIGS. 7-13D of WO 2021/165528.

In an alternative aspect of the present invention, the invention relates to a macromolecule-based nanostructure, which is an RNA-based nanostructure.

In the context of the present disclosure, the term “RNA” refers to ribonucleic acid composed of a single-strand of monomeric units called nucleotides, wherein each nucleotide is composed of a nitrogen-containing nucleobase, a ribose sugar moiety, and a phosphate group, wherein the individual nucleotides are linked in the single-strand by a phosphate group linking the OH group in position 5′ of a ribose sugar moiety to the OH group in 3′ of a neighboring ribose sugar moiety. In particular embodiments, the nitrogen-containing nucleobases are independently selected from cytosine [C], guanine [G], adenine [A] and uracil [U]. In particular embodiments, one or more of the nucleobases are non-canonical bases, in particular a non-canonical base selected from the list of: pseudouridine, ribothymidine, and inosine. Unlike DNA, RNA is most often in a single-stranded form, but the formation of double-stranded forms is possible by interaction of complementary nucleobases, wherein cytosine and guanine, and adenine and uracil, are complementary to each other, respectively by forming two (A/U) and three (G/C) hydrogen bonds between the nucleobases.

In the context of the present invention, the term “cavity” relates to the space enclosed by said DNA-based nanostructure. In particular embodiments, said cavity resembles a sphere, where a spherical segment has been cut off, with the cutting plane being formed by the self-assembling DNA-based building blocks at the borders of said DNA-based nanostructure. In particular embodiments, the cutting plane is a great circle so that the DNA-based nanostructure is a half-shell. In other cases, where the three-dimensional geometric shape of said DNA-based nanostructure is derived from a spherocylinder or a polyhedron, in particular a tetrahedron, an octahedron or an icosahedron, said cavity is to be understood as the space resulting from cutting a corresponding spherocylinder or polyhedron by a plane with the cutting plane being formed by the self-assembling DNA-based building blocks at the borders of said DNA-based nanostructure.

In certain other embodiments, said DNA-based nanostructure resembles a spherical segment. Since in such embodiments, only part of a virus interacting with such DNA-based nanostructure is covered, the encapsulation of one or more viruses or viral particles in accordance with the present invention requires binding of two or more of such DNA-based nanostructures to said one or more viruses or viral particles.

In the context of the present application, the term “sulfonated or sulfated polysaccharide group” relates to a group comprising a polysaccharide comprising at least one sulfated hydroxy group or at least one sulfonated glycosylamino group.

Importantly, in addition to targeting specific receptors, many viruses also weakly interact with different biological substances, including sulfated of sulfonated polysaccharides (Cagno, V. et al., Viruses 11 (2019) 596; see Table 2).

In particular embodiments, each of said handles comprises two binding sites for said sulfonated or sulfated polysaccharide groups.

In particular embodiments, each of said polysaccharide comprising at least one sulfated hydroxy group or at least one sulfonated glycosylamino group is independently selected from the list of heparin, heparan sulfate, hybrid heparan sulfates, carrageenans, cellulose sulfate, and dextrin 2-sulfate, in particular a heparan sulfate or a hybrid heparan sulfate.

In particular embodiments, said polysaccharide comprising at least one sulfated hydroxy group or at least one sulfonated glycosylamino group consists of between 3 and 10 disaccharide units, in particular 4, 5, 6, 7, 8 of 9 units, particularly 4 or 9 monosaccharide units.

In particular embodiments, said disaccharide units comprise two or three O- and/or N-sulfonate groups per disaccharide unit, in particular three O- and/or N-sulfonate groups.

In particular embodiments, each of said sulfonated or sulfated polysaccharide groups is independently selected from the list of heparin, heparan sulfate, hybrid heparan sulfates, carrageenans, cellulose sulfate, and dextrin 2-sulfate.

In particular such embodiments, each of said sulfonated or sulfated polysaccharides is independently selected from a heparan sulfate and a hybrid heparan sulfate, in particular is a heparan sulfate.

In the context of the present invention, the terms “heparin” and “heparan sulfate” both relate to a family of linear sulfated, heterogeneous polysaccharides found on the cell membrane and in the extracellular matrix as part of heparan sulfate proteoglycans (HSPGs). They are composed of repeating 1→4 linked disaccharide units, in which one monosaccharide is an α-D-glucosamine residue and the other an uronic acid (or, in a salt form, an uronate). Heparin is a structurally similar polysaccharide found within mast cells as a component of serglycin proteoglycans. Heparan sulfate and heparin can be defined as follows: first, in heparin, the uronates are predominantly α-L-iduronate, whereas in heparan sulfate, the uronates are mainly, β-D-glucuronates, the C-5 epimers of α-L-iduronate. Second, in heparan sulfate, the D-glucosamine residues are predominantly N-acetylated, whereas in heparin, they are N-sulfonated. Finally, whereas at least 70-80% of heparin is composed of the disaccharide L-iduronate 2-O-sulfate a (1→4) D-glucosamine N,6-sulfate, in heparan sulfate around 40-60% of the disaccharides consist of (1→4) D-glucuronate β (1→4) D-glucosamine, that can be either N-acetylated or N-sulfonated. Together, these structural characteristics make heparin more sulfated and, hence, more charged than heparan sulfate. It has become apparent, however, that the designations heparin or heparan sulfate are less clear-cut than this description implies, and that polysaccharides isolated from some organisms appear to be hybrid constructs. In the context of the present invention, the term “hybrid heparan sulfate” is used to refer to such hybrids having structures being a mixture of the “typical” heparin structural elements (L-iduronates; high degree of sulfonation) and the “typical” heparan sulfate structural elements (D-glucuronate; N-acetylation and 6-O-sulfonation).

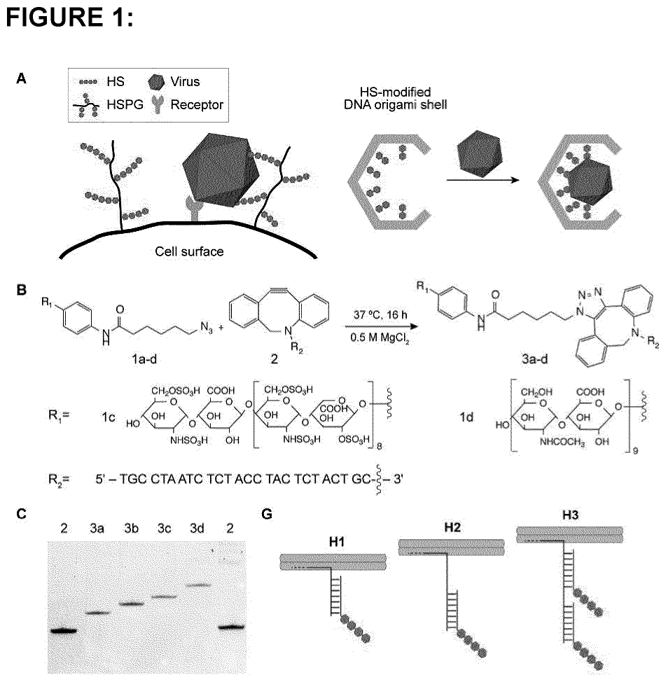

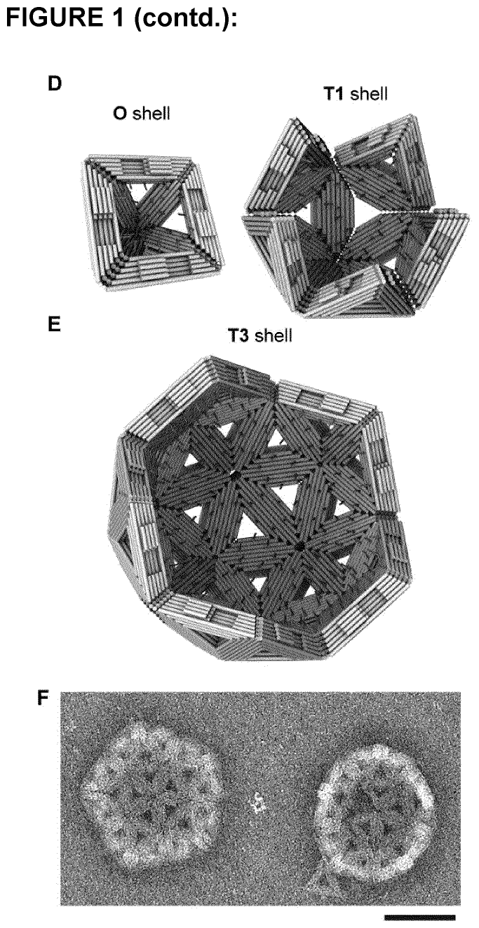

Heparan sulfate proteoglycans (HSPG) (Cagno, V. et al., Viruses 11 (2019) 596; Zhang, Q. et al., Cell Discov. 6 (2020) 1-14) are commonly found on the surface of mammalian cells. The weak interactions of viruses with HSPG are conserved across virus families and thus appear generically beneficial for the virus lifecycle. For example, HSPG-virus interactions may enable an infection-enhancing diffusive search of virus particles for their specific host cell receptors on the surface of cells (FIG. 1A, left panel). The interactions of heparan sulfate (HS) with viruses have already been exploited for medical purposes, for example in virus-sequestering coatings of condoms that are based on HS-decorated dendrimers (Tyssen, D. et al., PLOS ONE 5, e12309 (2010); Price, C. F. et al., PLOS ONE 6, e24095 (2011); Zelikin, A. N. & Stellacci, F., Adv. Healthc. Mater. 10 (2021) 2001433). Other investigations have frequently involved the surface functionalization of nanoparticles and polymers with HS derivatives to create virus-binding complexes with antiviral activity (Cagno, V. et al., Nat. Mater. 17 (2018) 195-203; Al-Mahtab, M. et al., PLOS ONE 11 (2016) e0156667; Vaillant, A., Antiviral Res. 133 (2016) 32-40; Cagno, V. et al., Antimicrob. Agents Chemother. 64 (2020) e02001-20). Commonly, a high level of multivalency is required to increase the strength of binding between the HS-nanoparticles and viruses. The reversible nature of the binding can lead to undesirable unbinding and release of infectious viruses from the virus-sequestering coatings, or the requirement for high concentrations of the therapeutically active agent to be maintained (Zelikin, loc. cit.).

In particular embodiments, said subset of self-assembling DNA-based building blocks consists of between 1 and 100% of all self-assembling DNA-based building blocks forming said DNA-based nanostructure. In particular embodiments, said subset of self-assembling DNA-based building blocks consists of between 50 and 100% of all self-assembling DNA-based building blocks forming said DNA-based nanostructure, more particularly between 75 and 100%, and in particular 100% of all self-assembling DNA-based building blocks.

In particular embodiments, one or more of said self-assembling DNA-based building blocks in said subset comprise n single-stranded oligonucleotides as said handles, wherein each handle is independently linked to at least one of said sulfonated or sulfated polysaccharide groups, wherein n is an integer independently selected from 1, 2, 3, 4, 5, 6, 7, 8, 9, 10, 11, and 12, particularly wherein n is 9.

In a particular embodiment, said handles are single-stranded oligonucleotides having a length of between 30 and 60 nucleotides, in particular between 40 and 55 nucleotides, more particularly between 45 and 50 nucleotides.

Examples of self-assembling DNA-building blocks in the form of frusta, wherein the small base of each of said frusta comprises nine of said polynucleotides are shown in the examples, for example T_octa self-assembling DNA-based building blocks, T1_pentamer_triangle self-assembling DNA-based building blocks, T1_ring_triangle self-assembling DNA-based building blocks or T3_6_triangle based self-assembling DNA-based building blocks.

In particular embodiments, each member of said n oligonucleotides comprises at least one oligonucleotide stretch as binding site, and wherein each of said sulfonated or sulfated polysaccharide groups comprises an oligonucleotide having a sequence that is complementary to one of said oligonucleotide stretches comprised in said handles. In particular embodiments, each member of said n oligonucleotides comprises one or two oligonucleotide stretches as binding sites.

In particular embodiments, each member of said n oligonucleotides comprises two oligonucleotide stretches as binding sites.

By using oligonucleotides comprising two binding sites, it could surprisingly be shown that the number of DNA-based nanostructures that actually encapsulated a virus particle was further increased, even when compared to the already advantageous version with an extended handle design described above. This is particularly surprising in light of the fact that the additional second binding site is at the same position as the initial binding site in the H1 handle design with a short oligonucleotide (26-mer) and despite the fact that the addition of a second binding site and HS moiety might have been expected to decrease the encapsulation efficiency due to steric hindrance.

Examples of subsets of nine of such oligonucleotide handles, which can be linked to constructs comprising a sulfonated or sulfated polysaccharide group, can be found in the sets of Hx oligos according to SEQ ID. NOs: 177-185, 186-194, 389-397, 398-406, 607-615, 616-624, 821-829, 1018-1026, 1216-1224, 1416-1424, 1613-1621, and 1812-1820, each oligo comprising one handle, and the sets of Hx oligos according to SEQ ID. NOs: 195-203, 407-415, and 625-633, each oligo comprising two binding sites.

In a particular embodiment, said cavity has a diameter of at least 15 m, at least 25 nm, at least 50 nm, at least 100 nm, at least 150 nm, at least 200 nm or at least 250 nm.

In particular embodiments, said cavity has a diameter of at most 1,000 nm.

In the context of the present invention, the term “diameter” refers to the diameter of the smallest circle that is encompassed by the surface of the DNA-based nanostructure. For the sake of clarity, in the case of a DNA-based nanostructure in the form of a capsule (or spherocylinder), the diameter is the diameter of the hemispherical ends and/or the diameter of the cylindrical central part.

In a particular embodiment, the DNA-based nanostructure has a molecular mass of at least 1 MDa, particularly at least 10 MDa, particularly at least 20 MDa, more particularly at least 30 MDa. In other particular embodiments, the DNA-based nanostructure has a molecular mass of at least 50 MDa, at least 80 MDa, at least 100 MDa, at least 200 MDa, or at least 500 MDa. In particular embodiments, the DNA-based nanostructure has a molecular mass of at most 1,500 MDa.

In particular embodiments, the ratio between the numerical value of the molecular mass of the DNA-based nanostructure (in MDa) and the numerical value of the volume of the cavity encased by said DNA-based nanostructure (in nm³) is less than 10,000, particularly less than 9,000. In particular embodiments said ratio has a value of between 1,000 and 10,000, particularly between 2,000 and 9,000. For example, in the case of certain octahedral nanostructures, where the molecular mass is about 40 MDa, and where the encased volume is about 113,000 nm³, said ratio is about 2,800.

In particular embodiments, the ratio between the outer surface area of the DNA-based nanostructure covered by the macromolecules forming said DNA-based nanostructure and the outer surface area not covered by said macromolecules (excluding the area of the opening of a DNA-based nanostructure in the form of a shell) is at least 1, in particular at least 2, in particular at least 4, in particular at least 6, in particular at least 8. In other particular embodiments, the ratio is at least 10. In particular embodiments, the ratio is between 1 and 20, in particular between 2 and 18, between 4 and 16, between 6 and 14, and more particularly between 8 and 12. For example, in a case, where the DNA-based nanostructure is a shell in the form of a half sphere, only the area of the curved surface, but not that of the opening, i.e. the area of the flat face of the half sphere, is used for calculating said ratio.

In a particular embodiment, the molecular weight of each self-assembling DNA-based building block is between 4.5 and 5.5 MDa.

In a particular embodiment, each self-assembling DNA-based building block comprises between 7,500 and 8,500 base pairs.

In a particular embodiment, the DNA-based nanostructure consists of between 4 and 180 of such self-assembling DNA-based building blocks.

In particular embodiments, said single-stranded DNA template is single-stranded DNA of filamentous bacteriophage, or is derived from single-stranded DNA of filamentous bacteriophage.

In the context of the present invention, the term “filamentous bacteriophage” refers to a type of bacteriophage, or virus of bacteria, which is characterized by its filament-like shape that usually contains a genome of circular single-stranded DNA and infects Gram-negative bacteria. Filamentous phage includes Ff phage, such as M13, f1 and fd1 phage, and Pf1 phage.

In particular embodiments, said single-stranded DNA template has the sequence of SEQ ID NO: 1 (M13 8064) (see Table 1). In particular embodiments, said single-stranded DNA is circular.

In the context of the present invention, a single-stranded DNA template that is “derived from single-stranded DNA of filamentous bacteriophage” refers to a DNA construct that is derived from a naturally occurring of published DNA sequence of a filamentous bacteriophage by one or more of: (i) opening of the circular structure to a linear sequence; (ii) deletion of one or more nucleotides; (iii) insertion of one or more nucleotides; (iii) substitution of one or more nucleotides; (iv) addition of one or more nucleotides; and (v) modification of one or more nucleotides. While any such variation might have detrimental, or at least rather unpredictable, effects on bacteriophage biology, its infectivity and its ability to propagate, such effects do not play any role in the context of the present invention, since, as already mentioned above, said single-stranded DNA template is only used as naked template without any requirement for having any functional property, and all structural aspects, such as the correct formation the three-dimensional shape of said self-assembling DNA-based building blocks, are implemented by the proper choice of said set of complementary oligonucleotides.

In particular embodiments, said single-stranded DNA template has at least 80%, particularly at least 90%, more particularly at least 95%, sequence identity to the sequence of a naturally occurring or published sequence of a filamentous bacterio-phage, in particular to a M13, f1 or fd1 phage, in particular to a sequence selected from SEQ ID NO: 1 (M13 8064) and M13 7249 (SEQ ID NO: 2 of WO 2021/165528).

In a particular embodiment, the DNA-based nanostructure is a closed three-dimensional geometric shape, in particular a closed three-dimensional geometric shape selected from a sphere, a spherocylinder, and a polyhedron, in particular a tetrahedron, an octahedron or an icosahedron, formed in situ from said self-assembling DNA-based building blocks in the presence of said viruses or viral particles to be encapsulated.

In a particular such embodiments, said self-assembling DNA-based building blocks forming said DNA-based nanostructure in situ are a collection of individual DNA-based building blocks, each comprising a single single-stranded DNA template.

In particular other embodiments, said self-assembling DNA-based building blocks forming said DNA-based nanostructure in situ are a collection of one or more preassembled DNA-based building blocks consisting of two or more individual DNA-based building blocks, each comprising a single single-stranded DNA template.

In certain embodiments, all of said self-assembling DNA-based building blocks are preassembled DNA-based building blocks. In an alternative embodiment, said self-assembling DNA-based building blocks are a mixture of preassembled DNA-based building blocks and of individual DNA-based building blocks, each comprising a single single-stranded DNA template.

In such embodiments, said preassembled DNA-based building blocks form a curved geometrical shape, wherein said handles or said constructs comprising at least one sulfonated or sulfated polysaccharide group, which are linked to said handles, are present on the negative curvature of said curved geometrical shape, so that said handles or constructs are pointing to the interior of the cavity formed from the self-assembly of said preassembled DNA-based building blocks.

In another particular embodiment, the DNA-based nanostructure is a shell with an opening for accessing said cavity.

In the context of the present invention, the term “shell” refers to a structure that is a part of a closed three-dimensional geometric shape, in particular a closed three-dimensional geometric shape selected from a sphere, a spherocylinder, and a polyhedron, in particular a tetrahedron or an octahedron,

In yet another particular embodiment, the DNA-based nanostructure is a combination of a first subshell and a second subshell, each with an opening to access a first and a second inner cavity, respectively, wherein said first and said second inner cavity together form said cavity.

In a particular embodiment, said first and said second subshells are connected by at least one linker.

In particular embodiments, said linker is a linker selected from a DNA linker, an RNA linker, a polypeptide linker, a protein linker and a chemical linker.

In the context of the present invention, the term “DNA linker” refers to a linker formed from DNA, wherein the sequence of said DNA linker is not complementary to the DNA of said single-stranded DNA template or to any of said set of oligonucleotides complementary to said single-stranded DNA template, wherein said DNA linker is linked at one terminus to a DNA sequence forming a self-assembling DNA-based building block of said first shell, and at the other terminus to a DNA sequence forming a self-assembling DNA-based building block of said second shell.

In the context of the present invention, the term “polypeptide linker” refers to a linker formed from at least 2, particularly at least 5, at least 10, or at least 20 amino acid residues linked by peptide bonds, wherein said polypeptide has no tertiary or quaternary structure, and wherein said polypeptide linker is linked at one terminus to a DNA sequence forming a self-assembling DNA-based building block of said first shell, and at the other terminus to a DNA sequence forming a self-assembling DNA-based building block of said second shell.

In the context of the present invention, the term “protein linker” refers to a linker formed from at least 20, particularly at least 50, at least 100, at least 200 amino acid residues, at least 500 amino acid residues, or at least 1,000 amino acid residues, particularly less than 1,500 amino acid residues linked by peptide bonds, wherein said polypeptide has tertiary and/or quaternary structure, and wherein said protein linker is linked at one terminus to a DNA sequence forming a self-assembling DNA-based building block of said first shell, and at the other terminus to a DNA sequence forming a self-assembling DNA-based building block of said second shell. In particular embodiments, said protein linker is covalently attached to said DNA sequences. In particular other embodiments, said protein linker is non-covalently attached to said DNA sequences, in particular, wherein said protein linker is an antibody-based protein linker, in particular selected from a diabody and a full antibody, including an IgG antibody.

In the context of the present invention, the term “chemical linker” refers to a continuous chain of between 1 and 30 atoms (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 atoms; thus, in the context of the present invention, the term “between” is used so that the borders mentioned are included) in its backbone, i.e. the length of the linker is defined as the shortest connection as measured by the number of atoms or bonds between the two DNA sequences linked by said chemical linker. In the context of the present invention, a chemical linker preferably is an C₁₋₂₀-alkylene, C₁₋₂₀-heteroalkylene, C₂₋₂₀-alkenylene, C₂₋₂₀-heteroalkenylene, C₂₋₂₀-alkynylene, C₂₋₂₀-heteroalkynylene, cycloalkylene, heterocycloalkylene, arylene, heteroarylene, aralkylene, or a heteroaralkylene group, which may optionally be substituted. The linker may contain one or more structural elements such as carboxamide, ester, ether, thioether, disulfide, urea, thiourea, hydrocarbon moieties and the like. The linker may also contain combinations of two or more of these structural elements. Each one of these structural elements may be present in the linker more than once, e.g. twice, three times, four times, five times, or six times. In some embodiments the linker may comprise a disulfide bond. It is understood that the linker has to be attached either in a single step or in two or more subsequent steps to the two DNA sequences linked by said chemical linker. To that end the linker to be will carry two groups, preferably at a proximal and distal end, which can (i) form a covalent bond to a group present in one of the two DNA sequences to be linked, or (ii) which is or can be activated to form a covalent bond with one of the two DNA sequences.

In a particular embodiment, the DNA-based nanostructure is based on an icosahedral structure.

In a particular embodiment, each of said self-assembling DNA-based building blocks is a prismoid.

In the context of the present invention, the term “prismoid” refers to a polyhedron, wherein all vertices lie in two parallel planes.

In particular embodiments, said prismoid is a triangular prismoid. In other embodiments, said prismoid is a rectangular prismoid.

In particular embodiments, the DNA-based nanostructure is based on a mixture of a triangular and a rectangular prismoid.

In a particular embodiment, the present invention relates to a DNA-based nanostructure, wherein each said triangular, or said rectangular prismoid, is formed by m triangular, or rectangular, respectively, planes, wherein m is an integer independently selected from 4, 5, 6, 7 and 8, in particular independently selected from 5, 6 and 7, more particularly wherein said integer is 6.
wherein the three, or four, respectively, edges of each of said m planes are formed by n parallel stretches of DNA double helices, wherein n is an integer independently selected from 1, 2, 3, 4, 5 and 6 in particular independently selected from 2, 3, 4 and 5, more particularly independently selected from 3 and 4,
wherein each plane is connected to a plane above and/or a plane beyond said plane (i) by stacking interactions between the DNA double helices forming said planes, and (ii) partially by DNA stretches within said single-stranded DNA template and/or said oligonucleotides forming said DNA-based building block bridging at least two of said planes, and
wherein at least two of the three, or four, respectively, side trapezoids comprise a specific pattern of recesses and/or extrusions formed by missing or additional DNA double helical stretches for specific interaction with a complementary pattern on the side trapezoid of another one of said self-assembling DNA-based building blocks.

In a particular embodiment, the average length of each of the n stretches of DNA double helices in the m planes of a triangular, or rectangular, respectively, prismoid is between 80 and 200 base pairs.

In particular embodiments, said triangular prismoid is a triangular frustum. In particular embodiments, said rectangular prismoid is a rectangular frustum.

In the context of the present invention, the term “triangular frustum” refers to a three-dimensional geometric shape in the form of a triangular pyramid, and the term “rectangular frustum” refers to a three-dimensional geometric shape in the form of a rectangular pyramid, where the tip of the pyramid has been removed resulting in a plane on the top parallel to the basis of the pyramid.

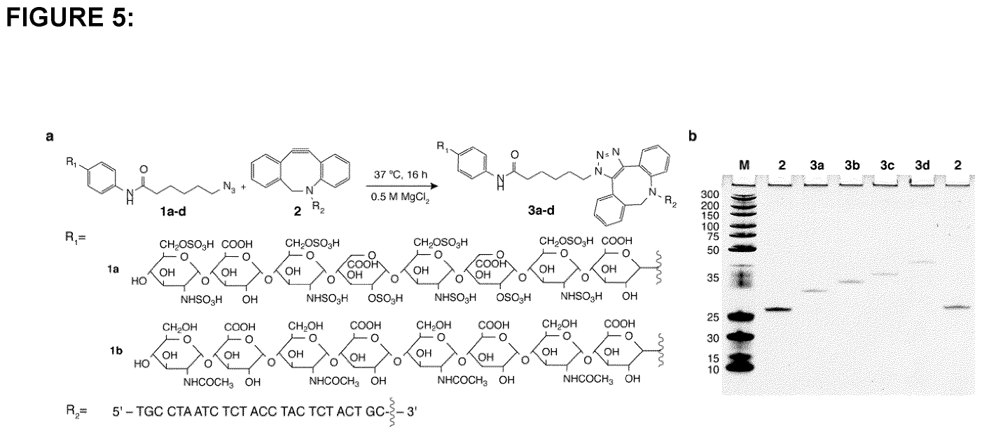

In a particular embodiment, for at least part of said self-assembling DNA-based building blocks the length of at least one edge of each of said m planes is decreasing from the first to the m^{th} plane, so that a bevel angle results between planes perpendicular to said first plane and the trapezoid plane formed by said m edges (see FIG. 5 of WO 2021/165528). In particular embodiments, all three, or four, respectively, trapezoid planes exhibit a bevel angle.

In a particular embodiment, a bevel angle is between 16° and 26°, particularly between 18° and 24°, more particularly between 20° and 22°, most particularly about 20.9°.

In a particular embodiment, said DNA-based nanostructure comprises at least one set of self-assembling DNA-based building blocks, wherein all three, or four, respectively, side trapezoids comprise a specific pattern of recesses and/or extrusions formed by missing or additional DNA double helical stretches for specific interaction with a complementary pattern on the side trapezoid of another one of said self-assembling DNA-based building blocks.

In a particular embodiment, particularly in the case of a DNA-based nanostructure closed three-dimensional geometric shape, all said self-assembling DNA-based building blocks are identical.

In a particular embodiment, said DNA-based nanostructure comprises two or more sets of self-assembling DNA-based building blocks.

In a particular embodiment, said DNA-based nanostructure is rod-shaped.

In particular embodiments, said DNA-based nanostructure comprises two or more sets of self-assembling DNA-based building blocks.

In particular such embodiment, said rod-shaped DNA-based nanostructure comprises at least a first and a second set of self-assembling DNA-based building blocks, wherein said first and set second set differ at least with respect to the bevel angles. In a particular embodiment, at least one set consists of self-assembling DNA-based building blocks exhibiting only two bevel angles. In a particular embodiment, said at least one set consists of rectangular frusta, which comprise a bevel angle on each of two opposing trapezoids.

In a particular embodiment, the side trapezoids forming the rim of said shell, or of said first and second shell, respectively, do not comprise a specific pattern of recesses and/or extrusions formed by missing or additional DNA double helical stretches for specific interaction with a complementary pattern on the side trapezoid of another one of said self-assembling DNA-based building blocks.

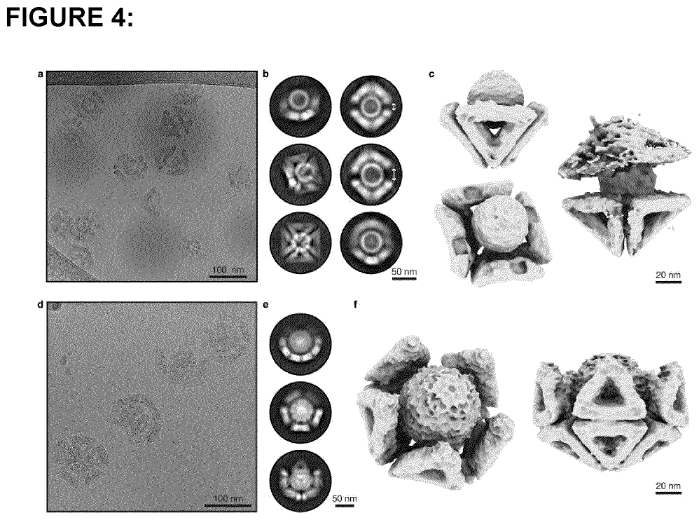

In a particular embodiment, said DNA-based nanostructure is a shell selected from
(i) a half octahedron T_octa (FIG. 4A of WO 2021/165528), which consists of a set of four copies of a triangular frustum, wherein the base-pair stacking contacts on one of the triangular edges of the triangular frustum are inactivated by either strand shortening or by adding unpaired thymidines (FIG. 4A of WO 2021/165528, see FIG. 24A,D of WO 2021/165528);
(ii) a half T=1 shell (FIG. 4B of WO 2021/165528), which consists of two sets of in each case five copies of two different triangular frusta, wherein the five copies of the first set form a closed pentamer, and the five copies of the second set dock onto the edges of said pentamer (FIG. 4B of WO 2021/165528, see FIG. 24B,E of WO 2021/165528); and
(iii) a “trap” T=1 shell with a missing pentagon vertex (FIG. 4C of WO 2021/165528), which consists of three sets of in each case five copies of three different triangular frusta, wherein the five copies of the first set form a closed pentamer, the five copies of the second set dock onto the edges of said pentamer the five copies of the second set dock onto the edges of said pentamer, and the five copies of the third set dock into the gaps between the five copies of said second set (FIG. 4C of WO 2021/165528, see FIG. 24C,F of WO 2021/165528);
(iv) a T=3 icosahedral half shell, which consists of a total of 30 triangular subunits partitioned as five copies of six different full-size DNA triangle designs with specific edge docking rules

In a particular embodiment, the present invention relates to a DNA-based nanostructure further comprising one or more types of DNA brick constructs, each type of such DNA brick constructs being characterized by one or more interaction sites for specific interaction by edge-to-edge stacking contacts with one or more complementary interaction sites present on the plane of said triangular, or rectangular, respectively, prismoid on the outer surface of said DNA-based nanostructure, wherein said DNA brick constructs cover the free space between the three, or four, respectively, edges of said plane (see FIG. 33 of WO 2021/165528).

In a particular embodiment, the present invention relates to a DNA-based nanostructure further comprising one or more cross-linkages within one of said triangular, or rectangular, respectively, prismoids, and/or between two of said triangular, or rectangular, respectively, prismoids.

In the context of the present invention, the term “cross-linkage” refers to any permanent or intermittent linkage within one of said triangular, or rectangular, respectively, prismoids, and/or between two of said triangular, or rectangular, respectively, prismoids. Any such linkage may be achieved a priori by linking two of the oligonucleotides being used for forming the self-assembling DNA-based building blocks prior to the assembly, or a priori, e.g. by chemically or photochemically adding linkages between different parts of the three-dimensional nanostructure. Permanent linkages may, for example, be created by photochemically cross-linking T residues appropriately positioned in the structure under formation of covalent cyclobutane pyrimidine dimer (CPD) bonds (41), and intermittent linkages may, for example, be created by photochemically cross-linking the blunt ends of two double-helical subunits between a 3-cyanovinylcarbazole (cnvK) moiety positioned at a first blunt end and a thymine residue (T) positioned at the other blunt end (40).

In a second aspect, the present invention relates to a composition comprising a DNA-based nanostructure according to the present invention encapsulating one or more viruses or viral particles.

In particular embodiments, said composition is formed in a process of removing said viruses or viral particles from a medium containing said viruses or viral particles. In particular other embodiments, said composition is formed in a process of incorporating said one or more viruses or viral particles as cargo in said DNA-based nanostructure.

In a third aspect, the present invention relates to a method for encapsulating a one or more viruses or viral particles, comprising the steps of: providing a DNA-based nanostructure according to the present invention, and contacting said DNA-based nanostructure with a medium comprising, or suspected to comprise, said viruses or viral particles.

In particular embodiments, (i) a DNA-based half shell nanostructure based on T_octa self-assembling DNA-based building blocks is selected for a virus of a size up to 50*50*50 nm³; (ii) a DNA-based half shell nanostructure based on T1_pentamer_triangle self-assembling DNA-based building blocks is selected for a virus of a size between 15*15*15 and 100*100*100 nm³; (iii) a DNA-based half shell nanostructure based on a combination of T1_pentamer_triangle and T1_ring_triangle self-assembling DNA-based building blocks is selected for a virus of a size between 15*15*15 and 100*100*100 nm³; and/or (iv) a DNA-based half shell nanostructure based on T3_6_triangle self-assembling DNA-based building blocks is selected for a virus of a size of 50*50*50 nm³ or larger.

In particular embodiments, said method is for removing said one or more viruses or viral particles from said medium. In particular embodiment, said method is for encapsulating said one or more viruses or viral particles in order to transport said virus or viral particle.

In a fourth aspect, the present invention relates to a method for encapsulating one or more viruses or viral particles, comprising the steps of: providing a DNA-based nanostructure according to the present invention, and contacting said DNA-based nanostructure with a medium comprising, or suspected to comprise, said viruses or viral particles.

In an alternative aspect, the disclosure provides a method for encapsulating one or more viruses or viral particles, comprising the steps of: adding self-assembling DNA-based building blocks to a medium comprising, or suspected to comprise, said viruses or viral particles resulting in the in situ formation of DNA-based nanostructure according to the present invention encapsulating one or more of said viruses or viral particles.

In yet another aspect, the disclosure provides a method for encapsulating a cargo different from a virus or viral particle, such as a complex macromolecule, comprising the steps of: providing a DNA-based nanostructure according to the present invention, and contacting said DNA-based nanostructure with a medium comprising, or suspected to comprise, said cargo.

In an alternative aspect, the disclosure provides a method for encapsulating cargo different from a virus or viral particle, such as a complex macromolecule, comprising the steps of: adding self-assembling DNA-based building blocks to a medium comprising, or suspected to comprise, said cargo different from a virus or viral particle, such as a complex macromolecule, resulting in the in situ formation of DNA-based nanostructure according to the present invention encapsulating said cargo different from a virus or viral particle, such as a complex macromolecule . . .

TABLE 1
M13 8064 Template Sequence

| SEQ ID | |
|---|---|
| NO: | Sequence |
| 1 | GGCAATGACCTGATAGCCTTTGTAGATCTCTCAAAAATAGCTACCCTCTCCGGCATTAATTTATCAGCTAGAA |
| | CGGTTGAATATCATATTGATGGTGATTTGACTGTCTCCGGCCTTTCTCACCCTTTTGAATCTTTACCTACACA |
| | TTACTCAGGCATTGCATTTAAAATATATGAGGGTTCTAAAAATTTTTATCCTTGCGTTGAAATAAAGGCTTCT |
| | CCCGCAAAAGTATTACAGGGTCATAATGTTTTTGGTACAACCGATTTAGCTTTATGCTCTGAGGCTTTATTGC |
| | TTAATTTTGCTAATTCTTTGCCTTGCCTGTATGATTTATTGGATGTTAATGCTACTACTATTAGTAGAATTGA |
| | TGCCACCTTTTCAGCTCGCGCCCCAAATGAAAATATAGCTAAACAGGTTATTGACCATTTGCGAAATGTATCT |
| | AATGGTCAAACTAAATCTACTCGTTCGCAGAATTGGGAATCAACTGTTATATGGAATGAAACTTCCAGACACC |
| | GTACTTTAGTTGCATATTTAAAACATGTTGAGCTACAGCATTATATTCAGCAATTAAGCTCTAAGCCATCCGC |
| | AAAAATGACCTCTTATCAAAAGGAGCAATTAAAGGTACTCTCTAATCCTGACCTGTTGGAGTTTGCTTCCGGT |
| | CTGGTTCGCTTTGAAGCTCGAATTAAAACGCGATATTTGAAGTCTTTCGGGCTTCCTCTTAATCTTTTTGATG |
| | CAATCCGCTTTGCTTCTGACTATAATAGTCAGGGTAAAGACCTGATTTTTGATTTATGGTCATTCTCGTTTTC |
| | TGAACTGTTTAAAGCATTTGAGGGGGATTCAATGAATATTTATGACGATTCCGCAGTATTGGACGCTATCCAG |
| | TCTAAACATTTTACTATTACCCCCTCTGGCAAAACTTCTTTTGCAAAAGCCTCTCGCTATTTTGGTTTTTATC |
| | GTCGTCTGGTAAACGAGGGTTATGATAGTGTTGCTCTTACTATGCCTCGTAATTCCTTTTGGCGTTATGTATC |
| | TGCATTAGTTGAATGTGGTATTCCTAAATCTCAACTGATGAATCTTTCTACCTGTAATAATGTTGTTCCGTTA |
| | GTTCGTTTTATTAACGTAGATTTTTCTTCCCAACGTCCTGACTGGTATAATGAGCCAGTTCTTAAAATCGCAT |
| | AAGGTAATTCACAATGATTAAAGTTGAAATTAAACCATCTCAAGCCCAATTTACTACTCGTTCTGGTGTTTCT |
| | CGTCAGGGCAAGCCTTATTCACTGAATGAGCAGCTTTGTTACGTTGATTTGGGTAATGAATATCCGGTTCTTG |
| | TCAAGATTACTCTTGATGAAGGTCAGCCAGCCTATGCGCCTGGTCTGTACACCGTTCATCTGTCCTCTTTCAA |
| | AGTTGGTCAGTTCGGTTCCCTTATGATTGACCGTCTGCGCCTCGTTCCGGCTAAGTAACATGGAGCAGGTCGC |
| | GGATTTCGACACAATTTATCAGGCGATGATACAAATCTCCGTTGTACTTTGTTTCGCGCTTGGTATAATCGCT |
| | GGGGGTCAAAGATGAGTGTTTTAGTGTATTCTTTTGCCTCTTTCGTTTTAGGTTGGTGCCTTCGTAGTGGCAT |
| | TACGTATTTTACCCGTTTAATGGAAACTTCCTCATGAAAAAGTCTTTAGTCCTCAAAGCCTCTGTAGCCGTTG |
| | CTACCCTCGTTCCGATGCTGTCTTTCGCTGCTGAGGGTGACGATCCCGCAAAAGCGGCCTTTAACTCCCTGCA |
| | AGCCTCAGCGACCGAATATATCGGTTATGCGTGGGCGATGGTTGTTGTCATTGTCGGCGCAACTATCGGTATC |
| | AAGCTGTTTAAGAAATTCACCTCGAAAGCAAGCTGATAAACCGATACAATTAAAGGCTCCTTTTGGAGCCTTT |
| | TTTTTGGAGATTTTCAACGTGAAAAAATTATTATTCGCAATTCCTTTAGTTGTTCCTTTCTATTCTCACTCCG |
| | CTGAAACTGTTGAAAGTTGTTTAGCAAAATCCCATACAGAAAATTCATTTACTAACGTCTGGAAAGACGACAA |
| | AACTTTAGATCGTTACGCTAACTATGAGGGCTGTCTGTGGAATGCTACAGGCGTTGTAGTTTGTACTGGTGAC |
| | GAAACTCAGTGTTACGGTACATGGGTTCCTATTGGGCTTGCTATCCCTGAAAATGAGGGTGGTGGCTCTGAGG |
| | GTGGCGGTTCTGAGGGTGGCGGTTCTGAGGGTGGCGGTACTAAACCTCCTGAGTACGGTGATACACCTATTCC |
| | GGGCTATACTTATATCAACCCTCTCGACGGCACTTATCCGCCTGGTACTGAGCAAAACCCCGCTAATCCTAAT |
| | CCTTCTCTTGAGGAGTCTCAGCCTCTTAATACTTTCATGTTTCAGAATAATAGGTTCCGAAATAGGCAGGGGG |
| | CATTAACTGTTTATACGGGCACTGTTACTCAAGGCACTGACCCCGTTAAAACTTATTACCAGTACACTCCTGT |
| | ATCATCAAAAGCCATGTATGACGCTTACTGGAACGGTAAATTCAGAGACTGCGCTTTCCATTCTGGCTTTAAT |
| | GAGGATTTATTTGTTTGTGAATATCAAGGCCAATCGTCTGACCTGCCTCAACCTCCTGTCAATGCTGGCGGCG |
| | GCTCTGGTGGTGGTTCTGGTGGCGGCTCTGAGGGTGGTGGCTCTGAGGGTGGCGGTTCTGAGGGTGGCGGCTC |
| | TGAGGGAGGCGGTTCCGGTGGTGGCTCTGGTTCCGGTGATTTTGATTATGAAAAGATGGCAAACGCTAATAAG |
| | GGGGCTATGACCGAAAATGCCGATGAAAACGCGCTACAGTCTGACGCTAAAGGCAAACTTGATTCTGTCGCTA |
| | CTGATTACGGTGCTGCTATCGATGGTTTCATTGGTGACGTTTCCGGCCTTGCTAATGGTAATGGTGCTACTGG |
| | TGATTTTGCTGGCTCTAATTCCCAAATGGCTCAAGTCGGTGACGGTGATAATTCACCTTTAATGAATAATTTC |
| | CGTCAATATTTACCTTCCCTCCCTCAATCGGTTGAATGTCGCCCTTTTGTCTTTGGCGCTGGTAAACCATATG |
| | AATTTTCTATTGATTGTGACAAAATAAACTTATTCCGTGGTGTCTTTGCGTTTCTTTTATATGTTGCCACCTT |
| | TATGTATGTATTTTCTACGTTTGCTAACATACTGCGTAATAAGGAGTCTTAATCATGCCAGTTCTTTTGGGTA |
| | TTCCGTTATTATTGCGTTTCCTCGGTTTCCTTCTGGTAACTTTGTTCGGCTATCTGCTTACTTTTCTTAAAAA |
| | GGGCTTCGGTAAGATAGCTATTGCTATTTCATTGTTTCTTGCTCTTATTATTGGGCTTAACTCAATTCTTGTG |
| | GGTTATCTCTCTGATATTAGCGCTCAATTACCCTCTGACTTTGTTCAGGGTGTTCAGTTAATTCTCCCGTCTA |
| | ATGCGCTTCCCTGTTTTTATGTTATTCTCTCTGTAAAGGCTGCTATTTTCATTTTTGACGTTAAACAAAAAAT |
| | CGTTTCTTATTTGGATTGGGATAAATAATATGGCTGTTTATTTTGTAACTGGCAAATTAGGCTCTGGAAAGAC |
| | GCTCGTTAGCGTTGGTAAGATTCAGGATAAAATTGTAGCTGGGTGCAAAATAGCAACTAATCTTGATTTAAGG |
| | CTTCAAAACCTCCCGCAAGTCGGGAGGTTCGCTAAAACGCCTCGCGTTCTTAGAATACCGGATAAGCCTTCTA |
| | TATCTGATTTGCTTGCTATTGGGCGCGGTAATGATTCCTACGATGAAAATAAAAACGGCTTGCTTGTTCTCGA |
| | TGAGTGCGGTACTTGGTTTAATACCCGTTCTTGGAATGATAAGGAAAGACAGCCGATTATTGATTGGTTTCTA |
| | CATGCTCGTAAATTAGGATGGGATATTATTTTTCTTGTTCAGGACTTATCTATTGTTGATAAACAGGCGCGTT |
| | CTGCATTAGCTGAACATGTTGTTTATTGTCGTCGTCTGGACAGAATTACTTTACCTTTTGTCGGTACTTTATA |
| | TTCTCTTATTACTGGCTCGAAAATGCCTCTGCCTAAATTACATGTTGGCGTTGTTAAATATGGCGATTCTCAA |
| | TTAAGCCCTACTGTTGAGCGTTGGCTTTATACTGGTAAGAATTTGTATAACGCATATGATACTAAACAGGCTT |
| | TTTCTAGTAATTATGATTCCGGTGTTTATTCTTATTTAACGCCTTATTTATCACACGGTCGGTATTTCAAACC |
| | ATTAAATTTAGGTCAGAAGATGAAATTAACTAAAATATATTTGAAAAAGTTTTCTCGCGTTCTTTGTCTTGCG |
| | ATTGGATTTGCATCAGCATTTACATATAGTTATATAACCCAACCTAAGCCGGAGGTTAAAAAGGTAGTCTCTC |
| | AGACCTATGATTTTGATAAATTCACTATTGACTCTTCTCAGCGTCTTAATCTAAGCTATCGCTATGTTTTCAA |
| | GGATTCTAAGGGAAAATTAATTAATAGCGACGATTTACAGAAGCAAGGTTATTCACTCACATATATTGATTTA |
| | TGTACTGTTTCCATTAAAAAAGGTAATTCAAATGAAATTGTTAAATGTAATTAATTTTGTTTTCTTGATGTTT |
| | GTTTCATCATCTTCTTTTGCTCAGGTAATTGAAATGAATAATTCGCCTCTGCGCGATTTTGTAACTTGGTATT |
| | CAAAGCAATCAGGCGAATCCGTTATTGTTTCTCCCGATGTAAAAGGTACTGTTACTGTATATTCATCTGACGT |
| | TAAACCTGAAAATCTACGCAATTTCTTTATTTCTGTTTTACGTGCAAATAATTTTGATATGGTAGGTTCTAAC |
| | CCTTCCATTATTCAGAAGTATAATCCAAACAATCAGGATTATATTGATGAATTGCCATCATCTGATAATCAGG |
| | AATATGATGATAATTCCGCTCCTTCTGGTGGTTTCTTTGTTCCGCAAAATGATAATGTTACTCAAACTTTTAA |
| | AATTAATAACGTTCGGGCAAAGGATTTAATACGAGTTGTCGAATTGTTTGTAAAGTCTAATACTTCTAAATCC |
| | TCAAATGTATTATCTATTGACGGCTCTAATCTATTAGTTGTTAGTGCTCCTAAAGATATTTTAGATAACCTTC |
| | CTCAATTCCTTTCAACTGTTGATTTGCCAACTGACCAGATATTGATTGAGGGTTTGATATTTGAGGTTCAGCA |
| | AGGTGATGCTTTAGATTTTTCATTTGCTGCTGGCTCTCAGCGTGGCACTGTTGCAGGCGGTGTTAATACTGAC |
| | CGCCTCACCTCTGTTTTATCTTCTGCTGGTGGTTCGTTCGGTATTTTTAATGGCGATGTTTTAGGGCTATCAG |
| | TTCGCGCATTAAAGACTAATAGCCATTCAAAAATATTGTCTGTGCCACGTATTCTTACGCTTTCAGGTCAGAA |
| | GGGTTCTATCTCTGTTGGCCAGAATGTCCCTTTTATTACTGGTCGTGTGACTGGTGAATCTGCCAATGTAAAT |
| | AATCCATTTCAGACGATTGAGCGTCAAAATGTAGGTATTTCCATGAGCGTTTTTCCTGTTGCAATGGCTGGCG |
| | GTAATATTGTTCTGGATATTACCAGCAAGGCCGATAGTTTGAGTTCTTCTACTCAGGCAAGTGATGTTATTAC |
| | TAATCAAAGAAGTATTGCTACAACGGTTAATTTGCGTGATGGACAGACTCTTTTACTCGGTGGCCTCACTGAT |
| | TATAAAAACACTTCTCAGGATTCTGGCGTACCGTTCCTGTCTAAAATCCCTTTAATCGGCCTCCTGTTTAGCT |
| | CCCGCTCTGATTCTAACGAGGAAAGCACGTTATACGTGCTCGTCAAAGCAACCATAGTACGCGCCCTGTAGCG |
| | GCGCATTAAGCGCGGCGGGTGTGGTGGTTACGCGCAGCGTGACCGCTACACTTGCCAGCGCCCTAGCGCCCGC |
| | TCCTTTCGCTTTCTTCCCTTCCTTTCTCGCCACGTTCGCCGGCTTTCCCCGTCAAGCTCTAAATCGGGGGCTC |
| | CCTTTAGGGTTCCGATTTAGTGCTTTACGGCACCTCGACCCCAAAAAACTTGATTTGGGTGATGGTTCACGTA |
| | GTGGGCCATCGCCCTGATAGACGGTTTTTCGCCCTTTGACGTTGGAGTCCACGTTCTTTAATAGTGGACTCTT |
| | GTTCCAAACTGGAACAACACTCAACCCTATCTCGGGCTATTCTTTTGATTTATAAGGGATTTTGCCGATTTCG |
| | GAACCACCATCAAACAGGATTTTCGCCTGCTGGGGCAAACCAGCGTGGACCGCTTGCTGCAACTCTCTCAGGG |
| | CCAGGCGGTGAAGGGCAATCAGCTGTTGCCCGTCTCACTGGTGAAAAGAAAAACCACCCTGGCGCCCAATACG |
| | CAAACCGCCTCTCCCCGCGCGTTGGCCGATTCATTAATGCAGCTGGCACGACAGGTTTCCCGACTGGAAAGCG |
| | GGCAGTGAGCGCAACGCAATTAATGTGAGTTAGCTCACTCATTAGGCACCCCAGGCTTTACACTTTATGCTTC |
| | CGGCTCGTATGTTGTGTGGAATTGTGAGCGGATAACAATTTCACACAGGAAACAGCTATGACCATGATTACGA |
| | ATTCGAGCTCGGTACCCGGGGATCCTCAACTGTGAGGAGGCTCACGGACGCGAAGAACAGGCACGCGTGCTGG |
| | CAGAAACCCCCGGTATGACCGTGAAAACGGCCCGCCGCATTCTGGCCGCAGCACCACAGAGTGCACAGGCGCG |
| | CAGTGACACTGCGCTGGATCGTCTGATGCAGGGGGCACCGGCACCGCTGGCTGCAGGTAACCCGGCATCTGAT |
| | GCCGTTAACGATTTGCTGAACACACCAGTGTAAGGGATGTTTATGACGAGCAAAGAAACCTTTACCCATTACC |
| | AGCCGCAGGGCAACAGTGACCCGGCTCATACCGCAACCGCGCCCGGCGGATTGAGTGCGAAAGCGCCTGCAAT |
| | GACCCCGCTGATGCTGGACACCTCCAGCCGTAAGCTGGTTGCGTGGGATGGCACCACCGACGGTGCTGCCGTT |
| | GGCATTCTTGCGGTTGCTGCTGACCAGACCAGCACCACGCTGACGTTCTACAAGTCCGGCACGTTCCGTTATG |
| | AGGATGTGCTCTGGCCGGAGGCTGCCAGCGACGAGACGAAAAAACGGACCGCGTTTGCCGGAACGGCAATCAG |
| | CATCGTTTAACTTTACCCTTCATCACTAAAGGCCGCCTGTGCGGCTTTTTTTACGGGATTTTTTTATGTCGAT |
| | GTACACAACCGCCCAACTGCTGGCGGCAAATGAGCAGAAATTTAAGTTTGATCCGCTGTTTCTGCGTCTCTTT |
| | TTCCGTGAGAGCTATCCCTTCACCACGGAGAAAGTCTATCTCTCACAAATTCCGGGACTGGTAAACATGGCGC |
| | TGTACGTTTCGCCGATTGTTTCCGGTGAGGTTATCCGTTCCCGTGGCGGCTCCACCTCTGAAAGCTTGGCACT |
| | GGCCGTCGTTTTACAACGTCGTGACTGGGAAAACCCTGGCGTTACCCAACTTAATCGCCTTGCAGCACATCCC |
| | CCTTTCGCCAGCTGGCGTAATAGCGAAGAGGCCCGCACCGATCGCCCTTCCCAACAGTTGCGCAGCCTGAATG |
| | GCGAATGGCGCTTTGCCTGGTTTCCGGCACCAGAAGCGGTGCCGGAAAGCTGGCTGGAGTGCGATCTTCCTGA |
| | GGCCGATACTGTCGTCGTCCCCTCAAACTGGCAGATGCACGGTTACGATGCGCCCATCTACACCAACGTGACC |
| | TATCCCATTACGGTCAATCCGCCGTTTGTTCCCACGGAGAATCCGACGGGTTGTTACTCGCTCACATTTAATG |
| | TTGATGAAAGCTGGCTACAGGAAGGCCAGACGCGAATTATTTTTGATGGCGTTCCTATTGGTTAAAAAATGAG |
| | CTGATTTAACAAAAATTTAATGCGAATTTTAACAAAATATTAACGTTTACAATTTAAATATTTGCTTATACAA |
| | TCTTCCTGTTTTTGGGGCTTTTCTGATTATCAACCGGGGTACATATGATTGACATGCTAGTTTTACGATTACC |
| | GTTCATCGATTCTCTTGTTTGCTCCAGACTCTCA |

TABLE 2
Staple sequences used for the octahedron triangle (T_octa) folding

| SEQ ID | |
|---|---|
| NO: | |
| | Core structure oligo sequences (5′-3′) |
| 2 | CACGTTGAGGAATTGCGAATAATCAGATGATGAATATAC |
| 3 | AGGGAAGAAAGCGAAAGGAGCGGGCTGCGGCTCGTTAGATAAAGGGA |
| 4 | CGGGGAAAGCCGGCGAACGTGGCGGAGCTCGATTGCTTTG |
| 5 | CAGGCGCAGACGGTCAAACGTAACTGGCAGCCTCCGGCCA |
| 6 | CCCTAAAGGGAGCCCCCGATTTATTCCTGTGTGAAATT |
| 7 | AATTTCAACTTTAATCTTAATAAATTTTTCGAACTA |
| 8 | GGTTTTGAAGCCTTAAAACGCTAATTTTTGAGCGTC |
| 9 | ATGGAAACAGTACATATTAGATTAGGTGCTGGTAATTTTC |
| 10 | TAGAAAATACATACATAAAGGTGGGTATTCTAGAAGGTAA |
| 11 | ATAGCCGAACAAAATAGCTATCTTACCGAAGAATGGAAACAAATATTGATATA |
| 12 | CCAACAGGTCAGGATTAGAGAGTGTACAGACTCATTCCAAGTAGATT |
| 13 | TCTATCAGGGCGTAATGAGTGTTGCAGCCCTTCACCCATTTTGAAAAACGCT |
| 14 | ATGGCCCAAACGTGGACTCCAACGCAGCACAGACAATATT |
| 15 | GACTCCTTATTACGCAGTATGTTCTCCCGACTTGCGGG |
| 16 | CGAAAGACTTCAAATATCGCGTTGGGCTTGAGATGGTT |
| 17 | GAATAAGTTTATTCGAGAATGACCATAAATCAAAAATCAG |
| 18 | AGAAACAATAAAATTAATTTTTCGTTGTAGCAGCCTGAGTAGAAGAACTC |
| 19 | GCGATTATAAATGGTCAATACCGCCAGCCAGTATCGGCC |
| 20 | CGGAGATTTGTATTACACGAAAGAGGCAAAAGACCTCCGGCTTAGGT |
| 21 | AAAGACTTTTTCATGAGGAAGAAAATACCACAAAAATAGCGAGAGGCT |
| 22 | CCGACAATGAGCGACATTTTTAATCAAGTTTATCGGCATTTTCGGTCAT |
| 23 | ACTGCCCGCTTTCCAGAGCAGTTGGGAAAAAGAGACG |
| 24 | CATTCCAAGAACGCAACCATCCTAATTTACGAAAAGCCTGTTTAGTA |
| 25 | AGAACGCGCCTGTTTATATCCTGACCCAATCCATTAACTGAACACCCT |
| 26 | AGGCGCATAGATAAGGCTTGCCAGCAAACTAGCTTAAT |
| 27 | CGGTCGTTCGTGTGATAAATAAGGCGTTAAATAAGAATAAAGCCCACGCATAACCGTGA |
| 28 | TTTGATTAGTGCCAAGCGAAACGTACAGCTTGAGAAGAGTCAATAGTG |
| 29 | TGCCAACGTTTTTCAGCACCGATCAAACTTAAATTTAATCGGCC |
| 30 | TCAGCGTAGACGCTGAAAACATAGCGATAGCAAT |
| 31 | AATTTATCTTTAGTGAGTCACCTGTTTAGCTCACGACGTGGTGGAGC |
| 32 | AAGGCACCTTTTTACCTAAAAGAGGCTTTGAGGACTACGGAACA |
| 33 | AGCTTCAAAGCGAACCAGACCGGACTGACGAGAATATGCA |
| 34 | CGCCACGGGAACAATTCTTTTTACTAATAGTACAAGGCAAAGAATTAGCA |
| 35 | CAAGAAAATTTTTTAATATCCATGTTCAGCTAATGCTTTCCAGA |
| 36 | TTACCTGAGCAGAGGCGAATTATTCAG |
| 37 | CTGCTCATTTGCCGCCTCGGGAAAGTTTTTCTAAAATCCTGTTTGATG |
| 38 | TCGTCGTATGTTTTTTAGTGTGTCCATCACGCACGGACCGAGTA |
| 39 | AAAATCCCGACTTTCTCCTTAGAAATCACTTATACTTC |
| 40 | AACTTTAATCCTTTTTATCGGAAAAGGTGGCATCGGATTTGGGG |
| 41 | TTAATTTCATACAGGGTAGCATTAACATCCAAACAGCTATATATT |
| 42 | ATCCTGAGGCTTGCAGGGAGCCGGAAACGTCGCTTGCTTATAGTTGCG |
| 43 | CAACATGTCGTTTTCGCCTTTAGCGTCAGAAGTAACAGCAAGTTAC |
| 44 | TGTAAATGGTTTGAAATACCGACCTGACCTAAATTTAATGCTG |
| 45 | CAGAGGCATTTTTTTTATAATCAAGGTTTAAACATCGGG |
| 46 | GCTCTCACGGCGGTTGGCAGCAACCGCAAGAACTTTTTTA |
| 47 | AAGGGATAGGCAACAGTTGCGCTCTAAAGCCTGGGGTGCC |
| 48 | CCGTGGTGCAACAGGACGCTCAATAGTTGGCATCTAAA |
| 49 | GTGTTCAGCAAATGCGGTCGGTGGTGCCATCCATTTCATTTGAATTA |
| 50 | GGCCATCGCCTGATAACAAGACAAAGAATTTTTGCGAGAA |
| 51 | GCAACATCAGTTGACATTATTGAACGAGTAGTAAATTTTAATTCGGGGGTAAT |
| 52 | CAGCGAAAACATAACGCCAAAAGGTAACCCTCCACCATCAAATGCCGG |
| 53 | AAAGGTATTAAACCAACAACAGTAGGGCTTTTTTAATTGAGAATCGAGCCA |
| 54 | ACAATAACAGCCAGCCTAATTAGGCGTTTTAGCGAACAGCAAACGCGCTAATA |
| 55 | AAAGGTAATTTGTTTAACGTCAAACATAAAAATATTCACA |
| 56 | ACACGTTAACGGCATCAATAACCTTGCTTTTTTCTGTAAA |
| 57 | CAGAAGTATTGGGAACGCGCGTCATGGTCATAGCTGTGAGCTTGACTTATAAA |
| 58 | TTTCACCATTTACATTGGTTTTTAGATTCACCCGGTTTGC |
| 59 | CTGATTGCAAGCGGTCTAAGAATACGTGGAAAGGAAGGTT |
| 60 | GCGAACGTATAACAAAGATTGTTCATATGTACCCCGGTTGATAAT |
| 61 | GCTCCATGAAATGCAACATAAAGCTTTTTTAATCGGTTGTACCAAAAA |
| 62 | AGATTTTTTTAGGAAATCTACGATTGTGAATTACCTTAAGAAGCAA |
| 63 | AGCGGTGCAGTCACACTCCAGAACATTTTTTATTACCGCCAGCCATTG |
| 64 | TTTGCCATCAACATGTTTTAAAACACCAACAGGTAGTTACTTAG |
| 65 | GCAAGCCGCAGAACCACTTTTCATATTTTTTCAAAATCACCGGAACCA |
| 66 | GGTAATTCGATTGAGGGAGGAGAACGCGTGCCAGTTTTTTTATT |
| 67 | CGTTCCGGGACCCCCAAAAATCATACCGGAAACAATCGGCTT |
| 68 | AGGCTCCAAAAGGACCATCAAGAGAAGGATACCGCCAC |
| 69 | TTATCCGCTCACAATTGCCAGCTGTTTTTATTAATG |
| 70 | ATAATACATTTGAGGAAGCAGCAATATTAATTTTTTAGACAGGAATAC |
| 71 | GTCACCGAAAATTGTCACAATCAATAGAAAATGCCCGTAAACCTATT |
| 72 | TATCACCTATTTCGGTAAACAGTTAATGCCTTCTAGCT |
| 73 | AGGTGAATAATTAGAGCATAGTTAGACGTTAGTAAATTTTA |
| 74 | ATATCTTTAGGAGCACTTCTGACCATGGATTAGTGAGACG |
| 75 | GGCAAAAGCGCGTACTATGGATTCGTAAGGGAGAGGCGGTGCCC |
| 76 | GAGTGTTGGGGGTCGAGGTGCCGTAAAGCACTAAATCGGA |
| 77 | TTCCAGTTTGGAACAATCTTTAATTAGAACCCTAACAACTTTTTAATAGA |
| 78 | TATTAAAGCTACGTGAACCATCACCCAAATCACCGGAAGCAGCAGGCG |
| 79 | CGCCTTTTTGGGTGCCTGTCGTCCACACAACATACGAGAGTTTTTT |
| 80 | CGACCTTTTGATAAGAGGTCATTCATGTCAAATAAGCAA |
| 81 | TGCTGAATAGAGAATCTGCCTGAGGAGTAACATGTTAA |
| 82 | AGTAAAATGTTTTTTTTGACTGGATAGCGTTTTGCTTTTTAAAGAAGT |
| 83 | CCAATACTAGCGGATTGCATCAAAAAGATTAAGAGGAAGC |
| 84 | GCGGAATCGTCATAAATGAGAGATGAGAAAGGCATTAAATTTTTGTGAGC |
| 85 | GAATCCCCGTCTTTACCCTGACTATTATAGTCTGCGATTTTAAAAACC |
| 86 | CTCAAATGCTTTAAACGATAAATTATATGATAGTCTGGCC |
| 87 | AGTTCAGAAAGAGCAACACTATCAAATTACGAAAGCCTTTCCCTGTAA |
| 88 | TTTTGCGGAGCCAGTAAAACAGGAGGCCGATATCAGAG |
| 89 | AACAAAGATTGAGTAACAACTCGTTATTAGACTTTACAAACGAGAGGG |
| 90 | GACAAAAGAGTTTTAACCACCAGAGGTTTAGTTAGGAT |
| 91 | TCAGAGAGATAATTTTTCCACAAGAATTGAGAACATTTTTAGTCAGAG |
| 92 | GTTAAGCCTAACGGAATACCCAAAAGAACTGGCATGATTA |
| 93 | CAATAATAAGAGCAAGTCCAGTAAGGCAGGTCCACCGTATTTTTTCAGGA |
| 94 | AATTAAGCAATAGGGTTTTCCCAGTATATTTTCATAACCTCAGGTCTG |
| 95 | AGCCCTTTTTTACAGAGAGAATAAAATGAAAACCGCCACCACCGGAAC |
| 96 | TTAGGCAAGTGTAGCGGTCACGCGGCGGTCATGAGAGCC |
| 97 | TCAAAAGAATAGTTTTTCCGAGATAGGGTTGTGGTTTTTTCCGAAATC |
| 98 | TTTGAATGGCTATTAGGAGTCCACTTTGCCCCATAAAGTG |
| 99 | ATTGCCTGAGAGTCTGGAGCAAACAATAATGCTGTAGCGAG |
| 100 | TGTTGGGAAGGGCGTTTTTTCGGTGCAGGGGGATGTGCTG |
| 101 | ATGATACAGGAGTGTACTGGTAATAGGCGACATTCAACGAG |
| 102 | AAGCGCAGTCTCTGAATTTACCGTAAACAATGCGCATTAGACGGGAGA |
| 103 | CGAACCACGACCAGTATTAGAGCCAACCCT |
| 104 | AAACATCGCCATTAAAAATACCGAAGCGCCGCTACAGGTCC |
| 105 | CGTTAATAGATGAACGGTAATCGTAAAACTAGTTTGCGGATGGAAGTT |
| 106 | TAATGTGTAGGTAAAGATTTTTTCAAAAGGGTCTACAAAGGCTATCAGGT |
| 107 | AAGGCGATTAAGTTCAGCGCATTAAATTTTACCCGTCG |
| 108 | TTCAACCGGCGGGAGGGCATAGTCTTTTGCGGGATCGTC |
| 109 | GTATTAAGCGGGGTCAGTGCCTTGAGTAACAG |
| 110 | GCCGCCGCCAGCATTGATTTTTAGGAGGTTGAGCGTCATACATGGCTTTT |
| 111 | AGACGATTTCAGAGCCAACGATTTAGTAATTCTGTCCAGACGACG |
| 112 | AATCCTCACTCAGAGTAGCAGCCATAAGAGAATATAAAG |
| 113 | TGCCACGCGTATTAACACCGCCTTTAAAGCC |
| 114 | TCTAAAGCCAGCAGAAGATAAAACAGAGGTGATGCGCGTATGCTTTCC |
| 115 | ATAAAAGGGACATTCTGTTTTTCCAACAGAGAGCGCGAACTGATAGCCCT |
| 116 | ATACCTAGCCTGGCCGAGAGATAGTAAAAAAAAAAGAACG |
| 117 | GACGACAGCTTTCCGGCACCGTAACGCCAAAGGAAAGATACA |
| 118 | ATTTCAACACCAATATTCCTGTAGCCAGCTTTCATCAA |
| 119 | GCGTAACGACAAACTACGCCACCCTCAGAGCCTAATTCGC |
| 120 | TTAAAAGTAACCACCAGAAGGAAAGAAATTGCGGTCGGTACGCCAGAA |
| 121 | ATCGCACTGAATTTGTCCAATTCTACTGACCAACTTTCTAACTCACAT |
| 122 | CCTCAGAGTTTTGTTAAAATTTTTATTGTAAAACGGTGTCTGGCTTAG |
| 123 | TTTTGTCGAAAGGCCGCTTGAGCCATTTGGG |
| 124 | ATTGGCGCTCCAGTAGCACCATTACCATTAGCAAGGTTTCTTTCCA |
| 125 | TTTGCCATAGGCTGAGACTCCCAGACATGAAAGGAAATTA |
| 126 | TCAGCTCATTTTTTAGCAAGGATAGTCAAATGTTTACCA |
| 127 | GGAACGCCATCAAAAATACGCCAGTACTTTTAATAAATC |
| 128 | TGGTAATAATCACCTTGCTGACGTATGAAAAAGTATAACGACCACCAC |
| 129 | AGCGGGGTTTTGCTCCGCCACCCGGCCTTGACAGGGAAG |
| 130 | GCCCCCTTATTATATCGGTTTATCAACCAATGACAACCATCCACCGGA |
| 131 | AACTATCGGCCTTGCGTAGATTTTCGTGAGGCCATTCGCCTAACAAAA |
| 132 | AGTACCAAGTATAGCCCGGAATAGGTGTAT |
| 133 | AGAATAGAAAGGAATTTTTAACTAAAAAATCTCCAAAAAA |
| 134 | AATCCTGATTGTTTTTTTTGATTATAATATCAAAATTATT |
| 135 | AACCTACCCTTCTGAATAATGGACATTCGCCGACGGCCA |
| 136 | GCACGTAAAACAGAGAAACCTCAAATATCAGTCAATAG |
| 137 | AATCAATATCTGGTCCGTCTGAATGAAAGCGCACGCTGG |
| 138 | AAATCAACAGTTGAAAAGGGTTAGCATGGAAGTAATAAC |
| 139 | ATAGGTCATCAGGAAGAAACAGGGTTGATTC |
| 140 | CGTTGGTGGATTGACCGTAATGGGGGAATTGAAGCCCCAA |
| 141 | CCTCAGAACCGCCACCATTCTGAAACAGCCCTCCAGCA |
| 142 | CTCAGAACCAACGCCTGTAGCATTGCCGAATTTTCTGTATGCGGAGTG |
| 143 | CCCAATAGGAACCCATGTAGCTAAACAACCACCCTATCTAAAGCCCTGCC |
| 144 | TTTCGTCACCAGTCATTTTCAGGGATAGCAAG |
| 145 | GGCGGATAAGTGCCGTAATTTTTTCGCCTCCCTGTAGCG |
| 146 | GATTCTCCGTGGGAACATATTTAAGAGGGGACTAGTTTGACCATTATC |
| 147 | AAACGGCGTAGACCGTGCATCTGCGGGCTTCTGGTGCCGGTGCGCAAC |
| 148 | CAATTCGACATTATCAGCAACAGCGGGAGCT |
| 149 | TTTAGAAGATTAAATCCTTTGCCCAATAGCGGAATTATCAATCAATAT |
| 150 | TATTTTTTTTTTATATCTTACCATCAAGATTAGTTGCTCGCAATAA |
| 151 | AAAACACTGCGAAACAAAGTACAACCGGAACGCGCGACCTACTAAAGT |
| 152 | AAATAAGAACCACCCTCATTTTTAGCCGCCACACAAAATA |
| 153 | TTCAACTATTAGAACCCTTTTTTATATATTTTAAAGATTC |
| 154 | AAATCACATCTAGGAATCTAGAAGGCTTATCCGCAACATATGCGCCAAA |
| 155 | CTGGCGAAGGGCCTCTTCGCTATACTTTCAAAATTTCTT |
| 156 | TCAAAGGGCTGAGAGAGAGGAAAGAGGACAGATTGACAAG |
| 157 | TTTCGCACCAAGCCATCTTTCAAACGCGGTCCGTTTTTTAAAT |
| 158 | TAATGGACTTGTGTTAAACGATGCTGATTGCGAGCACATAGGCGGCCGGAACCGA |
| 159 | TGGTCGTCTCGTCGCAAAGCTGCTCAATTT |
| 160 | TCTCTTCAGTGAGCTGGCTGACCTTCATCAACCCAAATCATCATAAG |
| 161 | TATAAACCGGATATTCATTAGAGTAATCTGAACGGTACCTTTAATTGCTAAAACCG |
| 162 | TCCGCCATGTTTACCATGAAGGGTAGCCGCACCCTCATAACGGAACGTGCCAATT |
| 163 | CGCTACCGTAATCAGTACAAAACCATCGAAACCAATCT |
| 164 | TTCATATGTTCATTAAATCAGATAATTACCGCGCCCAT |
| 165 | TATACGTAATGCCACTACGGGGTTATATAACTATA |
| 166 | TCGGCTGTCTTTCCTTATTTCATCGGAGAACAAATATTGAC |
| 167 | CCGGAGACAAAAATTTATGCAGATGACAGCATCCATTAAACGT |
| 168 | TATACCAGTCAGGACGTTGGGAAGTTTCGGAACGAGGGTA |
| 169 | AGAGGGTAGCTATTTTTATTCATTGACGACGATAAGAACTGGT |
| 170 | GTTTACCAAAAAGAAACGCAAAGACACCACGGCAAGCAA |
| 171 | TTTTTTAAGTCCTGAACATATGCGTTATACAAATTCTTACCATTTCAA |
| 172 | AATTTAGGAAAATCGCGCAAAAGAAGATGATGAAACAAACGCTGGTAATGGTTTTT |
| 173 | TTTTTCACTCTGTGGTGCGCTAGGGCGCTAGAAAAGTAAGCAG |
| 174 | TTTTTGTAAAGGTTTCGCGCGCCTGTGTTTTT |
| 175 | TTTTTAGCTACAATTTTCAACAATAGATTTTT |
| 176 | AAATAGCAGTTACCAGAAGGAAACCGAGGAAAATTTTGCACCCTTTTT |
| | H1 design oligo sequences (5′-3′) |
| 177 | GCAGTAGAGTAGGTAGAGATTAGGCACAATACTATTAATTCTGGTCATGTACATCGACATAAATAATTGCG |
| 178 | GCAGTAGAGTAGGTAGAGATTAGGCATCAGATGTAAAACATTCAGGCAAACCAGGCAAAGCGC |
| 179 | GCAGTAGAGTAGGTAGAGATTAGGCAAGAGACTACCTTTTTAAATACACTCGCGAGCTATTGTGTC |
| 180 | GCAGTAGAGTAGGTAGAGATTAGGCAATGCATTCAAATATATTTTAGACCCTCAGCATTATGA |
| 181 | GCAGTAGAGTAGGTAGAGATTAGGCATACCGTCGAGGTGCAGTTTCAGGGATTTTCCGTAACACTGAG |
| 182 | GCAGTAGAGTAGGTAGAGATTAGGCAATCATAATTACTAGAAGCATGTAGATAGCAGCGTACCGCATATAA |
| 183 | GCAGTAGAGTAGGTAGAGATTAGGCACCATATTTAACAACGCTACCGACAGAGCCACCCTCAGAAC |
| 184 | GCAGTAGAGTAGGTAGAGATTAGGCACTTTTCGTCAGATGGCAATTCTCATATTCCTGATTAT |
| 185 | GCAGTAGAGTAGGTAGAGATTAGGCATTAATTACATTTAACACACGCAACAAAGAGTCAGATGCCG |
| | H2 design oligo sequences (5′-3′) |
| 186 | GCAGTAGAGTAGGTAGAGATTAGGCATTTTTTCAATACTATTAATTCTGGTCATGTACATCGACATAAATA |
| | ATTGCG |
| 187 | GCAGTAGAGTAGGTAGAGATTAGGCATTTTTTTTCAGATGTAAAACATTCAGGCAAACCAGGCAAAGCGC |
| 188 | GCAGTAGAGTAGGTAGAGATTAGGCATTTTTTTTTTTTTAGAGACTACCTTTTTAAATACACTCGCGAGCT |
| | ATTGTGTC |
| 189 | GCAGTAGAGTAGGTAGAGATTAGGCATTTTTTTTATGCATTCAAATATATTTTAGACCCTCAGCATTATGA |
| 190 | GCAGTAGAGTAGGTAGAGATTAGGCATTTTTTTTACCGTCGAGGTGCAGTTTCAGGGATTTTCCGTAACAC |
| | TGAG |
| 191 | GCAGTAGAGTAGGTAGAGATTAGGCATTTTTTTTATCATAATTACTAGAAGCATGTAGATAGCAGCGTACC |
| | GCA |
| 192 | GCAGTAGAGTAGGTAGAGATTAGGCATTTTTTTTTTATAACCATATTTAACAACGCTACCGACAGAGCCAC |
| | CCTCAGAAC |
| 193 | GCAGTAGAGTAGGTAGAGATTAGGCATTTTTTTTTCTTTTCGTCAGATGGCAATTCTCATATTCCTGATTA |
| | T |
| 194 | GCAGTAGAGTAGGTAGAGATTAGGCATTTTTTTTTTTTAATTACATTTAACACACGCAACAAAGAGTCAGA |
| | TGCCG |
| | H3 design oligo sequences (5′-3′) |
| 195 | GCAGTAGAGTAGGTAGAGATTAGGCATTGCAGTAGAGTAGGTAGAGATTAGGCACAATACTATTAATTCTG |
| | GTCATGTACATCGACATAAATAATTGCG |
| 196 | GCAGTAGAGTAGGTAGAGATTAGGCATTGCAGTAGAGTAGGTAGAGATTAGGCATCAGATGTAAAACATTC |
| | AGGCAAACCAGGCAAAGCGC |
| 197 | GCAGTAGAGTAGGTAGAGATTAGGCATTGCAGTAGAGTAGGTAGAGATTAGGCAAGAGACTACCTTTTTAA |
| | ATACACTCGCGAGCTATTGTGTC |
| 198 | GCAGTAGAGTAGGTAGAGATTAGGCATTGCAGTAGAGTAGGTAGAGATTAGGCAATGCATTCAAATATATT |
| | TTAGACCCTCAGCATTATGA |
| 199 | GCAGTAGAGTAGGTAGAGATTAGGCATTGCAGTAGAGTAGGTAGAGATTAGGCATACCGTCGAGGTGCAGT |
| | TTCAGGGATTTTCCGTAACACTGAG |
| 200 | GCAGTAGAGTAGGTAGAGATTAGGCATTGCAGTAGAGTAGGTAGAGATTAGGCAATCATAATTACTAGAAG |
| | CATGTAGATAGCAGCGTACCGCA |
| 201 | GCAGTAGAGTAGGTAGAGATTAGGCATTGCAGTAGAGTAGGTAGAGATTAGGCATATAACCATATTTAACA |
| | ACGCTACCGACAGAGCCACCCTCAGAAC |
| 202 | GCAGTAGAGTAGGTAGAGATTAGGCATTGCAGTAGAGTAGGTAGAGATTAGGCACTTTTCGTCAGATGGCA |
| | ATTCTCATATTCCTGATTAT |
| 203 | GCAGTAGAGTAGGTAGAGATTAGGCATTGCAGTAGAGTAGGTAGAGATTAGGCATTAATTACATTTAACAC |
| | ACGCAACAAAGAGTCAGATGCCG |

TABLE 3
Staple sequences used for the T1 pentamer triangle folding

| SEQ | |
|---|---|
| ID | |
| NO: | |
| | Core structure oligo sequences (5′-3′) |
| 204 | TGCGGCGGCCGGGTCAGTCCAGCATCAGCTCGATAACGGA |
| 205 | TGCAGCAAGCCTGGGGTGCCTAATGAGTGAGCTTTTTAACTCACATTAATTG |
| 206 | ATTTCATTTGAATTACCTTTTTTAAGAAGATGATTCATTT |
| 207 | GGAGGTCAATAACCTTTTTTTTTATAGTAGTAGTTTTTATTAATAGAT |
| 208 | CTCACAGTGTTTCTGCACAACTAAAGGATTTA |
| 209 | AAAAGAATTAAGTACTATGCCGTACTGGTAATAAGTTTTAACTTGCGTA |
| 210 | TACCTACATTTTGAAAGGGACGAATGGCTCGCTTAGGAGCACTACAGCACG |
| 211 | AAATAGTTTGACCATCATCCAATAA |
| 212 | GTTAGAACTCAAACTACCTGAAAGC |
| 213 | TATGACCCTGTAATACTTTATAAAGCCTCAGA |
| 214 | GCGCGAACTGATAGAAGATAATGCTGAACCTCAATTTTAAA |
| 215 | ACCACCAGAGCCGTTGATATTTGATACAGGAGTGAGTAAA |
| 216 | GCATAAAGCTAAAAGAAGCCTGTGAGAAAGGCCGATTGA |
| 217 | CGGAACAAAGAAACCAAGCCCGGATCAAGTTTCCGGTTTTGCTCAGT |
| 218 | CATTTTGTATCATCATATGGGTCGAGGTTTTTTCCGTTCATTTGC |
| 219 | AATATTTGCATTAAATTGTTTAGACTGTTTTTATAGGCATCGTA |
| 220 | GTGTACAGACCAGGCGCATAGGCTATGCCACTGAGGCGCAAAACAGCT |
| 221 | CAGCTTTCATCAACATTAAATGTGAGCGAGTCAGCTCAT |
| 222 | GCCGGAAACGTACCCCGGTTGATATATAAGCA |
| 223 | TACTCAGCCCTCAGAACAGAGAGATAATTTTTCCACACGCCAGG |
| 224 | GAGGTTTAGTACCGCCAAAACAGGGGGGCGCGCTTTAAAA |
| 225 | TTTTTAACTATTTTGTTGCTTTAAATTCACC |
| 226 | ACAGCGCCCACCGGAAACAATCGGAAGGTGCCGTCGAGAGTATCACCG |
| 227 | GTGCCAGCTGCATTAATGAATCGGCCAACGCCAGGGTGGTTTTTCTT |
| 228 | CGCTTTCCAGTCGGGAGTTAACGGTGGTGGTTACAAGAGTCCACAATCCGCCGGGC |
| 229 | CCTGTAGCCAAAAATAATTCGCGTAAATTAAATCCTTTGCAACATTAT |
| 230 | CAGAACGATCAACTTTAATCATTGAGCTCAACAGCTTCAA |
| 231 | TCACGACGTTGAGCGCTAATATCCGCAAGTCAGATTGAATA |
| 232 | GGAATTTGTGAGAGATCAGATGATGGCAATTCCCAGAAGGGGGGAAAGTTTGCCA |
| 233 | ACGGGTAAAATACGTAGGCTGACCACGTTAATGACGGTCA |
| 234 | ACGAAGGCGCGCCGACAATGACAAGCTCCAAAAGGAGCGCAATGAATT |
| 235 | AGAGCCACCACCCTCAACCCTCAGATAGCTATAGCTAGCAAGGACCAT |
| 236 | TGAGAATCGCTTTTTATATTTAACTACCTTTTTTTTTAACCTCCGG |
| 237 | CGCAACTGCGCAGAGGGAATTAACTGAACACCAAATAGCAAACCGCCA |
| 238 | CCAGCGGTGCTTTTTGGTGCCCCCGGTATTTTTTGGGTAAAGGTTT |
| 239 | TGCATCAGACGATCCATGTAAAGCGGTCCACGAATCATGG |
| 240 | CATGAGGAAGTTTTTTTCCATTAAATAATTTTTCGATATATTCGGT |
| 241 | AATAGCAAGCAAATCAGATATAGTCAAATATATCCCAATCCAAAGAT |
| 242 | ATCGTAGGAATCATTATATAAAGCGCCAGTTAATAGCAGCCTTAGACGCTGAGAAG |
| 243 | AGGCGGTTAGAAACCAATCAATACTAATTTACGAGCATG |
| 244 | CTGATGCATTAAAATTCTTACCAGCCGCGCCCGCCTTAAATCAAGATT |
| 245 | CTTAGGTTAACAGTAGGGCTTAATAGCCGTTTCCAGCTACAA |
| 246 | CTTTGCTCGCCGGGTTACCTGCAGCGTTGCGCCTGAGAGAGT |
| 247 | TTCTAAGAAGAGGACAAGAGGCAACCGCGACCTACAACGGGGCTATCA |
| 248 | AAGGCTTAGCGAACCTCCCGACTTACATTCAACAAACGTA |
| 249 | TTGCGGGAAACGAGGGTAGCAACGAGTGAATAGATTTTAAGAACTGGC |
| 250 | ATCGGCTGTCTTTCCTTATCATTCTTAGGCAGTAAGTCCTGTGAATTT |
| 251 | GTGAATAACCTTGCTTTGTAAATGAAATGAAACAAAATAAAAGGTGGC |
| 252 | CGTCGCTACAATAACGGATTCGCCCTATTACGCCAGCTGG |
| 253 | TTAATTAATTTTCCCTTTTTTAGAATCCTTGAAAACCATAGGTCTGAGAGAC |
| 254 | CGGTGGTGCTGGTCTGGTCAGCAGTAGCTCTCACGGAAAA |
| 255 | CCATCCCACGCAACCATTTTTCTTACGGCTGGAGGTCTGTTGCCCTGCGGCT |
| 256 | AGCCGGAAGCATAAAGGCGCAGTGGAATTCGTGGTATGAGGCCGTTTT |
| 257 | GAATAATACAGTTTCAGCGGAGTGGGAACAAACGGCGGAT |
| 258 | ATTTTTTCACGTTGAATTTTTATCTCCAAAAAAAAGCAACCATCGCCCACGC |
| 259 | TTCTGTATTAGGTCACGTTGGTGTAGATGGGCCCAGGCAA |
| 260 | TTTCCAGATCCAGCCATCACCAGTAAACAAGAGGTCATTG |
| 261 | GATGAATATACAGTAAGCTGCAAGGCGATTAAGTTGGGTACGAAACGT |
| 262 | ACGTGCCGAGCGGATCAAACTTAAATTTCTGCCTGGCCTT |
| 263 | AGGCTTGCCCTGACGAGAAACACCGAAAGACCACATTCAACTAATTC |
| 264 | GATCGCACCGTTAGTACTGTAGCATTCCACAGGCGATTAT |
| 265 | GCTTTCCGGTCAATCATATGTCCAATACTGCGGA |
| 266 | GTAAAACGACGGCCATTTTTTGCCAAGCTTTCAGGTTTTCCCAG |
| 267 | ACAAAGCTGCTCATTCGCTACAGAGAAAGATTAGTAAGAGCAATGCTTTCGAGGTG |
| 268 | TAACTGATTGTTTGGATTTCAGGGCGATGGCAGCTTGACAGCGGAAT |
| 269 | TGTACATCGACATAATTTTTAAAATCCCGTAAAACGCCAGCAGT |
| 270 | AACGCCAACATGTAATCAAGAACGAATCTTACGAACAAGA |
| 271 | AGGGGACGACGACAGTTTTTATCGGCCTCAGGAAACCGTGCATC |
| 272 | TTAGACGGGAGAACCCGAAGCCCTTCCTTATTTGCAGCCA |
| 273 | GTCGGGGTCATTGCAGGCGTTTTTTTTCGCACTCTA |
| 274 | GTTTTTTCCACGGTCAGGCCAGAACGCCTGTGCACTCTGTTTCCACAC |
| 275 | TGCCGTTCCGGCAAACCTTTAGTTCGACAACTCGTAGCACTAAATCGGA |
| 276 | AATTTCTTAAAACGAACTAATTTTTGGAACAACATTATCCAGTCAGTCAACGTA |
| 277 | TGATACCGCAACCTTTAATTGTATCGGTTTTTTTATCAGCTCAC |
| 278 | AGATTTGTAACACTCATAGTTAGCGTAATTATGAAACA |
| 279 | ACCAAGTTACCAACCTAAAACGAAGATGAACGACTGACCAACTTTGAA |
| 280 | AGTCAATACAACGCTAACGATTTTTCGTCTTTCCAGAGTTTTGCACTTATTTTC |
| 281 | AATATAAAATTATTTGCACGTGCGATATTTTTCTTAGATTATAC |
| 282 | CTGTCCATAATGGAAGGGTTAGGGAACGGAACCAGGCGGATAAAATTGAGTTAAGC |
| 283 | GACGACGACAAAGCCCGAGATAGGTTAATGCGGAGAAAGG |
| 284 | GTTGAGTAAAGGGCGAAAAACCGTCTAATAAACGTGGC |
| 285 | GCGGTTGCCTGGTTTGCCCCTTTTTGCAGGCGAAAATCGCCTGGCCTCACTGCC |
| 286 | TGTTCTGCAGATACATAAGAAAGACTGAGAATGA |
| 287 | CGCCAAAGACGATAAAAACCAAAATAGACGCAGAAAAC |
| 288 | CAAAGAACCCTTAAGAAACGATTTATTAAGACTTTTAAGA |
| 289 | CCTAATTTCAACGCTCGGGTTATATAACTATACTGTAAATAGAGAGAA |
| 290 | TTCAGCTAATGCAGAAAGTAATTCTGAAACAGAAGGATTACCACCGG |
| 291 | TATGCAACTAACAGTTGAAGCGAACGAAGCCC |
| 292 | AAGGAGCGCGTAACCACCACACCCACGTATAAGGCAAAATGTGAGACG |
| 293 | CATATCCAGAACAATTTTTTTTACGATAGAACCTTTTTTTCTGATCGG |
| 294 | TACAGGTAGGCTTTGACTTGCAGGGAGTTAAAGGAATTGCTATCATAA |
| 295 | AGAAAAATCTTTCATCAAGAGTAATCTTGACATTTTTGAACCGGATATTCAT |
| 296 | TTTTATCCTGGGTATTAAACCAAGTACCGCACTTTTTCATCGAGAACAAGCA |
| 297 | CCGGAATCATAATTACATTTAATGAAACTTTT |
| 298 | TGTGAAATTGTTATCCATCTGGTCGAAGGTTAGTGAGGCGACAGACAA |
| 299 | CGCTGAGGGGACTAAAGACTTTTTTACCCAAAGACGTTGGGA |
| 300 | TGATAAGAGGTCTTTTTTTTTTGCGGATGGTCATTATA |
| 301 | AATATGATGAGAGGGTAACGCAAGCAAAGAATTAGCAAA |
| 302 | CCAGCGCCCGGAAATTCGCAGTCTCTTTTTGAATTTACCGTTCCAG |
| 303 | ATGGTTTAAACATATAAAAGAAACGTTTTTAAAGACAC |
| 304 | TTTGTCACAATCTTTTTATAGAAAATTCATAGTTGCTA |
| 305 | CTGTTTGACATCAGATGTCATAAACATCCCTTAGCACCGTTTAAAGAA |
| 306 | GGGGTCAGAATGCCCCAAATAAATCTCAGAGCCACCACC |
| 307 | TGAGGCCAGTTGCTTTGTAATAACATCACGCCCCGCCAGCATTGACAGGAG |
| 308 | CTGAGAAGATTAACCGAGTGCCACGTTTTTTGAGAGCC |
| 309 | AAGGGATTTTAGTTTTTCAGGAACGGTACGCCTTCACC |
| 310 | AGTTGGCAATGAAAAATCTAAAGCATCACCTAACAGAG |
| 311 | CTTAGAGCTCCAACAGGTCAGGATTTTTTTGAGAGTAC |
| 312 | CTTTAATTTAGTCAGAAGCTTTTTAAGCGGATTGCATACCCTG |
| 313 | TAATGCCGATTCAACCTGTGTAGGTAAAGATTCAAAAGGTTATTTC |
| 314 | AGCTATTTTTGACAGAAATTGTGGCGTTTTATCCGGTA |
| 315 | TATGTTAGCCGGAGACAGTCAAATCACCATCACGCGAGTCGAAAT |
| 316 | CACGGAATAACCGAGGAAATTTTTGCAATAATAACGAGTTACC |
| 317 | AAACAGTTTGCCTTGATAAGCGTCATACATGGCTTTTGACACAAAC |
| 318 | CTGCCTATTTCGCGCACAACATGTTGGGCGCGCGGGGAG |
| 319 | GAATCAGAGTTTTTGGGAGCTAAACAGGAGGGCAAGTGTAG |
| 320 | TCTAAAATATCTGTTGCGTCCGTGTTTAATTGTAGTAAATTGGGCTGCTCACAA |
| 321 | TTAATTCGATGATATCAAACCCTCAATCAATTGAGATGGAGCCTC |
| 322 | AAGCAAACTTAATTGCGTCTGGAAATATTTTAATTTTTTGCAATGC |
| 323 | CCGCTACATTGTTGCCTGAGTAGAGAGGCAGGGCATTTTCGGT |
| 324 | GGTCACGCTGCGGGCGCTATTTTTGGCGCTATAGATAA |
| 325 | TCAAATATCCAGAACGAGTAGATTAATACCGATCGTCTGAAAT |
| 326 | ACTATTATCTGGAGCACAAACTAATAGCGCGAAACAAAGTGCTCCAT |
| 327 | GAATCGATTCTACTAAGCTATATTTTCATTTAAGATTGATCAGAA |
| 328 | CCGAACAAGAATACCCAAAAGAACCATACATAACAGCCATGTTTTGAA |
| 329 | AGAAGGACTGAGACTATATCAAAGTACCGACAAAAGGTAACGCGCCT |
| 330 | GCAACGACCAGTAATAACGCTCAAACGAACCAATTAGTCTTTAAT |
| 331 | CAGGTCTTTCAAAAAGATTAAGAGCAGACCGGGAGATTTACCTTATGC |
| 332 | CTAGCATGCAGCAAGCCCAATAGCGAACGATCTAAAGTT |
| 333 | CCAATAATAAGAGCAAGAGCAGATAGAAACAGGGAACGTCAA |
| 334 | TTCCCAATTCTGCGCAGCCCTAAAACATCGCCATT |
| 335 | CCCTCAAATAAAATTCAAATTGTAAACGTTAATTTAAAAG |
| 336 | ATCGTCATAAATATTCATTCAAAAATTACCAGACAGGAATTA |
| 337 | CGATAGCAGCACTTTTTGTAAAACCGCCTCTTTTTCTCAGAAATC |
| 338 | TCAGTAGCGACAGAAATAGGTGGGTTGAT |
| 339 | TCTTTTAATAGCCCCCTTATTAGCGGCCTTTAGCGTCA |
| 340 | CAGGAAAAACATTTACAAACAATGATGAAGACGCCAT |
| 341 | AGTCACAATTTATTTACATTGGCAGACGCTCATGGAAA |
| 342 | CATCAATGAACGGTAATCGACCATGTACCGTATCATCG |
| 343 | GCACCATTACCATTGAAAAGGTGGATTAAGCAACGGAGATCTACAAA |
| 344 | ACCAGTAAAAGTAAAACAATGCTGAACACACCCTCGGCGATC |
| 345 | CAGAGCCAGGATTAGCGGGGCGCTTCTGAA |
| 346 | GGTCATAATCAAAATCTTTCATCGTCAGACGACGCCACCAGAACC |
| 347 | CCTTGCTAAGGGAAGATTTAGCCACTACTGATTATAGACTTT |
| 348 | CCAACAGACGCCAGCCATTGCAATTTGAGTCCGAACG |
| 349 | CGTCACCGACTTGGGGTCGGTTGTACCAAAAACAT |
| 350 | ACATTTCCCATAAATGAATCCCAAAAGAGTTAAATAACAACC |
| 351 | ATCATACCCTGAGAGTGATAAATGTTACTTAGGAACCGA |
| 352 | AGGCAAGGGATAAAAATTTTTAGAACCTTTTTTCATGTTTCATT |
| 353 | TTTGGGAATTAGAGCCAGCAAGCCGCCACC |
| 354 | CTCAGAATTAAGAGGGCCCGTATGTTTATCAATCCCATC |
| 355 | CCGCCACCCCTCATTAAAGCCAGAATGTTTTTAAAGATTCATTA |
| 356 | GTAAGAAAGCCGTCAGTTGAAAGCCCGGGTAGTTTCCTGAACATACG |
| 357 | TATTTTTATTCTGGGAAGTATTAGACGTTATGCTGATCGTGCC |
| 358 | CTGAGTAAGTTCTAGCGCTCCTTTATCATAAGGCCGGAAC |
| 359 | AAGGTGAAAATATTGAAAAGACAAAAGGGCGGCGGGAG |
| 360 | GTCAGTATTAACACCGCCTTTTTTCAACTTGTAGCAATACTTCT |
| 361 | GACTGTAGCGCGTACCGGAACCTCAGAGCGGGGAACCTATTATT |
| 362 | CCATATAAAGTACGGTTGAATATAATGCTGT |
| 363 | AGAGTCTGTCCATTGATTAGACGAGCGCCGCGC |
| 364 | GGGAGGGAAGGTATTATCACGAAAATATGGCATG |
| 365 | TCACGCAATGTTTTTATAATCAGTTCACCACCCTTATAAATC |
| 366 | AGCAGCAAAATCAACAGCCGATTATCATAGCTCCGAGCTCTCACTGCG |
| 367 | TTTGTTTAAGCGCATATGTGACCAAGTTAAGTACT |
| 368 | TTATACATTTCATCTTCTGACCTAATAGAAAAATCGCAAGACAATTACC |
| 369 | TGTTAAATAAGGACCCCCAACAGCCCTGAGTTTCG |
| 370 | ATTATTTATTTTAGAATCCAAGCCTGTTTAGTATCATT |
| 371 | AAGAATACACTAAAGAGTTTGAAATACCGACCGTGTGATAAAT |
| 372 | TCAATATGAGCAAAATGGAAACCAAAATCGTTGGGAAG |
| 373 | TACATTTAACATTGTCGTCAGCGCCATCTTCTGGT |
| 374 | GCGAACACTCATCTTTAATAAACAAACATCAAGAAAACAAAAT |
| 375 | TTAATTCAGCAAATCAACCTGTCGGCAACAGCTGATTGCCCAGAATCCCTCGTTA |
| 376 | TATGCCAGTAATAAGAGATCAAAATATAAAAACAGAAATAA |
| 377 | AGGTGGAGTAACGTCAAGAAATTGCGTAGATTAATT |
| 378 | GTAACAGTAAGTTTATAAAATAATACAATAGAAGGCATTTTCGAGCGTT |
| 379 | TGGGCGGTTGATCAAGTTTTTTGTCCGTGAACCAAGAATTT |
| 380 | ACCCTAAAGGGAGCCCCCGAAAGCGACCAACGTCGTTGTTCCGTGGGCT |
| 381 | TTTAATGCCAACGGCACACTGGTAGTTTGGACCGAAATCCGTGCTTT |
| 382 | TATATTCAGGTTCCGCCACGAACCTACCCCTCAAGATGAAAGTA |
| 383 | AAAGCCGCACATCCTCTCGCTGGCAGCCTCCGTTTT |
| 384 | TGCCAGTTTGGTAATAGTAAAATTTTAGTTTTGCAAGCCAT |
| 385 | TGAATTAGGAATACAGCATCGGTCGTCACCCTCAGCAGCAGT |
| 386 | TTGCGCCAGAGCACAGGCGGCGCGGTCCTACATTTGTAGATTAGTACGTGGC |
| 387 | TACGGGTGCTGCTACCGGGGTGAGGATCGAATTGAG |
| 388 | TTAAGAACAACTAAAGGCCGCTTCGAGGCATCATCAGTT |
| | H1 design oligo sequences (5′-3′) |
| 389 | GCAGTAGAGTAGGTAGAGATTAGGCAAGAGACGTTTTTAGAAACGACTTGTAGAATTTTTGTCAGCGTGGTG |
| 390 | GCAGTAGAGTAGGTAGAGATTAGGCACGAAAGGTTTTTGGATGTCAGTACCTTTTTTTTTCATCGGGAGAAA |
| 391 | GCAGTAGAGTAGGTAGAGATTAGGCACGTCGGATTCTCCGTGAGAATAGACAGAGGGGCCCTCGTT |
| 392 | GCAGTAGAGTAGGTAGAGATTAGGCACTCCGTGGTGAAGGGACAACCGCATCACCCAACGTGGACT |
| 393 | GCAGTAGAGTAGGTAGAGATTAGGCAATAAGTATATCAATATAATCCCTATGTTTACCAGTCCC |
| 394 | GCAGTAGAGTAGGTAGAGATTAGGCATTATTAATCAATAGGAGGTAAAGTTAACGAGAGGCTTTTG |
| 395 | GCAGTAGAGTAGGTAGAGATTAGGCATGACCGTTTTTTATGGGAGGGATTTTGCTTTTTTAACAACTTTCAA |
| 396 | GCAGTAGAGTAGGTAGAGATTAGGCAGGTGCGGGCCTCTTCGTGATTGCTGGGTAATTTAACATAA |
| 397 | GCAGTAGAGTAGGTAGAGATTAGGCAAAGCCCCATTTTCAGGGATACCGTCGCCATTCAGGCTG |
| | H2 design oligo sequences (5′-3′) |
| 398 | GCAGTAGAGTAGGTAGAGATTAGGCATTTTTTTTTTTTTTTTTTTTAGAGACGTTTTTAGAAACGACTTGTA |
| | GAATTTTTGTCAGCGTGGTG |
| 399 | GCAGTAGAGTAGGTAGAGATTAGGCATTTTTTTTTTTTTTTTTTTTCGAAAGGTTTTTGGATGTCAGTACCT |
| | TTTTTTTTCATCGGGAGAAA |
| 400 | GCAGTAGAGTAGGTAGAGATTAGGCATTTTTTTTTTTTTTTTTTTTCGTCGGATTCTCCGTGAGAATAGACA |
| | GAGGGGCCCTCGTT |
| 401 | GCAGTAGAGTAGGTAGAGATTAGGCATTTTTTTTTTTTTTTTTTTTCTCCGTGGTGAAGGGACAACCGCATC |
| | ACCCAACGTGGACT |
| 402 | GCAGTAGAGTAGGTAGAGATTAGGCATTTTTTTTTTTTTTTTTTTTATAAGTATATCAATATAATCCCTATG |
| | TTTACCAGTCCC |
| 403 | GCAGTAGAGTAGGTAGAGATTAGGCATTTTTTTTTTTTTTTTTTTTATTAATCAATAGGAGGTAAAGTTAA |
| | CGAGAGGCTTTTG |
| 404 | GCAGTAGAGTAGGTAGAGATTAGGCATTTTTTTTTTTTTTTTTTTTGACCGTTTTTTATGGGAGGGATTTTG |
| | CTTTTTTAACAACTTTCAA |
| 405 | GCAGTAGAGTAGGTAGAGATTAGGCATTTTTTTTTTTTTTTTTTTTGGTGCGGGCCTCTTCGTGATTGCTGG |
| | GTAATTTAACATAA |
| 406 | GCAGTAGAGTAGGTAGAGATTAGGCATTTTTTTTTTTTTTTTTTTTAAGCCCCATTTTCAGGGATACCGTCG |
| | CCATTCAGGCTG |
| | H3 design oligo sequences (5′-3′) |
| 407 | GCAGTAGAGTAGGTAGAGATTAGGCATTGCAGTAGAGTAGGTAGAGATTAGGCAAGAGACGTTTTTAGAA |
| | ACGACTTGTAGAATTTTTGTCAGCGTGGTG |
| 408 | GCAGTAGAGTAGGTAGAGATTAGGCATTGCAGTAGAGTAGGTAGAGATTAGGCACGAAAGGTTTTTGGAT |
| | GTCAGTACCTTTTTTTTTCATCGGGAGAAA |
| 409 | GCAGTAGAGTAGGTAGAGATTAGGCATTGCAGTAGAGTAGGTAGAGATTAGGCACGTCGGATTCTCCGTG |
| | AGAATAGACAGAGGGGCCCTCGTT |
| 410 | GCAGTAGAGTAGGTAGAGATTAGGCATTGCAGTAGAGTAGGTAGAGATTAGGCACTCCGTGGTGAAGGGA |
| | CAACCGCATCACCCAACGTGGACT |
| 411 | GCAGTAGAGTAGGTAGAGATTAGGCATTGCAGTAGAGTAGGTAGAGATTAGGCAATAAGTATATCAATATA |
| | ATCCCTATGTTTACCAGTCCC |
| 412 | GCAGTAGAGTAGGTAGAGATTAGGCATTGCAGTAGAGTAGGTAGAGATTAGGCATTATTAATCAATAGGAG |
| | GTAAAGTTAACGAGAGGCTTTTG |
| 413 | GCAGTAGAGTAGGTAGAGATTAGGCATTGCAGTAGAGTAGGTAGAGATTAGGCATGACCGTTTTTTATGG |
| | GAGGGATTTTGCTTTTTTAACAACTTTCAA |
| 414 | GCAGTAGAGTAGGTAGAGATTAGGCATTGCAGTAGAGTAGGTAGAGATTAGGCAGGTGCGGGCCTCTTCG |
| | TGATTGCTGGGTAATTTAACATAA |
| 415 | GCAGTAGAGTAGGTAGAGATTAGGCATTGCAGTAGAGTAGGTAGAGATTAGGCAAAGCCCCATTTTCAGG |
| | GATACCGTCGCCATTCAGGCTG |

TABLE 4
Staple sequences used for the T1 ring triangle folding

| SEQ | |
|---|---|
| ID NO: | |
| | Core structure oligo sequences (5′-3′) |
| 416 | GCAAAGACAAGGTGGCAACATATATGGTGATGGTGGTTCCGAATAGCC |
| 417 | TTCCATTATAAATTGGGTCAGGACACAGGTAGAGGTCTTTA |
| 418 | ATGCGCCGCTACAGGGAACGTGCTGGAGGCCG |
| 419 | TCTCACGGTACATCGACATGGAGAGGGTAGCT |
| 420 | GGGATAGCGGAACGCCTCTGGAGCAAACAAGAAAAGGCCG |
| 421 | AGAGAGAAATTGAGTTAAGCCCAAGAGATAAC |
| 422 | TTTTTCAAAATTTAAGACGCTAATTTTC |
| 423 | CCTGACTATTATAGTCAGCTTCAATGAATTACCAACAGTT |
| 424 | CGGAACAAGATTTACACCAGAAACAAAGAAAACGAAAGCGCG |
| 425 | CGGTGCCCTCGTTAACGGCATCACCACGGGACAGCGGTTTGTTA |
| 426 | GCGGTGCTGTCACTCGGGCGCCCAGCATCCGCCAG |
| 427 | AACAACATGTTCAGCTCGAGCATGTTTTTAACCAAATATCCTAAAGCA |
| 428 | TCTGACCTAATATATTTGCAAATCCAATCGC |
| 429 | GAAATACAAATGCTTTAAAAGATTAAGAGGCGCGAGAAAAC |
| 430 | ATCAGTTGACATTATTGTTGGGAAGAAAAATGGGAGTT |
| 431 | GGAATACCACATTTACGAGCCGGAACCTGTCGTGCCAAAACGAACTAA |
| 432 | GCGCGCCTGTGCAAATAAGAGAATAACAATAGATAAGACCTGCAGCCA |
| 433 | AAATAAGGCGTTATATATTAATTGAGAAGAGAACCTACCACAAAGAA |
| 434 | GCGGAATCGAGAATGACCATAAATCAATTTTTAATCAAAGATTC |
| 435 | CAATACTTTGATAAGAGCGAACCATTTTCTGTCAGCGGACGAATAAT |
| 436 | TTACGGGAATCAACGTACGAGTAGAACGGGTAAAAGGCCG |
| 437 | TGAAATTGGCCTGGGGTGCCTAATGAGTTTTTGAGCAGACGATCCAGCGCAG |
| 438 | TCCTGTGCCGCCTGGGTATTGGGGGGACGACGCCAGCTT |
| 439 | CATGTAATAAGGTAAAGTAATTCTGTCTTTTTAGACTTTCATCT |
| 440 | ACGCCAACCGCACTCTAGAAACCTGAAAAATAAACCCTC |
| 441 | AGAATAACTTTTTTCAAGAAAGCTTTGATTGCTATTTATTTA |
| 442 | ACGTTATACAACTAAGAACCCATAGACGTTAGCCCTCATCCTTTAAT |
| 443 | ACAAACAATTCGACAACTCGTATCTGGCCAAAATTATTTGCACGAAG |
| 444 | ATACATTTGAGGATTTAGAAGTATGAAAGCGT |
| 445 | TTAATGCGCGAACTGACTAAAATAAATTCATCCTGAACCT |
| 446 | GGCGAAAGGGGGATGTGCTGCAAGAACCAATA |
| 447 | GTCTGGCCTTTTTTTCTGTAGCCAAAGGTTTTTTATCAGGTCATTG |
| 448 | GCTTTCATCAACATTAGCGCAACTGAATTTGTGGAAGATC |
| 449 | GTTGGGTAACGCCAGGGTTTTCCTGATAATCATCAAACTTAAATCTG |
| 450 | ACCGCCACCCTTTTTCAGAACCGCACGGTTTTTGTCAGTGCCTTGA |
| 451 | CACCCTCAGAGCCACCAAATCTCCAGCAACGGACAACTTT |
| 452 | ATAGCAAGCCCAATAGAGGAATTGGTGAGAATAGAAAGGA |
| 453 | TTTTGAATGGTTTTTTATTAGTCTAGTATTTTTAAGAACTCAAACT |
| 454 | AAAACGACGTTGGCAAATCAACAGAATCAATATCTGGTCA |
| 455 | CTTGCTTTCGAGGTGAATTTCTTATACTCAGG |
| 456 | AATTTTTTCACGTTGAACCCTCATTTTGCTAAATGATACAAACGCCTG |
| 457 | CCTGAGAGATCAAAAATAATTCGCCGCCAGCTCGCCATGTTT |
| 458 | TACGGTGTATTTTATCCATTACCAGGCGCTAGGGCGCTGCGCGCTTA |
| 459 | GCAAAATCTTAGCTATATTTATAACCACGG |
| 460 | CGAGATATCCACTATTATTTTCGTCTCTTTTTTCGCGCAA |
| 461 | AAAACGCTCTATAAAACAGAAATACCTTAGAATGAATTAC |
| 462 | TACCGCCAAGAACCCTTCTGACCTTAGACTTTACCACCAGAAGGAGCG |
| 463 | ATCGGCCTGTGGCACAGACAATATAATAGATACCTGATTATC |
| 464 | GCGCATTAGATCGCGCAGAGGCGTAGATACCAAG |
| 465 | TTTGCACCCAGCTACAGAGGTTTTAATTACATAAAAATTACTAGAAAA |
| 466 | CATCAATATTTTTGATATTCAACCGTTCTCGTGAGAGATCTACA |
| 467 | TAAAGGTGAAAATCCGCGACCTGCATTGATAAATCCGCCTCC |
| 468 | CCAAAAGAACAACGCGGTCCGTTAAGGATTGCCGTGTACCA |
| 469 | GTCAAAGGCAGTTTGGGTAGAACGGTAGGGGGTTTCTGCC |
| 470 | GACGATTGTTTTTCCTTGATATTCACAACTGGTAATAAGTTTTA |
| 471 | AGTTGATACCATTAGATACTCCATGTTATTTTTTTAGTTGACGGA |
| 472 | GTAACAGTTACCGCCACCCTCAGATTTATCAGGACAGCATCG |
| 473 | AATCAGTGTTTTTGGCCACCGAGTAAAAAAACATCACTTGCCTG |
| 474 | TTAACGTCTTTTTAAAATGAAGGGAGCCCCCTTTTTATTTAGAGCTTGGTTTTTAT |
| 475 | CTCAGAGCATAAAGCTTAAGAAAAGTAAGCAGATAGCCG |
| 476 | TAATTTGCCAGTTACAAAATAAAAAGGAGCGTTAGATTCGCCTGATT |
| 477 | GTCATAGCTGTGTCGACCCCAGCGTGGCTGACTTACCCAATAGCGTC |
| 478 | TTCATCGGCATTTTCGAGCGCCAAAGACAAAAGGGCGAC |
| 479 | GAGAAAGGACAGGAACGGTACGCAACAATATTTTTCAGG |
| 480 | GTAATTGAAAGTTTTTCAAGCAAGACCAAGTA |
| 481 | ACAAATAACTCTGAATTTATTTTTCGTTCCAGTTCAAGGTTGAG |
| 482 | ATCCTATCAGGGCGATGGCGGGTAAAGTTAAACCGGACTTAACAAGAGGGGTTGAG |
| 483 | TTCCGGCAGTAAAAAAAATGCCAATTACGGCTAGCTGTT |
| 484 | TACTCATTTGGGGCGCGACGGAGATTTGTATTGACCAACTTAGTTTGTCCCAATT |
| 485 | TTATCACCGTCACCGTTTTTCTTGAGCCATTTGGAATTATTCAT |
| 486 | GAATTAGAAGTAGCGATAAAGCCATAGCAGCAAGTTTCGTCACAGACAGTAAATGA |
| 487 | AATCACCAGTAGCTCAACATGGAACGAGGCGCAG |
| 488 | GGTAAATAGCGTCTTTCCGTAATCGCCAGCAAACCATCGATTTGCGGATGCTCCTT |
| 489 | AAAATCCCTGGCATGATTAAGACTTTTTCCTTATTACGCAGTAACGGAATAC |
| 490 | ATTCTACTAAAATACACGAAAATCCTGTTCAGCCTCCGGCCA |
| 491 | TACAAGCTGATGAACGTCAGTGAAACACAACTAATCGCCAAAAGGAA |
| 492 | AGCGCAGTATCCTCATCAGAATCAAGTTTGCCCGATTGAGGGAGGGAA |
| 493 | ATAACGGCAATTTCATTTCCTTGAACCTCCGACCGTGTGAT |
| 494 | GTAACAGTCAGTACATATCGTCGCTTTAACA |
| 495 | AATATACACGGGAGAATTAACTGTTTTTACACCCTGAACAAAAAACAGGGAA |
| 496 | AGCAATACATGTGAGAAGTACGGGGAAAGCCGGCGAAACGATTTTTTGT |
| 497 | AATAAATAAATATAATCCTGTTTTTTTGTTTGGATTATATCATATT |
| 498 | ATTAACACCGCTTCTGCTCATTTGCAGCGGGGGTCTGGTC |
| 499 | ATTTTGATGAGAGATAGACTTTTTTTCTCCGTGGTGAAACGTACAG |
| 500 | GAACAAAACATCCAATAAATCTGATATTTTTATTAATGCCAAAGAGACAGT |
| 501 | ATTCAACTTTAGCGTCAGTTTTTCTGTAGCGCGTTGCAGGTCA |
| 502 | GGCTTTTGCTACAGAGGCTTTTTTTGAGGACTAAAGACCAGCGAAA |
| 503 | GTAACGATCCAGTCACACGACCGTAACACTGAGCGGTCCCAATGAA |
| 504 | GCGCTAATGCGCCCAATAGCACCTGAGCAAAAGA |
| 505 | TCACCTTGCCGAACGAACCACCAGAGGACGCAAATTAACCGTTG |
| 506 | CCACGCTGAGAGCCAGGTGAGGCGGTATTAACCGTTTTTGTAGGGCT |
| 507 | ACACCGGAATCATAGAAGTTTTGCTGAATCCC |
| 508 | ATGGGTAAGTGGTGCCATCTTTTTCACGCAACCAGCCGGCAGC |
| 509 | AACCGTGCATCTGCCATGGGATAAGCTGATTGCAAGCGG |
| 510 | CACTACGGGGTTGCCCTGATAGCTGCATTAATGAATCGGCCTAACCGA |
| 511 | AACAAAGATTAGCAAAATTTTTTAAGCAATAAAGCCAAATCAC |
| 512 | AGATAAGGCACCAACCTCTGCTCATGTGTACAGAGCAACACTATCATGAGG |
| 513 | GAGGAAGTCACTAAAACACAAGCGTCATACAT |
| 514 | TTTTTCATAGGTTTAGGCCCGTATAAACAGTTTTGACAGGTCTTTGAC |
| 515 | TATGGGATTTTCAGGGCAAACTACGGAGTGTA |
| 516 | CCACGCAGTGCCGGAAACCAGGCTCCGGCACCGCTTCTG |
| 517 | GATACCGATAGTTGCGCCGACAAAGGCTGAGTAATGC |
| 518 | ATAATGGATTTAACGTCAGTTCTTTGATTAGT |
| 519 | ACTTCTGAAAGAATACTGCTGGTAATATCCAGCAGAATCC |
| 520 | AGATGATGGCTCTTTAGGAGCACTAACAACTATTTTTTAGATTAGAGCCGTC |
| 521 | ACCAGTCCCGGTTGGGAAGGGCGATCGGTGCGTTTTTGCCTCTTCGCTATTA |
| 522 | GAACGAGGGTAAAAAAAAGGCTCCAAAAGGAGTTTTTCTTTAATTGTATCGG |
| 523 | GAGTAACATTATCATTAAGACAAATTAGATTAATGGTTT |
| 524 | AGGGTTAGTCAATAGTAATGCTGATTAGTTAAGACGACAAT |
| 525 | CTCCGGCTTAGGTTTTTTGGGTTATATAACGAATTATC |
| 526 | ATCCCATCATCGGCTGACCGACAATTAGGCAGAGGCATT |
| 527 | TCAGCAAACCTGCATCTAACTCACATTTTTTAATTGCGTTGCGCTC |
| 528 | TGGTGTGTGGGTCACTGTTGCCCTGTTTTTGGCTGGTA |
| 529 | TTGCTCGTCATATTTTTACATCCCTTACACTCGGCGAA |
| 530 | CGGTTTGCAGGTTTCTGCACTCCAGACAGTATCGGCCTCATCTCCGTGG |
| 531 | AACGCGCGTGGTTTTTAGTGTAAATTATCCGCTCACAAT |
| 532 | TTATACCAGCTTGAGATGGTTTAATTTTTTTCAACTTT |
| 533 | CTTATGCGATTTTTTTTAAGAACTGGCTCAACCCTCAG |
| 534 | ATCGCGTTAGCAAACTCAGAAAACGTCATAAATATTCAT |
| 535 | TATATGTAGAATTTATCAAAATCATTTTTTGGTCTGAG |
| 536 | AGACTACCGAGTGAATAACTTTTTTTGCTTCTGTAAAAATCAA |
| 537 | AATCAATACTAATTTAAATGCAGAACGCGCCTGTTTATCATAAAGT |
| 538 | TCTTTCCTTATCGCTCAACAGTGGCCAAGCTACGTTGT |
| 539 | TTCGCACTGTTTCCTGAACAAGAAAAATAATGGCCAGT |
| 540 | TTTGAAAGAGTTAACCCTCGTTTACCAGACGACGAT |
| 541 | CGCCAGGGGGGAGAGGACTGCCCGCTTTCCAGTCGGGAAAGCATAA |
| 542 | CTTTTCACCAGTGGCCGCATCGTTATATTCGACCATCGC |
| 543 | AATCATTGATCTTGACAAGTTTTTACCGGATATTCACTTCATC |
| 544 | AGACCGGATTAATTCGAGAAGCAAAGCGGATTGCATCAAAACAGTT |
| 545 | CCAACAGGTCAGATATTTTGTCGAAAAGTTTTTGCCCGA |
| 546 | GCGATAGCGAAAAGCCCGAAAGACTTCAAATTAATTTT |
| 547 | GGCATTCCAAGAACGGGTCAGTACCATCACCCAAATC |
| 548 | TCATTGCAAGTCTCTGTGGTGCTGCGGCCAGAATGCCAT |
| 549 | GGAGGTGTGGTTGCGGACGCAGAAAACGGATA |
| 550 | AACGATTAGAGAGTAAGTTAGCAACGTCAGAGCGGGAG |
| 551 | TATATGTATCGAGAATGGGGTCGCAGAAGATAAAACAGAGCAGCAAA |
| 552 | ACCGTCGCCCTGAGATAGCATTACGGCGGATTGACCGTAAGTTTGAG |
| 553 | AAGAGTAAGGTCATTGAATGGAATAGCATTCCACCAGTA |
| 554 | TGGCTTAGGGTAATAGCCAAAATAGCGAGAGTTCATTCACTAAAG |
| 555 | GAGCACATCCTCATAACGACCGCAAGAGCCGCACCAGTTGGG |
| 556 | GCTTTTGCTTAGAATATAATGCTGTAGCACCTGAATC |
| 557 | ACCGAACCATCGCCTTCGCAAATAGAAAATTCATATGGTTTACC |
| 558 | ACGGTCAATCATAAGGGAGCATAGGCATTATACCAAGAGGCA |
| 559 | ACAAAATTGAAGCCTTACCTCCCGACTTGCGGCTGGAAGT |
| 560 | AGATGATGAAACAAACATAATGGAAAACCTTTTATGCGTAGA |
| 561 | ATGCGTTATACAAATAGAACGCTAGAAGGCTTATCCG |
| 562 | AGCACGCGTGCCTTTTTGTTCATTCGTAATTTTTTATGGTGGCGG |
| 563 | AAAATAAAATGTTTTTTTAGACTAGGCATAGTAAGACCAGGC |
| 564 | GCCGTTTAGCAGCAGAACGTGCGATGCTAACGTGGAGCTATC |
| 565 | AGCCTGTTTAGTTTTTTTCATTAATTGAGATTTTTTCGCCAAATA |
| 566 | CTCAACAATTTTCATCGTAGCGCTACGTGA |
| 567 | TCACGGTCATACCTAAAGCCAACGTTCGAGCC |
| 568 | GGGTACCTCCACGCTGGTTACAGAAAAGGTTTGGTGT |
| 569 | GAGCTCGATTCGCGTCCGTGAGCATCAAAAGAAATCG |
| 570 | ATCCCCTCCACACATAAGAGACGGGCAACGGTCACGGGCATCA |
| 571 | CAGAGGGAGCTTAATTGCTACAAGGCCGGA |
| 572 | TTTTTAAACAGGAAGATTAACTAGCATGTCAATCATATTTTT |
| 573 | TTTTTCAATGCCTGAGTAATGTGTCTTTAGTGATGAACCATGGTGCTG |
| 574 | TTTTTAGCACTAAATCGGAACCCTAAAAATAGCAGCCTTTAC |
| 575 | TTTTTCATCGCCATTAGAGTCTGTCCATCTGCCGTAATTTTT |
| 576 | TTGAAAGGAATTGAGGAAGGTTATTAGCCCTAAAATTTTT |
| 577 | TTTTTTGTACCCCGGTCAGTCACGTTCAGAGGTGGAGCCGGATGCCGGCAATCCGC |
| 578 | GCGAAAAACCGTAGATAATAAGAGCAAGAAACAATGAATATTTTAAATGTTTTT |
| 579 | TTTTTTAAAGGGTGAGGAATCGATCGGTTGTGAAAAAGAGTATGAGCC |
| 580 | GATTCAAATACTTTTGAAATATTTAAATTGTAAACGTTTTTT |
| 581 | GAACGGTAAATCAGCTAAATTCGCATTAAATTAGAAAAGCCCCAATTTTT |
| 582 | TTTTTTAATATTTTGTTACATTTTTTGCGATTAAACCTCACCGGAAACAA |
| 583 | TTTTTCGCAAGGATAAAACCCTCATAATAGCAATACTCCAAC |
| 584 | ATCGTAAGTATAAGCCGGGAGAAGCCTTTATTTCAATTTTT |
| 585 | AGGCGGCAGGTAAAAAATAGAAATTTTACATTATGACCCTGTTTTT |
| 586 | TTTTTGCCACCACCCTCAGAGCCGCCTCTGAAACATGTTTTT |
| 587 | AAAGCGCCATTCGCCATTCAGGCTAATGTGAGCGATTTTT |
| 588 | TTTTTGTAACAACCCGTAGCATACAGGTTTTT |
| 589 | TTTTTCAAGGCAAAGATTACCAGAAGGAAACCGAGGAAACAAAGAAAC |
| 590 | TTTTTAAAGTATTAAGTGACAACAGTCGCTGAGGCTTGCACTACGTTAACGAGAAA |
| 591 | CACCACCCTCAGAGAGAGCCACCACCGGAACCCCTTATTAGCGTTTTT |
| 592 | AGAGCCGCCATAATCATTGCTCAGTACCAGGCGGATATTTTT |
| 593 | TTTTTAGGATTAGGATTACCTATTATACCAGAACAAACAAAG |
| 594 | TTTTTACCCCGCCACCCACAATCAATGGTCAATAACCTGTGAGTAGAT |
| 595 | CTGCCTATAATAGGTGGGGTTGATATAAGTATACTCCTCAAGAGATTTTT |
| 596 | AAATACGAGCCCGGTTCGGAAGCGGGGTTAAATCACCGGATTTTT |
| 597 | TTTTTTTTGCCATCTTTTCGCCAGCAAATGCCCCAAAGAATA |
| 598 | TTTTTAGTGCCGTCGAGATATCACCGAACAGCTTCTTTTGCGGGATCGTC |
| 599 | TTGCGGAATATCAACAGAGATGCCATTGTTTTGT |
| 600 | AAGAAATCATCGGGGATTTTAGAAGGGAAATGGTT |
| 601 | CTAAACATTCCTCGTTAGAATCAACGCTGCGCGT |
| 602 | TCTTTCCGTACCAGTAATAAAATACATTGG |
| 603 | CTCAATCGTCTGAAATTACCTACACAACAGGAATTAAAGGAGAAACA |
| 604 | TATTCACATGGAAAGGATTATTGGGACATTTAAATCCT |
| 605 | TCCACACCCGCGCAAGTGTCCAGAGCCTTACCAAC |
| 606 | TGACGAGCACGTATCGCGTACTGAAAGCGACAGCCATA |
| | H1 design oligo sequences (5′-3′) |
| 607 | GCAGTAGAGTAGGTAGAGATTAGGCACCACAAGATTAAGCAAATCAGATAGAGGCGTTTTTTTTAGCG |
| | AAAATCAA |
| 608 | GCAGTAGAGTAGGTAGAGATTAGGCATAATATGTTAGCAAACGTAGAATAGTAGGAGTTGCAGCCCTT |
| | CA |
| 609 | GCAGTAGAGTAGGTAGAGATTAGGCAAATAAGTTTATTTTGTCTCAGAACCGCCACCCTCAGATTTTT |
| 610 | GCAGTAGAGTAGGTAGAGATTAGGCATCCCAATCCAAATAAGAACGTGGCTTACAAAA |
| 611 | GCAGTAGAGTAGGTAGAGATTAGGCACTGCGAACCCTTATAACTCCTCACAGGTGCCCCAGCAGG |
| 612 | GCAGTAGAGTAGGTAGAGATTAGGCATTACCGAAGCCCTTTTAAATCGGT |
| 613 | GCAGTAGAGTAGGTAGAGATTAGGCAGCTAACGACCGTTTTAAATATGCACATATAAC |
| 614 | GCAGTAGAGTAGGTAGAGATTAGGCAGATAATTATTCATTTTTTTCAATAACATAAGTCAGAGG |
| 615 | GCAGTAGAGTAGGTAGAGATTAGGCATGTTGTTCGTATTCTATCTTACCATCAGGAATCATTACC |
| | H2 design oligo sequences (5′-3′) |
| 616 | GCAGTAGAGTAGGTAGAGATTAGGCATTTTTTTTTTTTTTTTTTTTCCACAAGATTAAGCAAATCAGATA |
| | GAGGCGTTTTTTTTAGCGAAAATCAA |
| 617 | GCAGTAGAGTAGGTAGAGATTAGGCATTTTTTTTTTTTTTTTTTTTAATATGTTAGCAAACGTAGAATA |
| | GTAGGAGTTGCAGCCCTTCA |
| 618 | GCAGTAGAGTAGGTAGAGATTAGGCATTTTTTTTTTTTTTTTTTTTAATAAGTTTATTTTGTCTCAGAACC |
| | GCCACCCTCAGATTTTT |
| 619 | GCAGTAGAGTAGGTAGAGATTAGGCATTTTTTTTTTTTTTTTTTTTCCCAATCCAAATAAGAACGTGGC |
| | TTACAAAA |
| 620 | GCAGTAGAGTAGGTAGAGATTAGGCATTTTTTTTTTTTTTTTTTTTCTGCGAACCETTATAACTCCTCAC |
| | AGGTGCCCCAGCAGG |
| 621 | GCAGTAGAGTAGGTAGAGATTAGGCATTTTTTTTTTTTTTTTTTTTACCGAAGCCCTTTTAAATCGGT |
| 622 | GCAGTAGAGTAGGTAGAGATTAGGCATTTTTTTTTTTTTTTTTTTTGCTAACGACCGTTTTAAATATGCA |
| | CATATAAC |
| 623 | GCAGTAGAGTAGGTAGAGATTAGGCATTTTTTTTTTTTTTTTTTTTGATAATTATTCATTTTTTTCAATAA |
| | CATAAGTCAGAGG |
| 624 | GCAGTAGAGTAGGTAGAGATTAGGCATTTTTTTTTTTTTTTTTTTTGTTGTTCGTATTCTATCTTACCAT |
| | CAGGAATCATTACC |
| | H3 design oligo sequences (5′-3′) |
| 625 | GCAGTAGAGTAGGTAGAGATTAGGCATTGCAGTAGAGTAGGTAGAGATTAGGCACCACAAGATTAAG |
| | CAAATCAGATAGAGGCGTTTTTTTTAGCGAAAATCAA |
| 626 | GCAGTAGAGTAGGTAGAGATTAGGCATTGCAGTAGAGTAGGTAGAGATTAGGCATAATATGTTAGCAA |
| | ACGTAGAATAGTAGGAGTTGCAGCCCTTCA |
| 627 | GCAGTAGAGTAGGTAGAGATTAGGCATTGCAGTAGAGTAGGTAGAGATTAGGCATAATAAGTTTATTT |
| | TGTCTCAGAACCGCCACCCTCAGATTTTT |
| 628 | GCAGTAGAGTAGGTAGAGATTAGGCATTGCAGTAGAGTAGGTAGAGATTAGGCATCCCAATCCAAAT |
| | AAGAACGTGGCTTACAAAA |
| 629 | GCAGTAGAGTAGGTAGAGATTAGGCATTGCAGTAGAGTAGGTAGAGATTAGGCACTGCGAACCCTTA |
| | TAACTCCTCACAGGTGCCCCAGCAGG |
| 630 | GCAGTAGAGTAGGTAGAGATTAGGCATTGCAGTAGAGTAGGTAGAGATTAGGCATTACCGAAGCCCT |
| | TTTAAATCGGT |
| 631 | GCAGTAGAGTAGGTAGAGATTAGGCATTGCAGTAGAGTAGGTAGAGATTAGGCAGCTAACGACCGTT |
| | TTAAATATGCACATATAAC |
| 632 | GCAGTAGAGTAGGTAGAGATTAGGCATTGCAGTAGAGTAGGTAGAGATTAGGCAGATAATTATTCATT |
| | TTTTTCAATAACATAAGTCAGAGG |
| 633 | GCAGTAGAGTAGGTAGAGATTAGGCATTGCAGTAGAGTAGGTAGAGATTAGGCATGTTGTTCGTATTC |
| | TATCTTACCATCAGGAATCATTACC |

TABLE 5
Staple sequences used for the T3 triangle 1 folding.

SEQ ID NO:

Core structure oligo sequences (5′-3′)

| | |
|---|---|
| 634 | TATCAGGGACGGGGAAAGCACTAAAGCGGGAGTAGACAGG |
| 635 | AGAACGGGATCCGGTAAGCAGCCTTAACGTCAGTGAGAGA |
| 636 | AAAGTGTCTGCCCGCTTTCCAGCGGTGCCGGGGGTTTCTGCCAGC |
| 637 | TATTAGCTGGTAATATAATACCTATCCTCGTTCAGGGCGC |
| 638 | GGAAACAATCGGCGAAGATAGCTCAAGAAACACGGAATTT |
| 639 | AACAAAATTAATTACATAAGTATACCACCCTCAGACTCCT |
| 640 | GCAGAGGCATAACGGATGAGTTTCTCCACAGACCTTGAGCAAATAA |
| 641 | CCACCCTCTAGGTGTATCACCGTAAAACAAACTTACCTGA |
| 642 | TAACAGTGCCCGTATAGGTCAGTGCAGCCCTCATAGGAGCGTAGATT |
| 643 | CATACTGGTAATAAGTTTTAACGGAACAGTTACATGTACGGATAGC |
| 644 | TTCTAAGAGAGAAACAGAATTATTCAGAGCCAGCCCGGAA |
| 645 | AATAATATCGTTTTAGCGAACCTCACGATTTTTCTTACCAACG |
| 646 | ATCGTCGCGATGCAAATCCTTTTTATCGCAAGACAAAGATGATG |
| 647 | AGCGGTGCTCGGGAAAGATCCCCGACACAACAGCAGCAAGCGG |
| 648 | AATAAGAACCGACTTGCGGGAGGTAATAGATAAGTCCTGA |
| 649 | CAAGTACCGCACTCATTAGGAATCCTCACATTGTGTCACT |
| 650 | TTCGCCTGTTCAGGTTTTACAGAGCGGCAATAATAAGAGCTCACGGAATTCTCCGT |
| 651 | GTAGAAGAACTCAAATCTTTGATCAAATTAACCGTTGTA |
| 652 | GAATGCGGCGGGCCGAGGCGGATGTGTTTTTCTTTTCTGAAACATGA |
| 653 | CCGCGCTTAATGCGCCGCTAAGAATCAGATCGGAACATCCGCTC |
| 654 | CCAGAACAAACGGTACTGAGGCCAGCTCGAATGTCATACCCCCTGCAT |
| 655 | CCAAGCTTTCAGAGGTGGAGCCGCCATTTTTGGGAACGGATA |
| 656 | CAAGTTACTGCGGCCAGCGCGCCTGTGCACTCTTTAACAAAGCATGTA |
| 657 | GCAATAGCTATCTTACACCTCACCCGGAAATTTTTTTATTCATTAAA |
| 658 | AAGTATTAATTAGCGGTCGAGAGGGTTGATATGTGGTGC |
| 659 | CAGACGATCCAGCGCAAATTGCGTATGGTCATAGCTTACAAGCCCAA |
| 660 | GTACATAAATCAATATATGTGAGTGATTTTTTAACCTTG |
| 661 | AATGAGTGGAAGTTTCCTGTGTGCCTGAGAAACGTGCTT |
| 662 | CTTTCAACAATTTACCGTTTTTTTCAGTAAGCGTCATACATGG |
| 663 | AAGTGCCGGGTTTTGCTCAGTACCGCAATACTCTATCGGCATAATCAG |
| 664 | CAGTTGAGCCTGTCGTAGAGAGTTTACGAGCCCCGTGGTGGTTCCGAA |
| 665 | AAAATCGCGCAAGCAAATCAGTAAATTACCGCGCCCAATAGAAACCAA |
| 666 | GATGAATATGTAGCATGTCACCAGTACAAACTGCCACCCTCATTTCAA |
| 667 | AGAGCCGCAAGAGAAGGATTAGGAGAGGCTGATTTTCAGCGTAACAC |
| 668 | AAGGTAAATATTGTTTACCAGTCCATGAAATACGTCAAAAATGAAAAT |
| 669 | GAGATAAAGAAATTGCACGTATATAGGAACCTCGGAACC |
| 670 | TTTAGCACAACGCCTACAGTAACAGTAGAATTGTTTAA |
| 671 | ATTCACAAACCACCAGAACCATTTTTCACCAGAGCCGC |
| 672 | TTTGATTTTTAGCCTTAAATCATTATTTATTTTTCCCAATCCA |
| 673 | CCGATTTACTTCTGAATTTCTGCTCATTTGCCGCCAGC |
| 674 | TAACGAGCGTTAGATTTTGCACCTTTTTAGCTACAGAACGCGA |
| 675 | CGAAGCCCTTTTTATTTTTGAAAAGTAAGCAGATAGCCGA |
| 676 | GTAACGATAGCAAAATTATTTGCGCATTAGAAGAATAACGATAACCC |
| 677 | AGCGGATCAGGGAAGCACGTAAAACAGAACCCGTACTATGTAATCCTGTCAGATGA |
| 678 | ATCAAGAAGAATTACCTTTTTTAATGGAAACAACAAGAAAGCAAAAGA |
| 679 | CCCTCAGAACCGTCAGACTTTTTTTAGCGCG |
| 680 | GATACAGGAGTGTTTTGACGCTCACCTGATTAATTGTTTGGATCTGGCA |
| 681 | ATAATGGAAGGGTTACATCAATAGTTGCTTT |
| 682 | GGGATTTTGTTAGTAAATGAATTGAACCTACCATATACTAAAGAGACGCAGAAAC |
| 683 | TTTGGGGTCGAGGTGGGAAGCATACAATTCC |
| 684 | CTTCTGTAACAGAACGCGCCTGTTTTTTTATCAAC |
| 685 | AGGGCGAAAAACCGTCAGTTGGGCAAAGGAGC |
| 686 | GTCACCTTTTTACTTGAGACGTACAGCGCCATGA |
| 687 | ATCCCGTAGAAGATTTTTAAGCGGGTTGTGTACATCGAC |
| 688 | TTAGAAGTGCCATTGGTGAGGCGGTGCCTG |
| 689 | AGGAATACCGAACGAACCACCAGCCCACGCTGCACCTTGCTGAACCT |
| 690 | TCATATTATCGTCTGATTTTTATGGATTACAACAGGAAATTTTTACGCTCATGGA |
| 691 | AGAACCCTTTACATTGGCAGATACCAGAAGATCATTTTGCGGAACAATGCTGAT |
| 692 | CATCGCCATTAAACAAACTGATAGTGGCTATTAGTCTTTA |
| 693 | CAGCAGGCTTTTTAAAATCCTGTTTGATAAAGCCGGCGAACGTGG |
| 694 | TGGTCAGCAGCAACCGAGCACATCAATTTTAAAAGTTAACCACCACACCC |
| 695 | ACGTTATTCTCATAACTGCCGTTCAGGGTAAAGTTAAACGAGTTTGAG |
| 696 | AATCATACTGACCATTTTCTGCGAGCTTGCCC |
| 697 | GTTCAGCAAATCGTTAACTTTTTGCATCAGATGCCGGGTTACCTGCAGCC |
| 698 | GGTACCGACCGAGTAAAAGAGCAGTTGTTTTTAAGGAATTGA |
| 699 | TCCTCACTGTTCTTTTTTTGCGTCCGTGAGCCGGGTCACTGT |
| 700 | CCAACGGCAGCACCCCAGCCCGAGGAGTCCACTATTAAAG |
| 701 | GCTCATGCAAATAGATTTAAAGCATAGAGCCAGCAGCAAATGGGCGCGA |
| 702 | CTTATAAATCGCTGATTGCCCTTTTTTTACCGCCTGGCCCTG |
| 703 | TTTTAATGACAGTATCGGCCTCAGGAAGATAATATTC |
| 704 | CTAACGGAACAACATTATTACATTTCAACTTTAATCCAGCGATT |
| 705 | GCCAGCTGCATTAATGATTTTTTCGGCCAACGCGCGG |
| 706 | ATATTATTTTTCGCCAATTAGACTGGAAGGTTAATCCGCCTGCCCTGC |
| 707 | CCGATTATTTTTAGGGATTTCTAAACTTTTTGGAGGTTACAAAC |
| 708 | GAACTGACCAACTTTGCCGAATAACCTGTTTAGGTGGCATC |
| 709 | AGACAATATCTGGCCATAAGAATACAATAGAAACAACT |
| 710 | GCTATATTTTCATTTGCAATCATAAACGTAACAAAGCT |
| 711 | AGGTCTTTCAAAAGGATGTTTTAAAACAGTTGATTCCCAA |
| 712 | AAAAAAGCCGCACAGGCGGCCTTTAGTGATGACGGCAAAC |
| 713 | TGTTACTTGTCATTGCAGGCGCTTCAACCAGCTTGAGGATAACTCGTA |
| 714 | CTTTTGATGCTCAACACAGTTCAGTGACGAGAAACACCAGATTCATTA |
| 715 | AATCAATATCTGGTCAGTTGTTTTTCAAATACGCGTGC |
| 716 | AAAACGAGAATACGTCAGCGTGGTTAATTGCAGAGCCGT |
| 717 | AGATACATCCCAAATCAGGGAACCTTTGTATCATCGCCTGATAAATTG |
| 718 | TCAGCGGGAGCCGGAATCCGCGACCTGCTCCAAGGTTTCT |
| 719 | GAGGAAAGCCAGCTTTCCGCAATACACTATCATAACCCTCGT |
| 720 | GGAACGTGCCGGACTTGTAGAGACATTTTTGCGCGCATCGGATAGGTC |
| 721 | ATGCGCGAGGCAAAGAGTACCAAAATGCTGTAAAGAGGTCCATAAAT |
| 722 | TTGCTCGTGGCTGGTAATGGGTAACAAATATCTATCTTTA |
| 723 | TGTCGAAACGAGGCGCAGACGGTGAAAAATCATTTAGTT |
| 724 | AGTGTAGCGTTTTTTCACGCTGCTTTTTTCGTCTCGAGG |
| 725 | GGGACGACTAACCGTGCATCTGCCGTAGATGGGGATGGCTCACACGAC |
| 726 | CAGATGAAATACCAAGCGCGAAACAAAGTACAACGGAGA |
| 727 | ACCGCCTGCAACAGTGAGAAGATAGGAGCACTTAATACATTTACGGCT |
| 728 | TAAAAAAAACGTGGACTTTTTCCAACGTCAAATCGGCAAAATC |
| 729 | CAATCTGTCCATCACGTAGTAATAACTTTTTTCACTTCAG |
| 730 | AAACAGAGTTCTGACCTGAAAGCGACAGAGATTTAAATCCTTTGAGC |
| 731 | AAGAGCAACTGCGGAATCGTCATACGCACTCCGCCCGAAA |
| 732 | GGGCGCGGGGTTTGCGAGTGAGACGGGCAACAAAAAGAAT |
| 733 | TGCTGGTCGGAGGTGTCCATCAGTGAATAAGACGAGTAGTGAATATA |
| 734 | CATAAACATCCCTTACACTGGTGTGGAGAGGCTTGCGGTATGAGCC |
| 735 | AACATAAAAGGGACATTTTTTGAACCCTAAAACGTGGCAC |
| 736 | ACCCTGACTATTATAGTTTTTTAGAAGCCCACATTCATTTTTCTAA |
| 737 | TCAAAAGGATTCGCAGTAAACGCTCAGCAGAATCATAGAAGAGTCA |
| 738 | ATATGCTTTTTACTAAAGTACGGAGAGTACTTTTTTTTAATTGCTC |
| 739 | CTCCGGCTAACATAGCGATAGCTTAGATTAAGTTAATTGATTGAAAT |
| 740 | ATTCGCGTCTGGCAAACAGCTTGGCGGGATCGTCCCGTGTGATAAA |
| 741 | GGGCGATCGGTGCGGCGACAATGAACGCCATTCAGCTCAAAGCCTC |
| 742 | TCTTTGACCCCATTGTGAATTTTTTACCTTATGCGATAATAAAACGA |
| 743 | ACCGAACCTTAATATTTTGCCTGAAGATCTACAAAGGCTACGCCACCC |
| 744 | TCCTCATTAAAGCCCAGACGATCGGTCATAATCAAAATACCTAAAT |
| 745 | TTCTAGCTGAAGTAGTAGTTTTTATTAACATCCAATAGCTGAAAA |
| 746 | GTGAGAAAGGCCGTTGAGGCAGGTAGAATGGAAGCACCGTTACCATTA |
| 747 | ATAATTACCTTTCCAGAGCCTTTTGACATTCAACCGATTGAGGGAGG |
| 748 | CAAAATAAACAGCCATAAGATTAGTTGCTAAAACATGTTCAGCTAATG |
| 749 | ACGACGACTTAATTTCCCGGAATCTTTTCATCATCAAGTTTGCC |
| 750 | CGCCAGCATCAGAACCCCGCCTCCCTCAGAGCTTTTTAACTTAATGGT |
| 751 | AATCGGTTATTAGCAAAATTTTTTAAGCAATATTTTTTAAACAGGAAG |
| 752 | AAGAGAATAGCAAATATTCAACCGATGTGTAGGTAAAGATTGCAATGC |
| 753 | CAGTAATTATGACCCTGTAATTTTTTCTTTTGCGGGAGAGCATAAAGCT |
| 754 | GAAACCGAAAAAGGGCTGTCACAAGCGACAGAGGCATTTTTGGCCTTGA |
| 755 | AAAACTTTTTCAAATATAAATGCTTATTAATTAATTTTCCCTTAGAAT |
| 756 | GCTACAGAGGCTTTGACCGATATAAAGTTTTTTTTATTCCATAT |
| 757 | AACTGTCATGCCATTCGCCATTCAGGGGGGATGTGCTGCAAACGCCAGC |
| 758 | TCAGTTGTTTTTAGATTTCTACGTTTTTAAGAACTGGCTCACGAAAGAG |
| 759 | CGGTGTTTTTACAGACCAGGGCAAAAGAATACACTAAAACACTC |
| 760 | AAAACCAAAAGAAAAATAGGAATACTGGCTGACCTTCATTTTTTAAGAGTAATC |
| 761 | TCTGGTGCCGGAAACCATAATAGTAAAATGTTTCGACGATA |
| 762 | GCCAGTTAACAAAGTTACCAGAAGGGCCAGTGGGTGAATT |
| 763 | ACAAACGGCGGATTTTTTTACCGTAATGGTCGAGCTT |
| 764 | CGCATAGGGGACTAAAGCATGTCAATCATTTTTATGTACC |
| 765 | CTGAGGCTTGCAGGGAAACGAGGGATTGTATACGATGAAC |
| 766 | ATTCTACTAATTAAGGTAATCGTAAAACTAGACTTTTT |
| 767 | GTTAAAGGTTGTTAAACAAAAATACTGAGTAAGAAGCC |
| 768 | TTGCTTTCGAGGGTTGCAAGGCCCTAAAGGACGGAGTGA |
| 769 | CACCCTTCCTGTAGCGCTTCGGTTTATCAGC |
| 770 | TTCGCAAATGGTCGTTGATATTTTTTCAGAAAAGCCCCAAAA |
| 771 | GAGTCTGGAGCAAACTTTATCAACGAAAGACAGCATCGG |
| 772 | AATAAACAAAGCCTGTTTAGTATCTTAAATTT |
| 773 | TACCAGCGCCAAAAAACAGTCACGACGTTGTAAAACGAC |
| 774 | GTTATATACACGCCACCACCGGAAGCCACCCTCAGAGCC |
| 775 | ATTAATGCCGGAGAGGGTAGCTAATATGATATTTAAATT |
| 776 | AGCACCATAATCAGTATCAATAGATAAATAAGTATACAA |
| 777 | CAGCTTTCATCAAGGTCAGGATTACCAATAGGACAACAAC |
| 778 | TTGCCATCAGGGAGACAGTCAAATCACCATCATTTTTGAG |
| 779 | TTAAATTTCCGCTTTTATACCGATAGTTGCGCGCCTCTTC |
| 780 | TCTAAAGTTTTGTTTTGGGAATTAGATTTTTCCAGCAAAA |
| 781 | TACCAGAAGACTTTTTTGGATAGCGTCGCACCGC |
| 782 | ATTAGCGTTCGATAGCAAGAGAACCCTCATATATTTTAAA |
| 783 | ATAGTGAAGTAAAATACGTAATGCAGTTTCCATTAAACGG |
| 784 | TTGACAGGTTTTCATAGCCCCCTTTAAGGCGTAAATTCAT |
| 785 | ATCTTCTGCACCGGAACCAGACCTCAGAACCGCC |
| 786 | AGGAACAAGGAAACGTGATAAAAATTTTTCGCAGTCTCTG |
| 787 | AGTTTCAGATTGCGAATAATAATTTTTTCACCTCACCAGT |
| 788 | TTTATTTCCCGTGGGAGCGAGTAACAACCCGTGCCTTT |
| 789 | TGGTGTTTTTATTTTCATCGCGAGAACAGGGTGCCTCACCCAAA |
| 790 | TTTTCACGTCGTAATCTGCGCTCAAAAGCCTGAGCAAGCCGGTT |
| 791 | ATTGCTTTAGCATATAGAAGGCTTTATTAAACACAAGAATTGACAAT |
| 792 | AATTTTATCCTCCTTTTACATCGGACGCGAGGCCCATCCTCGGCTGTCGGCT |
| 793 | TTTGTTTCCTTATCATTCCATCAATAATAATTTACGTTTCATTT |
| 794 | TCCTGTTAAGCCGAGAATTAACTGAACACCCATCAGAGAATAAAAAC |
| 795 | TTTGGTAATTGAGCGCTAATTGAACAAATGAACCATAAACTTAA |
| 796 | GCCAGAATAAATTGTTCCTAAAGGTCAAGTTTCACTACGGTCAGAGGTTCT |
| 797 | AAAGAGGATTGACAAGATGGTTTAAGGTAGAGTAAAT |
| 798 | GAGAAAGGATCGCTGGCAGCCTTGGAACAAATAGGGTTGAGTGT |
| 799 | TTTGAGAACCGGATAACGAGTAAAGATTCATGCAGATA |
| 800 | CCACGCTGGTTTGCGTCGGTGGTGCCTTTTCACCTATTGGGCGCCAGT |
| 801 | CAAAGCGAATATCGCGCAAAAATCATTGAATCCCT |
| 802 | TCAGTTCCGGCCAGCAAGAATGAATTCGAC |
| 803 | TTTTTCATCCCACGTCGCACTCATCTAAAAAAACCCTCTATTAAC |
| 804 | TATGCTTTAAAATTACGAGGCATAGTCATAACGC |
| 805 | TAGCAACGCATGAGGACACTACGAAGGCACCAACCTAAAATTATACCAT |
| 806 | TACGTTGGGAATAGCGAACGCATAAAAAGCGGATTGTGGGAA |
| 807 | GTGTCTGGTTCGGTCGCATCGCCCGAGGCTTTTGT |
| 808 | TAAGTTTTGCCAGAGGGGGGGCAAAGCCAAAAAGATTA |
| 809 | TAGTAGGGCACGCTGAGGTCTGAGAGACTACCTCAGGTCA |
| 810 | TACATAACAACGCCAACCAGTATAAAGCCAACGT |
| 811 | TCAACATATAAAAGAAAATGTGAATTTCTTAATGAAACCATTGTTAAA |
| 812 | AATTGTAATTGGCGATTAAGTTGGGTAACGCCAGATACATAAT |
| 813 | AGTAATTATATAAAGTACCGACAGAATCGCCATATTAATT |
| 814 | AAAGGTAAATTCTTACATGTAATTTGGCATGATTAAAGT |
| 815 | TAGCAGAGAAAATACGGTTTTCCAAGGCTCCAAAAGGA |
| 816 | TCATCAGTAATAAGAGACTGTCCAGCCTTGAATAGGTTGG |
| 817 | ATGTTAGCCACCACGAAAAGAACTAGGCAGAGGCATTTTCGT |
| 818 | TCGGAATACCCGAATAAGTATATGCGTAATAAACA |
| 819 | CGCAAAGAAAACGTACTCCTTATTACGCAGTGGAAACGCAATAAAGTT |
| 820 | CTCAGGTTTTTGGTTTAGTACCGCACTATATGTATTTTAG |

H1 design oligo sequences (5′-3′)

| | |
|---|---|
| 821 | GCAGTAGAGTAGGTAGAGATTAGGCATGGCAATTTTCTGTATCTTTTGATGACTTAGC |
| 822 | GCAGTAGAGTAGGTAGAGATTAGGCAGGTGAAGGCCACGTCTTTCCAGACGCTAAACA |
| 823 | GCAGTAGAGTAGGTAGAGATTAGGCAGGGCGCTAGGGCGTATAGAGCTTGCGATGGCC |
| 824 | GCAGTAGAGTAGGTAGAGATTAGGCAACGTTGGTAAGAAACCTCACCAGTTAGCCCGA |
| 825 | GCAGTAGAGTAGGTAGAGATTAGGCAGCGGTCCGGCGTTGAGTAACATTGAGCGGAATTATC |
| 826 | GCAGTAGAGTAGGTAGAGATTAGGCAGACTTCAAACCAGACCTTTTTGAAGCAAACTCCAGCTGCGC |
| 827 | GCAGTAGAGTAGGTAGAGATTAGGCAGAATAGAACGGATTCTAACGCAAG |
| 828 | GCAGTAGAGTAGGTAGAGATTAGGCATGGCGAAAACACATTAAATGTGA |
| 829 | GCAGTAGAGTAGGTAGAGATTAGGCAATCACGTTGAAAATCTCCAAAGAC |

TABLE 6
Staple sequences used for the T3 triangle 2 folding.

SEQ ID NO:

Core structure oligo sequences (5′-3′)

| | |
|---|---|
| 830 | GTGCCCCCTGCCCGCTAGAGAGTTCCGGGGGTCCGTGGTGGTTCCGAA |
| 831 | AGAAAACAAAGAAGATGATGAAACCCCTGCCTGTACCAGGGATACAGAGCCAGA |
| 832 | GAGCAAAATTATTTGACGTATAATGATATAACTGAATTT |
| 833 | ACGGATTTCTTTGCTGCGGCTGGTAATGGGAAACATCAGAAAAATATGCTTTGA |
| 834 | GAATTATTAGTACCTTTTCAGGTTTAACATTGAAGCCT |
| 835 | TAGCAAGCAATAAAAGCCGTTTTTTTTTATTTTCTGCTGATGC |
| 836 | ATTTGAATTACCTTTTTTAATGGAAATTTTTAGTACATA |
| 837 | ACCCTCATAAATGGAAGGGTTAGAAATGAAAAAACGATTATTATTTA |
| 838 | GCACGCGAGCATTAATTGCGTTCCGGGCGAGCAAATCGTTAACGG |
| 839 | CGCGCTTAATGCGCCGCTACGAATCAGAATCGGAACCGGCGGGC |
| 840 | TGCGTAGAGCCACCCTACCGTACTCAGGAGGTAGCGGGGTTTACAAAA |
| 841 | ACATGTGGTGCTGCGGCTGAGAAGCGTGCTTT |
| 842 | TTTACATCACAGAAATCAAATAAGATAGCAGCTCTTACCA |
| 843 | TAAAAGAAACGGGGATGTGCTGCAACATAAAATAAATCAAGATTAGTT |
| 844 | CAGTACCTGGTGTATCCAGAACCGTCATTAACGCCACC |
| 845 | TACCGTAAAGCAAGCCCAATAGGATACTTCTGAATACGCACGGCCAGTGCCAAGC |
| 846 | ATCGTAGGAATCGTCAGATGAATAATCGGCTGGATAAGTCCTGAACAA |
| 847 | GCCATTCAGGCTGCGCAACTGTTGGGTTTTTAGGGCGATCGG |
| 848 | TTTCAGAGAACGTCAAAACCTACCATATCCCTACTATGGATCAGATGGGAATTAT |
| 849 | TGCCCGTACGGGGTCAGTGCCTTGCAATACTTTATCGGCCTAATCAGT |
| 850 | CATGAATTTTTGTATTAAGAGGCTTAACCTCCAAAGAACG |
| 851 | TCTTCGCTATTACGCCACGACGTTAGAGAATAAGGCGATT |
| 852 | CCGATTTAATATAATCGCCACGGGAACGGATAACCTCAC |
| 853 | AGGGCGAAAAACCGTCCGGAAACAAAGGAGCG |
| 854 | ATTGGCCTTGATATTCACAAACAACGCAGTCTGTATAGCTGCCGTC |
| 855 | TATCAGGGGGGGGAAAAGCACTAAGCGGGAGCAGACAGGA |
| 856 | CGATGGCCTTTGGGGTCGAGGTGTTCTGCCACGTTTTCA |
| 857 | ACAGGGAAGCGCATTATGCGGGCCAGACACTTTTTCACGGAATAAGT |
| 858 | CCCTCAGAACCGTTTGCCATCTTTTTTTTCATAATCAAAA |
| 859 | ATGGAAAGATAAATCCCCACCCTCAGAAAGCCACGTAAA |
| 860 | ACCGTCTGGTAATATCATACCTACCCTCGTTAAGGGCGCG |
| 861 | CAGAACAAACGGTACGGAGGCCACGCGCAGTGGTGTGTTCCGGTTGCG |
| 862 | GACGGGAGAATTAATTTTTTGAACACCCTGAACAAAGTCA |
| 863 | TCAATATTTTTAAAATTCAGCTGGCGAAAGGCAA |
| 864 | GACTTGCGCAAGAACGGGTATTAACTAATGCAGAACGCGC |
| 865 | ATACATGGAGTTTTAATAAACAGTTAATGCCTAAAGGTTCGCCTGAT |
| 866 | TAGAAGAACTCAAACCTTTGATTAAATTAACCGTTGTAGTTACACTG |
| 867 | CTGATTGTTTGGATTTCCTGATTTTGCTTTG |
| 868 | ACGCTAACGAGCGTCTACAGCCATTTTTGTTTCCGTGAGC |
| 869 | AATCAATATACAGTAAATACCAAGTTTGCTCAATTTCGGA |
| 870 | ACCAATTTTTGTACCGCACTCCGTTTTAGTTTTTGAACCTCCC |
| 871 | GGATTAGGATTGAGCCAGCTTTTTAAATCAC |
| 872 | CAACAATATCTTTCCTTATCATTCGGAGGTTTTACCGCGCCCA |
| 873 | TAGCCCCCACCTATTATTCTGAAATCAATTACTCGCGCAG |
| 874 | TTATTAGGAGTGTACTGGTAATACTTTTGATCGGATAAGCCGGAATA |
| 875 | AATCAATATCGACAATAAACAACTTTTTTGTTCAG |
| 876 | ATGTGAGTTTGGGTTATATTTTTTACTATATGTAAATATTCATT |
| 877 | TCAATCCGGCGCTCACTGCATCAGCGGTCATAGCAGCAAGCGG |
| 878 | GTATGAGCCGGGTCACGGATCCCCCGCCTGTGCACTCATAGAGAGGGT |
| 879 | TCGTCACCCCACCAGAGCCTTTTTCCGCCAGCATTGACAGGAG |
| 880 | AGGTCAGACGATTTTGACGCTCAAGAAGGAGCATGGCAATTCATGGCAA |
| 881 | CTGAGCAAAAATTAATTACATTTAACAATTTCCTGTTTATAGGCGAAT |
| 882 | CGGAAGCATAAAGTGTCATAGCTGACAGTTGATGTTGCCC |
| 883 | TCGTCATAAACATCCCAGTAACAGTGTTTTTATTGTCCAGTAAGCGTC |
| 884 | AACTGCATATAACAGTTTTCGCAAAAAAAGATGGTCTTTACAAAATA |
| 885 | AATAGGAATGTAGCCAGCTTTCATCAACATTGGCATAGT |
| 886 | AGACAATCTGGCCAAAAGAATACCTAACAACTATCTAACGGCCAGA |
| 887 | CTATTAGTCTTTGTGGCAC |
| 888 | CTGGAGGTGTCCAGCATCTTTTTGCGGGGTCATTGCAGGCGCTTTCGCAC |
| 889 | CCATCCCACGCAACCAGCTTACGGTAATGAATCAGCGTGGCCAGCG |
| 890 | CCACTACGAATACACTAAAACACGCCACGCTTAGAGAGT |
| 891 | TTGCTCCTCGCAGACGGACCCCCAATTAAACGGGTAAAATACGTAATG |
| 892 | ATCCTGTCCATCACGCAGTAATAACATTTTTCACTTGGAA |
| 893 | AGTACAACGGACTAAAGACTTTTTCATGAGGAAGTTTCC |
| 894 | CGATTAATTTTTGGGATTTTTAAACATTTTTGAGGCTTTGAGGA |
| 895 | GCGATTATACCAAGCGGTCCATTTTTGCGGATTAGCTCAAC |
| 896 | GGCTTAGAGCTTAATTTCATCTTTGTCAATCATAAGGG |
| 897 | TTCCAGTCGGGAAACCTTTTTTTCGTGCCAGCTGCAT |
| 898 | AGAGGCGGCGGTGGTGCAAGAATGCCAACGGCTCTAAAGCAGGAATTG |
| 899 | TTGCGGAACAAAGATTTTTACCACCATCGTCTGAA |
| 900 | CGCCATCAAAAATAATATTTTTGTATTATAGTACACGACC |
| 901 | AAAAATCATAAGAGGATTTACCAGGACAGATGAACGGTGTGAACGAGG |
| 902 | ACGAAAGAGCACATCCTCATAACGCCGTTTTTTTAGAGCCTATTAGAC |
| 903 | AACCGACTCAATATCTTGAGAGCCACACCGCCTGCAACAGTGCTGAATA |
| 904 | TAGAACGTCGGCCAACAGTGAGACGGGCAACAAAAAGAAT |
| 905 | ATTGCATCATGTAAAAGGGACATTATTTTTGAATGG |
| 906 | AAGTTTCAACCTTTAATCCAACAGTGAAAGAG |
| 907 | TTCATAGCCCTAAAACATCGCCATAGTATTAAGCAGCAAATGAAAAA |
| 908 | CCGAACGATCTGACCTGAAAGCGTCAGAGATATTTACAAACAATAAG |
| 909 | CGAGTAGTAAATTGGGCTTGATCAAGAGTAATCTTGGGCTTTGA |
| 910 | AAACGATGCTGATCCCAGCCCGAGGAGTCCACTATTAAAG |
| 911 | ACGATCCACGAGTAAAAGAGTAATATCTTTTTGGTCAGTTGG |
| 912 | CTTATAAATCGCTGATTGCCCTTTTTTTACCGCCTGGCCCTG |
| 913 | CAGCAGGCTTTTTAAAATCCTGTTTGATAAGCCGGCGAACGTGGC |
| 914 | GTGCCGGCCGGGTTTTTTTCCTGCAGTGCTGGTCTGGTCAGC |
| 915 | GCTGAACCTCAAATATCAAATTTTTCCTCACATCAGAT |
| 916 | TCGTCTCGCCTTTAGTGATGAAGGTTGGGCGGATTAAATCCTTTGAACCACCACACCCG |
| 917 | TGGATTATAACAGGAAAATTTTTCGCTCATGGAA |
| 918 | CCACGCTGGTTTGTGCCGTTCCGGCATTTTCACCGCGCGGGG |
| 919 | ATCACCTTGATAAAACAGAGGTGAGGCGGTCTAAAAATAAGGAAGGTTAATAGA |
| 920 | TATTACTTTTTGCCAGATAATACACAAATCAACGGACTTGAGCAACCG |
| 921 | CGCCATGAACGTGGACTTTTTCCAACGTCAAATCGGCAAAATC |
| 922 | ACGACGATAAAAAAAAAAGCCGCCAAAGCGGTAGGAGCAAATGCGCG |
| 923 | GAACCCTTTACATTGGCAGATTTATCATTAATTTTAAAAGTTTGCTCACGGA |
| 924 | ACAGGCGGTCGCTGGCAGCACTGACCAACTTGTCAGGAT |
| 925 | TCCCGGAATTTGTGAGAGATAGACTTTCTCCGCGCAGAAA |
| 926 | GTGTAGCGGTTTTTCACGCTGCGCAAACTTAAATTTGGG |
| 927 | TTGTGTACAAAAGAGATGGTGAAGGGATAGCTTCGCGTCTGGCCTTCC |
| 928 | GTCAATAGCCATTGCACCACCAGCACCTGA |
| 929 | TTTACCAGAAGAATTTTTAGCGAATCGGCGAAACGTACA |
| 930 | CTTTTGCATACCAGTCAGCCCGAACAGACCGGAAGCAAAC |
| 931 | AAAATCTACCAAAAGGAATTACGAAAATGTGATACTGCGG |
| 932 | GCGAGTAACAACCTAACGCGTTAATAAAACGAACTA |
| 933 | ATCGACATAAAAAAATCCCGTAACCCCTGACTTAAATCAGTTAAAATT |
| 934 | ACTTTAATTTTTTCATTCTGACGAGCCAAATCAACGTAACAGCAGCGAA |
| 935 | GCGCATCGTAACCGTAATTGCGACGATGAACAGAGTCTGGAGTAGA |
| 936 | AAAGAAGTTTTGCCAGATTTTTGGGGTACCTTATGCGTTTTTTTTT |
| 937 | TTTAATTCAAAAAGGCTTTTTCACGTTGAAAAATAGTAAAATGAGATGG |
| 938 | CGATTGAGCAGTATGTCTTTCCAGCTGGTGCCGGAAACCAGGCAAAG |
| 939 | ACCAGAGCCATCGATAGGCCGGAATGGTTTGA |
| 940 | GCTTAGATCAGGGAGTTAAAGGCCCGGTCGCTGAGGCTTG |
| 941 | CTTTTTCAGTAATCAGTAGCGACTCCTCAAGAGA |
| 942 | AATCCAATCGCAAGACGGCTTAGGGAATAACCTTGCTTCTGTAAATCG |
| 943 | ATCAGGTCCGGTTTATAAGGAACAACTAAAGGGCATCTGC |
| 944 | GTAAACGTTTTTTAATATTTTGCTCATTTTTTATATTCATTGAATC |
| 945 | TCTCCGTGGGAACAAACAGGAATACCACATTCAACATTATT |
| 946 | AACAGTACGGGAGGGAAGACAGCCCCTACAACG |
| 947 | CATTCAACATCACCGTATGATTAAAATACCGAGAAAAAG |
| 948 | AGTACAAATCATAGTTAGCGTAACGATCTCAAAAGGGCGA |
| 949 | AGTAAGTCAATAACCTGTTTATTTTTCTATATTTTCTTTAGTTTGACCA |
| 950 | GCGCGAGCGCAAATATCCAAAAACAGGAAGATTTTCT |
| 951 | TAATATCAGTAGAAAAGAACTGGCCACCGACTCCATTACCAGAGCCGCC |
| 952 | GAGCCACCACCCTTGGTTTACCAGCGTTTTTCAAAGACAA |
| 953 | TCACCGGACGTTTTCATGCCTTTAGCGTCAGAATCAAAATTTAGTTAA |
| 954 | TAGATACATGATTCCCAATTTTTTCTGCGAACGAGCAAACCCGTTCTA |
| 955 | TTCTAAGAACGCGAGGATCGAGAACAAGCGAAAATTCTGTCCAGACGA |
| 956 | TAAAACTAGCATACGAGAATAGACAGCTTGCTTTACCTAAATTTAA |
| 957 | TACCCCGGTTGATACAGTTCAGAAAAGAGAATATAATAAT |
| 958 | TCCAAAAGGAGCCTTTACCGATAGGCTGATAAAATGTGTA |
| 959 | AATTGTATATTGCCTGGGTAATCGAATCATAATTTGGG |
| 960 | GCGTTAAAATCAGATATAGAAAAATACATACATAAAGGTGGCAACAT |
| 961 | AGACTTTTTTTAAATATCGCGTAACGAGAATTTTTGACCATAAATC |
| 962 | CGGAACAACTAATTTTTTGCAGATACACGTCGGA |
| 963 | TTAGCAAAAAAGGCTGTAGCTAGGTGAATTTGAGAAGATAGCGATA |
| 964 | CATTATGACCTGCAACTATTTTTAGTACGGTGTCTGGTAATGCTG |
| 965 | ACGTCACCTCATTAAACGCACTAATAGTAGTAGCATTAAC |
| 966 | GGTAAAGTTAAACACCGGAATCATCCAGGCTT |
| 967 | CGACAAAACGAGAAAATAAATAAGCAGTAGCATGAGCCATTTGG |
| 968 | ACAATGACAACAACCATCTCCAAAGAGCTTTTTTTAAAGCGAAC |
| 969 | TGTTTTAAATACTGGGTAAAGATTCAAAAGGCATAACC |
| 970 | TTCTGCGATTTTTGAGCCCTCATAACGCAAGGATAAAAATCTGTAGCG |
| 971 | TCAGAGCCACCACCCCTCCCTCATTAGCAAGCAGCACCAATATATT |
| 972 | ATCCGGTAGAGGGTAATTGAGCGCCGCCATTCTTATTTTG |
| 973 | CATAGGTCTAATTAATTTTCCCTTAGAATCCTGTATAAAGTTTCATC |
| 974 | GCCTGATATCGCCCACGGTGAGAAAGGCTTTTTGGAGACA |
| 975 | TGGGATAGTATCGGCCTCAGGAAGGAGGGGAC |
| 976 | ATTCCACAGGTAAATATGGCATCAATTCTCACCAGAACCA |
| 977 | TAATACTTTTGCGGGAGAAGCCTTCGGTTGTCGGAGAGG |
| 978 | GTATGGGTTTATCGCACTCCAGCCAGCTTTCCGGGTTAAGCCACTTTC |
| 979 | ACGGCTACAGAACAAGAACTTTTTGGATATTCATTACAAACACCAGA |
| 980 | GGAGATTTTTTTGTATCATCAGACAGCATCGGAACGAGGGTAGC |
| 981 | ACAGGTAGGGCTTGCCGTGAATTAAATTGTGTCGAAATCTTTTTGCGACCTGCT |
| 982 | GAAATTATAGATCTACATTAAGCAATAAACACCGGAACCGCTCAGAGCGGTGAATT |
| 983 | TACCTTTTGAGACAGAATCAAGTTTCGGCATTTTCGGTC |
| 984 | AATGAAACCACGCCTCAGAGCATAAAGCTAAATTATTTCA |
| 985 | TTTGATAAGAGGTAAATCACTTTTTATCAATATGATATTCAA |
| 986 | CCCTCAAATGCTTGTCACGTTGGTGTTTTGACCGTAA |
| 987 | GCCTGAGTATTAATGCACCAAAAACAGGCAAGGCAAAGAAATCCAATA |
| 988 | TATTTTAAATGCAATTAAGACGCTCTTAAACAGCTTGAT |
| 989 | ATATCCCATCCTACTCTTTCCTGTGTGAAATTGTTATCCGCTCACCTGT |
| 990 | TGCCAATTCCACACAACTAGAAACCGCTATTTTAAAGAAATAAGTTGGG |
| 991 | GGGAGAACTCATTTACGAGCATGATACGAGCTCCCAATCGCACCCAGCTACAACTT |
| 992 | TTAGATTTTATCCTGAACTTTACAGGTAAAACGCAGGGTTT |
| 993 | TTTTTAACTCACTCGAATTCGTAATCATGGTAAAGCCTGCTTCGCGTCACCCAAA |
| 994 | TCACTGCGGGGTACCGTGCCTGTTGGGTGCCTAATGAGTGAGCTCAT |
| 995 | TTTGGCCAGTTACAAAATAATTCCAGAGTGAACCATGTGGAGCC |
| 996 | CCAGAATCCCAGAATGCCTAAAGGTCAAGTTTCACTACGCCTAATTTTTCT |
| 997 | TCTTGGCTGGCTCTTAGCCGACAGACCATAATTTCAAAGAACTG |
| 998 | CGAAACAACCATGTTAGACCTTCAGATGGTTGGCGCATATCCT |
| 999 | TGAGATCATAACCCTCGAGGACGTTGGGAAGAGCTCATTA |
| 1000 | GGCAAAAGAAGGCACCAACCTAAAAGCACCGTTTTGCGTATTGAAGT |
| 1001 | TAACGGCGCCAGGGTGGTTTTTCAACGCGGTGAACGTGCCAGTTGAA |
| 1002 | AGAAAGGAACTGCTCATTTGCCTGGAACAAATAGGGTTGAGTGTTGACCT |
| 1003 | TAACTTCCAGTTGCCAGCAGGTAAAGTTTTTAGAAG |
| 1004 | TGTCTGTAATTTAGGCATAAAGTACTCGCTATTGAGAGAC |
| 1005 | GAGAGATAATAATAACCAGAAGGCCATATTTAACAACGCCAACAAGGT |
| 1006 | TCAATTACGGAATACAATTACTACCGTGTGA |
| 1007 | GACTCCTTGAAACGCAACCCACAAGCAGATAGCCGAACAAAGAAGT |
| 1008 | GAGAATAGAGGCATTTTCGAGCCCCAACGCTCAACAAACT |
| 1009 | TCTCGTAGGGCTTAATTGTATCATATGCGTTATT |
| 1010 | AGTAATAACCTGTTTAGAGAATCGAAACCGAGTACCGAAGCCCTTTGGT |
| 1011 | TAGGTTTAAGAAAAGTAAGAATTGACACCGCTTACGTTAGTAAATGAA |
| 1012 | TTCTTACCATGAAAACAGTCAATAGTGAATTTTTTTAGAA |
| 1013 | TAAACAATGAAATAGCAATAGCTATCTATTTTCAGCGGAGTG |
| 1014 | TTGGTCAATCATATGCAGATTTTGCTAAACACAATAATAAGT |
| 1015 | TTGCGCCGGATATATTGCTTTTGCGGGATCGTCACCCTCAAAGCTGCTT |
| 1016 | TAGCTCAGTGAATAAAAAGATTCATCTAAT |
| 1017 | TTGAGATTTGGCGGATTAGACTGGATAGTTTTTGTCCAA |

H1 design oligo sequences (5′-3′)

| | |
|---|---|
| 1018 | GCAGTAGAGTAGGTAGAGATTAGGCACATCATATAACCCATGGTTGAGGCACGCCGCC |
| 1019 | GCAGTAGAGTAGGTAGAGATTAGGCATCCCAGTCATACATTTTCAGGGATCACTGAGT |
| 1020 | GCAGTAGAGTAGGTAGAGATTAGGCAGGCGCTAGGGCGCTCAGAGCTTGA |
| 1021 | GCAGTAGAGTAGGTAGAGATTAGGCACGCATTAAAGTAACATCACCAGTCCAGTCGACAACTCGT |
| 1022 | GCAGTAGAGTAGGTAGAGATTAGGCACAGCGGATCGTCCCGAACGTTATT |
| 1023 | GCAGTAGAGTAGGTAGAGATTAGGCAAATCGTCATAAAACCGCGAGAGGAAGAGCAACACT |
| 1024 | GCAGTAGAGTAGGTAGAGATTAGGCACCTGTAGCTTGTATAATGAAAAGG |
| 1025 | GCAGTAGAGTAGGTAGAGATTAGGCAGACGACAGTAAAATCAGAAAAGCCTTAAATT |
| 1026 | GCAGTAGAGTAGGTAGAGATTAGGCATCAAAAGTTTTGTCGTTAGCAAAC |

TABLE 7
Staple sequences used for the T3 triangle 3 folding.

SEQ ID NO:

Core structure oligo sequences (5′-3′)

| | |
|---|---|
| 1027 | AAAATCCTGTTTGAAACAGTGTAGCGGTCACG |
| 1028 | AACAAAATTTTCAATTTTTCAGGGACACTGAGATGATACAGGAGGT |
| 1029 | ATCAAGAAACGCGAGGCGTTTGCTATCCGGTATTCTAAGAGCACTCAT |
| 1030 | GCGCATCGGGAGAAATTTTAGACACCCTCAGTGAGTAAC |
| 1031 | TTCCCTTAGAATCCTTGAAAACATAGTTTTTGATAGCTT |
| 1032 | ATCAATATATGTGAGTTTTGCTCACACCCTCATTTCGGAA |
| 1033 | TTGAATACGTACCGTAATAGCAAGCCCAATAGGGAGGTTTTTTAACAA |
| 1034 | AAACAAACGTTTTCACGCTGCGGCCAGAATGCGAATAACCCAAGTACC |
| 1035 | AGAAGGATATTAAGAGGCTGAGACGCTGGTAACATGGAAATTCTTTGA |
| 1036 | CACCGTACTCAGCAGCACCTTTTTTAATCAG |
| 1037 | TCGATAGAACCCATCAAGTTACAAAATTGCAATAACAT |
| 1038 | GGAGCGGGAAGAAATTCAGTTGGGCGGTTGTGTACATCG |
| 1039 | GCCACGGGAACGGATAACCTCACCGGTTTTTAACAATCGGCG |
| 1040 | AAACAGCCATAACCTAACGAGCGTTTTTCTTTCCGTTTGAAAT |
| 1041 | CGCCTGTAGCATTATTATTCATTAAATTTTTGTGAATTAT |
| 1042 | AAAAGAAGTGCTAGCGAACCTCCCATTACCGCGCCCAATAGCAGGT |
| 1043 | CCGGGGGTTCCTCAAGAGCAATACTACCCGTATAAACAGT |
| 1044 | GCGTAGATTTTCAGGTCAAAATTGCCGATTA |
| 1045 | TTCTGCCATTCCTGTGGTCGGGAATGAGCTAACTCACATTCACCCAAA |
| 1046 | GGTAAATTTTTATTGACGGTCCCGGAATTTGCAA |
| 1047 | ACCTGAGCGATTCGCCGGAGAATTTTGAGCGCTAATATCAACAGCGGATCTCACGG |
| 1048 | AGACGTTACAAATAAATCCTTTTTCATTAAAGCCAGAATGGAA |
| 1049 | CTCTGAATTTACCATAAAAGGGACATGGAAGGGTAAAACAGAAGGGCGC |
| 1050 | GTAGTTCCAGTAAGCGTCATACATTAAGTTTTAGCCACCACCCTCA |
| 1051 | GCTCATTTCAAAGTCACAGTACCTTTTAAAAAACAGGAGATTTGCACGTTAGAAC |
| 1052 | AGCGCCATGTTTACCAACGCAGAAGAGAGATATTCTCCGT |
| 1053 | AGCATTGACCACCCTCAGAACTTTTTGCCACCCTCAGA |
| 1054 | AGGAGTGTACTGGTAAGGCTTTTGTTTCGTCACCAGGGACAATAACG |
| 1055 | AGTGCCTACATTTTGACACGACCATACGCCAGGGGAGCTA |
| 1056 | AGCGCCAAAGATGAGAGATAGACTACCCACAAAAAAACAGGGAAGCGC |
| 1057 | AGGGCGAAAAACCGTCACATAAAACGCGCTTA |
| 1058 | TATCAGGGCGCTGGCAGTGGCGAGGAAGTGTTAGAGTCTG |
| 1059 | CGATGGCCTGACGGGGAAAGCCGGGGGTGCCCCGGAAGC |
| 1060 | GAATTGAGTTAAGCCCAAACGTACAAGGGCTTTTTGACATTCAACCG |
| 1061 | AATAATAAGAGCAATTTTTAAACAATGAAATAGCAATAGC |
| 1062 | CGCTCAATTCCATCACTTAGTAATATAGCTGTGCACGCGTCAGTGTCA |
| 1063 | TAATGCCCATGAAAGTTAGGATTAGCGGGGTGGCGGGCC |
| 1064 | AACAGGAAAAACGCTTATCCAGATCAAACTATCGGCCTT |
| 1065 | TTCCTCGTTAGAATCAGAGCAATCCTGAAAAGGAAGCATACGAG |
| 1066 | CTCCCTCACGGATAAGTGCCGTCGTGGAAACATTTCATTT |
| 1067 | GAGCCGCCCTATTATTCTGAAACCCTGCCTAGAACCGCCACCCTCAT |
| 1068 | TAATGAGACCTGTCGTGCCAGCTCCAGCGGCCTGTTCTTCGCGTC |
| 1069 | TAGGAATCGACTTGCGATTAGACGTGATTGCTGGTGAAGG |
| 1070 | ACCCTGAAAATTGCGTATGCCGCTCACAATTACCGTTGTAGGAACGG |
| 1071 | CAGCCTTTGCTATTTTGCACCCAGTCAATAATCGGCTGTC |
| 1072 | AGTTTTGTTCATAGTTAGCGTAATTTAACGTCAGATAGCTCAAACTTAAATTTCT |
| 1073 | GTACATAAGTAAATCGTCGCTATTAATTAATTTTTCCTTAGAATTACC |
| 1074 | TGAATCGGAGAGAGTTTAAAGCCTGCGTGGTGGTTCCGAA |
| 1075 | TCAGACGATGCATTAAATTCGTAAATAAAGTGGCAGCAAGCGG |
| 1076 | GGAGGTTTATTATTCATAATTACAAGTACCGCGTACCAGG |
| 1077 | CTACATTTTTATTTTATCCTGCGTCAAAATTTTTTGAAAATAG |
| 1078 | AGATTAAGATTTACGAGCATGTATTTTTAAACCAA |
| 1079 | CGCTGAGATCATCTTCTGATTTTTCTAAATTTAATGTTTTTTAA |
| 1080 | CTGCGCGCCTGTGCACGCTTTCCATGAAATTGTTATATGGAACCGCC |
| 1081 | TCATTCCATAAATCAAGATTAGTTACAGAGAGCAGTTACAAAA |
| 1082 | CGTCAGCGTGGTGCTGGTCTGGAAGGAAGCAAGCGCATCGGATAGGTC |
| 1083 | CTGCGAACGAGTAGATGCGACCTGTCAAGAGTAATCTT |
| 1084 | GTTTGGATTATACTTTTTTCTGAATAATTCTGGCC |
| 1085 | GACAAGCGCTTCGACAACAACAGTTTCAATATCTGGTCAGTTTAGTTTG |
| 1086 | TTTGGGGCGTGTCTGGTTTTAAATATTACCCA |
| 1087 | GTACTATGGTTTTTTGCTTTGACGCCTCCGGCCAGACCA |
| 1088 | GCGCAACAGTGCCACGCTGAGAGCACCCTCAAGAAAGGAATTGAGGA |
| 1089 | TTTCGCACCCTGATAAGTACAACGGAGATTTGCATCCCTT |
| 1090 | TGCCATCCCACGCCCCAGCCCGAGGAGTCCACTATTAAAG |
| 1091 | CAAAGCGATCCTTTTGATCGTCATAATCAACGTAACAAAGGGCTGGCT |
| 1092 | TCAGATGCCGGGTTACCTTTTTTCAGCCAGCGGTGCCGGTGCCCCCTGCA |
| 1093 | AAATATTCATTGTCAGCAGCAACGGATTAGACTCGTATT |
| 1094 | GTGTTCAGCAAATCGTTAACGGCAGCGCCAGGGTTGCCCTTCCCCG |
| 1095 | CCACGCTGGTTTGAACCAGCTTACGGCTGGAGGTGGTTGCGG |
| 1096 | ACACTGGTCTTTGCTCGTCATAAAAGGTTATCGAAGTATT |
| 1097 | CTTATAAATCGCTGATTGCCCTTTTTTTACCGCCTGGCCCTG |
| 1098 | CGCAAGAAGTCATTGCAGGAACCGGATATTCATGCAACTGAGTACCT |
| 1099 | GAAACAAAATTGTGTCGAAATCCTGGCAAATAAAGTACG |
| 1100 | GCAGCACCTGCCGGACTTATCATCATATTCACGTATAACGTGC |
| 1101 | TGAACGGTGTCGGTCAATGCAAAAGAATACACTAAAACACTC |
| 1102 | TAAAAATATTTGAATGCCTAAAACAAATCCTCAAACAA |
| 1103 | CCAACGCGCGGGGAGAGTTTTTCGGTTTGCGTATTGG |
| 1104 | AAAACAGATGAAAAGGCAGGCAAGTTAATTGCACCAGACCTCCCCCT |
| 1105 | TATCATCGTCAATCCGCCGGGCGCGTCCAGCAACGTTATTGCGGAACA |
| 1106 | AGAGGGTTTTTTGATATAAGTATATATATTTTTAAGGCGT |
| 1107 | TTAAACAGACTGCGGAATAAGAGGGCTGTAGCTCAACATG |
| 1108 | TTATGAGTAGAAGAACACAATATTACTTTTTGCCAGCTCA |
| 1109 | AACTTTTTTATAGACGTGAGCCGAGCTCGA |
| 1110 | AAATCTACGTTAATAAAACGATAAATTGGGCTTGAGACGAAAGAG |
| 1111 | TATTATCTGGTGCCGGAAATTGCCAGATACATAACGCCAAAA |
| 1112 | TTAGTGATGAAGGGTAAAGTTAAACGATGCTGCGTCTCGT |
| 1113 | AATTTTAAATTATTTATCTAAAGCACATTG |
| 1114 | CAGCAGCAAGACTTTATTGCCCGATCAGCGGGTGCCAACG |
| 1115 | AATGAAAATGCGCGAACTGATAGCGCTATTAGAAGAAACCACCATCA |
| 1116 | AGGATTTATAAAATATCCTTGCTGAACCTCAAATATCAA |
| 1117 | AAGTTTCAGACCTTCACTCCATGTACCCCCAGCGATTATACCAAGCGC |
| 1118 | TTAACACCGCCTGAGCGGTGAGGCACCACCAGCAGAAGAT |
| 1119 | CGTCTGTTTTTAATGGAAGTTTGATACATTTGTATGAGCCAAAGGTTT |
| 1120 | GGTACCTCCTCACATTTTTTTGAGGAGCGGCTGG |
| 1121 | GAGGCCATTTTTCGAGTAAATTTATATTTTTTCAGTGTAACATT |
| 1122 | ACAGAGATACATTGGCAGTTTTTTTCACCAGTCA |
| 1123 | GGCGGCCTCCACATTTTTCCCGCAAATCCCGTAAAAAAA |
| 1124 | TTGTAGAACGTTTTTTATTGCCGTTCCGGCAAATCGGCCTCAGGAAGA |
| 1125 | CCGCACAAACGTGGACTTTTTCCAACGTCAAATCGGCAAAATC |
| 1126 | GCACACAGACAATATTCCGAACGAGGTCAGTAATCGCCAT |
| 1127 | TCTTTAAAGAACCCTTCTGACCCCTGATTTGATGGCAATTCATCACGCGGTC |
| 1128 | ACACCAGAGGCGCATACTGCTCATGAACAACATAGGAATACCACAAAT |
| 1129 | TCATGGTCAACATCACTTGCCGAGCCGTTTTTCAATAGATAA |
| 1130 | AGGGGACGACGACAGTGTAGATGGACTCCAACTAAGAATA |
| 1131 | GAATAAGTAACTAAAGCGGCCAGTGCCAAGCTTTCAGAG |
| 1132 | TCATTTTTTTTTGCGGATGGCTTCGCGTTTTTTTTAATTCGAGCTT |
| 1133 | CCAACCTAAAATGGTTTAATTTTTTTCAACTTTAATCGTTGGGAAGA |
| 1134 | AAGAGGAAGCCCGCGATCGGTGCGGGGGTCAACTGTT |
| 1135 | GCTCCAACGGGGAATTAACAACTTTATTTTCT |
| 1136 | CCGACTTGAAGGCCGGAAAGAAACTACAAATTTTTAACA |
| 1137 | GGGAAGGGTAAGTTGGGTAACGCCTGTGCTGC |
| 1138 | CAGCTTTCATCAAGACTTCAAATACCAATAGGTGAATTTC |
| 1139 | GTAAATGACAACAGTTTCAGCGGAGTGAGGAACACCGTCA |
| 1140 | CGTGGAAGAATTAGCAAAATTTTTTTAGCAATAAAGCATTAACATCCAA |
| 1141 | GCGGATTGTTTTTCCGTAATGGTAACCGTGCATGCATCAAAAAGAT |
| 1142 | AAATCATATGGCATCAATTTTTTTTACTAATATTTTTTAAACAGGAAG |
| 1143 | CACCATTATTGCATTATGACCCTGTATTTTAATCAAAAGGTTTTAGAA |
| 1144 | ACCAGTAGTTGTTAAAGTGAGAAAGGCCGCCAGTAAAGATATGCAATGCCTGAGTA |
| 1145 | GAGGCAGGTCAGACACCACCAGTTTGCCTTTTTTCGGTCCGGAATC |
| 1146 | GCATAAAGCCGTGGGAGCGAGTAACAACCCGTATTTTT |
| 1147 | TTAAATTTATAACCGACGGTTTATCAGCTTGCTGGCGAAA |
| 1148 | TTATATAATTGAGGACTAAAGACTAACGGCTACAGAGGCT |
| 1149 | TTTTTAAGTCACAATCAACATATAAAACGTCAGAATCAAGAGCCGCCGC |
| 1150 | TCCTGAACTAAATAAGTATAAAGCTAGCGACACCAATGAAACCA |
| 1151 | ACGATTTTTTGTTTAAAATCTTACCAACGAGTTAATATCCCATCCTAA |
| 1152 | TTCCATATAACAGGTTGATATTTTTTCAGAAAAGCCCCAAAA |
| 1153 | ATAAGAATATCTTACCGAAGCCCGTGGAGCCATTGAGGG |
| 1154 | AAGAAAAAAGGGCTTAATTGAGAAGGTATCCAA |
| 1155 | TTCTAGCTGATAACCTGTTTTTTTAGCTATATTTTCAACCATTAG |
| 1156 | TTTGCTAAGAGCCAGCGTTGTACCAAAAAATATTCACAAA |
| 1157 | TCAGGTCACGCGTTTTCCGGAGACAGTCAAATCACCATCATTTTTGAG |
| 1158 | GAGTCTGGAGCAAACCTATATGTCAGGGAGTTAAAGGCC |
| 1159 | AATGACAACAACCATCGCTTTTGCATTGTATACGATGAAC |
| 1160 | TTATTACTTTTTAGGTAGTCAGGACATTGTGAATTACCTTAACGGGTAA |
| 1161 | GCCCACGCTTGTTAAACAAAAATAGAAGCCTCCTCAGA |
| 1162 | CAACGCTCATTATTTATCCCAGGCAATAGAAAATTCATATGGTTTAC |
| 1163 | AAACCTTCCTGTAGCGGGGAAAATCTCCAAAAAAAAG |
| 1164 | AGCGCCATTCGCCATTCAAAATAGCGAGAGGCTGAGGCATA |
| 1165 | GCCTTAGCGTTTGCCACCACCACCGGAACCG |
| 1166 | CCCTCATATAATACTTATAAAAAT |
| 1167 | CACCCTCAGCAGCGAACGATAGTTAATTGCTTTTTGAATATAAT |
| 1168 | ATGTGTAGCCAGAACCGATTGGCCCCATTAGCAGCCATTT |
| 1169 | AGCCTGCTTTAATATTTTGCCTGAAGATCTACAAAGGCTAATCACCGG |
| 1170 | AATAAACACATAGCCCCCTTACGGAATAGGTGTA |
| 1171 | CCGACCGTGTGATAAAAGTTAATTAGAGTCAATAGTGAATTTATCAAA |
| 1172 | AAGAGAATAGCAAATATTCAACCGTTATTTCAACGCAAGGTTGCGGGA |
| 1173 | AGAACGCGGTCTGAGAGACTACCTTTTTAACCTACCGACATAGAAAA |
| 1174 | CGCTATTACGCCAGCTTTCGAGGAACGCCATTCAGCTCAGTAGTAG |
| 1175 | TTCAGAAAACGAGAATGTTTTTCCATAACATCAGTTGTTTTTGATT |
| 1176 | ATTAATGCCGGAGAGGGTAGCTAATATGATATTTAAATT |
| 1177 | CATAATTTTTGGAACCGAACAATACGTAATGCCACTACGAAGGC |
| 1178 | TGACCAACAGACAGCAGCATGTCAATCATTTTTATGTACCGGCGCAGA |
| 1179 | GTAAGAGCTTATACCAGAAAGATTTTTGAAAGAGGACAG |
| 1180 | CAAATGGTCAATAAGGTAATCGTAAAACTATCGGAACG |
| 1181 | GAATTACTTTGCTTTTTAAAAGAAGTTCCAGGCA |
| 1182 | ATTCGCAGTAAACGGAGGCTTGAAATGCTGAGGTTGGG |
| 1183 | GCCACCACAACCAGAGTCTTTTCATAATCAAACAAGACAAATAATTAC |
| 1184 | CCTCAGAGCATCGGCATAGCGTCAATGCGTTAGCAAAGAC |
| 1185 | AGAGCCTAATTCGCGCAGAGGCGATGAAGCCTAGAACGGGAGCAAGCCAGGT |
| 1186 | TCATGTTTTTATTTTCATCGCGAGAACATATTAAACTTGCTTCT |
| 1187 | ACTACAAGATGAATATACAGTAAGAGGGTAAAACTGAACCGAGGTGCTCGT |
| 1188 | TCCCCGTAAAGCACTAAATCTTTGGAGCAAATCAGGGCT |
| 1189 | TTTGATATAGAAGGCTTCACTGCCCTCTGTGGTGGTCATA |
| 1190 | TTTGCCCCCGATTCAAGTTTGGAACCCTTGAACCATGCCGCCAG |
| 1191 | GCAAATTACCACACAAGGAAGAAATTAGAGCTCACTACGAAAGGGAGTTCT |
| 1192 | TACGAGAAATCTTTGTACTTAGCCGGAACGACCGTTGATTCCCAATTATACATTTC |
| 1193 | ACAGACCAACGAGTAGACTAACGTCAGTGAATAAGGCTTT |
| 1194 | TGGATAGCGTCCTTCAACTAATGCAGAGGGGGTAATAGTACCGCTTAGTCAGA |
| 1195 | CCAGTTTGTCGCACTCCAGCCAGCTTTCCGGCAAAATGTTT |
| 1196 | CTTTAGGAGCACTAACTAATGGGTGGGTCACTGTGGTTTTTCT |
| 1197 | TCCAGTGAGACGGGCAACAAAAAGAATCAGCAGGC |
| 1198 | TGCGCGTAAGCACATCCTCATATGGAACAAATAGGGTTGAGTGT |
| 1199 | TCAGTTACGGAACGGTCGGTGGATCATTTT |
| 1200 | GGGATCGTAGGGTAGCTTTTCATGAGGAAGTTTCCATTAATGCGATTTT |
| 1201 | TCTGGCTCAAACACTATCTTGATACATCAAAAATCACCTCTT |
| 1202 | TAGAGCTTGCGCCGACTTAAACAGCATAACCCTCT |
| 1203 | TCAGACGACGATAAAAACCAGGCTGCGCTTTACCCTGA |
| 1204 | TTAATAAGAGAATATAAAGTCCGGCTTATGCAAATCCAATCG |
| 1205 | ATTCGCGTCTGGATCAATTGTATTATATTCGGGCATTTTCGT |
| 1206 | TACGCAGTATAGGCAGAGTCGTTTAGTATCATGACTGTAG |
| 1207 | TCACGTTGGAAGGGTTTTCCCAGTCACGACGTTGTAAGACTCT |
| 1208 | TCAACAAGCTAATGCAGAACGCGAAAGGTAAAGTAAAATT |
| 1209 | TTGTCCAGACGATGTAATTTGTTAGCAACCAAAGGAGCCTTT |
| 1210 | CCTGTTTAACGCCAACACGACAATAGGAAACCT |
| 1211 | TTACGAAGGCATGATTAAAACGAGAATTGCGAATAATA |
| 1212 | TACATCATAGATAAGATCATAGAGAAAACTTTTTCAAA |
| 1213 | ATACCCAAAAATACATTACCAGAAAACAACAT |
| 1214 | TCCGAACAAAGTACATAAATCGCCATACTTACCAG |
| 1215 | AACGTAGAAAGAACTACGCAATAATAACGGAAAAAGTAAGCAGAAACT |

H1 design oligo sequences (5′-3′)

| | |
|---|---|
| 1216 | GCAGTAGAGTAGGTAGAGATTAGGCACTACCATACGATCTAAAGCGCAGTAAGTACAA |
| 1217 | GCAGTAGAGTAGGTAGAGATTAGGCAAAAAAGAGGAACCACAGACAGCCCCGTCTTTC |
| 1218 | GCAGTAGAGTAGGTAGAGATTAGGCAATGCGCCGCTACAATACGCTAGGG |
| 1219 | GCAGTAGAGTAGGTAGAGATTAGGCAACGTTGGTAATATAATTGAAAGCGAGGGAAGGAGCGGAA |
| 1220 | GCAGTAGAGTAGGTAGAGATTAGGCACGCTGGCAGAGCCTGATTATCAGA |
| 1221 | GCAGTAGAGTAGGTAGAGATTAGGCAAGCAAAGCGGATTCTGCAAATGCT |
| 1222 | GCAGTAGAGTAGGTAGAGATTAGGCATATGGGATCGGATTCTCTAAATCG |
| 1223 | GCAGTAGAGTAGGTAGAGATTAGGCAAAGGCGATAAACATTAAATGTGAACAAACG |
| 1224 | GCAGTAGAGTAGGTAGAGATTAGGCAAGGAATAGAAAGGAACTTATTTTG |

TABLE 8
Staple sequences used for the T3 triangle 4 folding.

| SEQ ID | |
|---|---|
| NO: | |
| | Core structure oligo sequences (5′-3′) |
| 1225 | GTGAATAACCTTGCTTCTGTAAATCGTTTTTCGCTATTA |
| 1226 | CCGGGGGTAGGCGGATTGTTTTTATTGTTCTGAAACATGA |
| 1227 | ATATTATTGAGGCGTTTTAGCGAATAGATAAGTCCTGAAC |
| 1228 | AACGGGTAAAATCAGATTAACGTCAACAGTACGGTGAAGG |
| 1229 | TATAGAAGTGATTGCTGAGCAAAACAGAGCCAGCCCGGAA |
| 1230 | GAGCAGGTTTAACGTACGTATAATAGGAACCTCGGAACC |
| 1231 | TATCAGGGGGGGGAAAAGCACTAAGCGGGAGCAGACAGGA |
| 1232 | AATAGCAATAGCTATTTTTCTTACCGAAGCCCTTTTTAAG |
| 1233 | CCGATTTAGAACCTACCAGTTGGGCGGTTGTGTACATCG |
| 1234 | CGATGGCCTTTGGGGTCGAGGTGGGGGTGCCCCGGAAGC |
| 1235 | CCACCCTCTAGGTGTATCACCGTAATTAATTATGAAACAA |
| 1236 | CAAGTTACTATACAGTAAAAATGAAAACACAAGAATTGAGACAGCGGATCTCACGG |
| 1237 | AAGTATTAATTAGCGGTCGAGAGGGTTGATAGGCGGGCC |
| 1238 | TAGAAGAACTCAAACCTTTGATTAAATTAACCGTTGTAG |
| 1239 | GAGCTCGATGAATCGGAGAGAGTTTAAAGCCTCCGTGGTGG |
| 1240 | AATTGCGTGCACCGCTCACAATTCTGAGAAGCGTGCTTT |
| 1241 | TATTACTGGTAATATCATACCTACCCTCGTTAAGGGCGCG |
| 1242 | TTCCGAAAGGGCGAAAAACCGTCACATAAAAAAGGAGCG |
| 1243 | ATTCACAAACCACCAGAACCATTTTTCACCAGAGCCGC |
| 1244 | TTTAGCACAACGCCTCGGGAGAAACAACCCTCCAAATA |
| 1245 | GCCACGGGAACGGATAACCTCACCGGTTTTTAACAATCGGCG |
| 1246 | ATCCTGAATCATATTAAATCAAGTTTTTTTAGTTAATATATTT |
| 1247 | CTTTCAACAATTTACCGTTTTTTTCAGTAAGCGTCATACATGG |
| 1248 | TAATATCCGGTATTCTAAGAACGCTATCCCAAAGCTACAATTT |
| 1249 | TCAGACGATGCATTAAATTCGTAAATAAAGTGGCAGCAAGCGG |
| 1250 | CCTCCTTTTTCGACTTGCGGGGTTACAAATTTTTTAAACAGCC |
| 1251 | GCTCATTTAGAGAGAATGCGTAGATTTTCCCTACTATGGCTTCTGAAATAATCCT |
| 1252 | CTGCGCGCCTGTGCACGCTTTCCATGAAATTGTTATGAGAAGCCCAA |
| 1253 | TAACAGTGCCCGTATAGGTCAGTGCAGCCCTCATAGAGCCAGATGAA |
| 1254 | AGCGCCATGTTTACCAACGCAGAATTAAGCCCTTCTCCGT |
| 1255 | CCCTTAGAAAAGAACGCGATTTTTAAAACTTTTTCAAAAACAAA |
| 1256 | AATTTCATTTGAATTATAAGTATACCACCCTCAGACTCCT |
| 1257 | GGGATTTGTTAGTAAATGAATTTGCACGTAAAACAAGCTCAAACTTAAATTTCT |
| 1258 | ATTAATTTTGAACGCGCCTGTTTTTTTTTCAACAA |
| 1259 | CGCGCTTAATGCGCCGCTACGAATCAGAATCGGAACCATACGAG |
| 1260 | GTCACCTTTTTACTTGAGGTCCCGGAATTTGTGA |
| 1261 | AGAGCCGCAAGAGAAGGATTAGGAGAGGCTGATTTTCAGCGTAACAC |
| 1262 | CCCTCAGAACCGTCAGACTTTTTTTAGCGCG |
| 1263 | TTCTGCCATTCCTGTGGTCGGGAATGAGCTAACTCACATTCACCCAAA |
| 1264 | TTCATTTCAATCATTACCGCGGCTTTATTTTCATCGTAGGAACCAATC |
| 1265 | TAATGAGACCTGTCGTGCCAGCTCCAGCGGCCTGTTCTTCGCGTC |
| 1266 | AATTACCTTTGAATACTGAGTTTCTCCACAGACCTTGAGCAAATAA |
| 1267 | ATTACTGGTAATAAGTTTTAACGGAACAGTTACATGTACGGATAGC |
| 1268 | GAGCAAGAAACAATGAAAACGTACCGGAAATTTTTTTATTCATTAAA |
| 1269 | GATACAGGAGTGTTTGACGCTCAATCATCAATTAATGGAAGGGTGGCAA |
| 1270 | CATATCAAAATTATTGGATTATATTGCTTTG |
| 1271 | GTAACGAGATGAAATAAAGAAATTAACATAA |
| 1272 | GCGAATTAGTTTTCACGCTGCGGCCAGAATGCCCTTTTTTCATGTAGA |
| 1273 | AAGTGCCGGGTTTTGCTCAGTACCCAATACTTTATCGGCCTAATCAGT |
| 1274 | CTTTTACATGTAGCATGTCACCAGTACAAACTGCCACCCTGAAGATGA |
| 1275 | CAGAACAAACGGTACGGAGGCCACATAGCTGTGCACGCGTCAGTGTCA |
| 1276 | AAGGTAAATATTGAGAGATAGACTAATAATAAAGAAACGATTTTTTGT |
| 1277 | CATTTAACCAGTACATAAATCAATATATGTGAAAGAAAAAACATCAAG |
| 1278 | TGGATTATAACAGGAAAATTTTTCGCTCATGGAA |
| 1279 | GCAGCAAAGGTCAGTAAGAGCCGTTATTAGACTCAGCGGG |
| 1280 | CAATTCGACCATTGCGCCTGCAACACCTGA |
| 1281 | AAAGAGGATTGACAAGGATTTTAACGCCAAAGTAAATTGTAGACTGAGACGACG |
| 1282 | ACATCGCCGCAAAGAATACCAAAATGCTGTAGAGAGGTCAAAAAGAA |
| 1283 | TTTCGCACAGCCGGAATCCGCGACCTGCTCCACATCCCTT |
| 1284 | GAACCCTTTACATTGGCAGATTCATATTCACCAGAAGGAGCGGAACGCGGTC |
| 1285 | ACATAAAAGGGACATTAATGCGCGTACCGAACGTGGCACAGACAATATTTT |
| 1286 | TCAGATGCCGGGTTACCTTTTTTCAGCCAGCGGTGCCGGTGCCCCCTGCA |
| 1287 | TGCCAACGGCAGCACCTGCCGGACTATCATTTTGCGAACCACCACACCCG |
| 1288 | ATCATACAGACCATTATCTGCGAAGCTTGCCC |
| 1289 | TGTCGAAACGAGGCGCAGACGGTCTGAACCTTTTAGTTT |
| 1290 | GTGTAGCGGTTTTTCACGCTGCGGCCTCCGGCCAGAGGG |
| 1291 | TGAATGGCTATTAGTCTTTCTGGCCAAAAGAATACTTAGAAGCAATAGA |
| 1292 | GCTCATCGCTAATACATCAAATATCCTAAAGCATCACCTTGGGCGCGAG |
| 1293 | CTATATTTTCATTTGGCAATCATAAACGTAACAAAGCT |
| 1294 | CTTATAAATCGCTGATTGCCCTTTTTTTACCGCCTGGCCCTG |
| 1295 | AATCAACAGTTGAAAGGAATTTTTTGAGGACGTGAGCC |
| 1296 | TAACAACTAGTTGGCACCACGCTGAGAGCCA |
| 1297 | ACCCTGACTATTATAGCACCGCTTCGTTTTAA |
| 1298 | CGTCAGCGTGGTGCTGGTCTGTGCTTTTTGCGCGCATCGTATAGGTCA |
| 1299 | CCACGCTGGTTTGAACCAGCTTACGGCTGGAGGTGGTTGCGG |
| 1300 | ACACTGGTCTTTGCTCGTCATAAAATCTGGTCAATAGATT |
| 1301 | TGTTACTTTCAATCCGCCGGGCGCGTCCAGCATTTACAAAGAACGTTA |
| 1302 | TCATGGTCCGAGTAAAAGAGTTTATCTTTTTTAAATATCTTT |
| 1303 | TATTACTTTTTGCCAGCAACTCGTAGGAGCACTATGAGCCAAAGGTTT |
| 1304 | GAACCACCAGCAGAAGATTAAAAAAACTGATAGCCCTAAA |
| 1305 | CCAACGCGCGGGGAGAGTTTTTCGGTTTGCGTATTGG |
| 1306 | AAAATCCTGTTTGATAAGCCGGCGAACGTGGC |
| 1307 | CCGCACAAACGTGGACTTTTTCCAACGTCAAATCGGCAAAATC |
| 1308 | CGATTAATTTTTGGGATTTTTAAACATTTTTGAGGCATTAAATC |
| 1309 | CAGGAAGAAGGGGACGACGACAGTTAGATGGGGATGGCTTACACGACC |
| 1310 | AACCGGATAACGAGTAAGGAATTACGTTTACCGATAGCGT |
| 1311 | AGTAACATTTGTAGAACGTTTTTTATTGCCGTTCCGGCAAATCGGCCT |
| 1312 | AGAGGGAAATAAAAACGTTTTAAAACAGTTGATTCCCAAT |
| 1313 | CTGGTGCCGGAAATCTTTCCAATACTGCGGAATCGT |
| 1314 | GATACATTCCCAAATCAGGGAACCTTTGTATCATCGCCTGATAAATTG |
| 1315 | GGTACCTCCTCACATTTTTTTGAGGAGCGGCTGG |
| 1316 | CAGATGAAATACCAAGCGCGAAACAAAGTACCCTTATGC |
| 1317 | TTAGTGATGAAGGGTAAAGTTAAACGATGCTGCGTCTCGT |
| 1318 | CTGATTATCAGATGTTTTTTGGCAATTCGTCTGAA |
| 1319 | TTAACACCTCTGACCTGAAAGCGTCAGAGATATTAATTTTAAAAGCT |
| 1320 | TCAACTAATGCAGATACATAAGAACTGGCTCATTATCAGCGATT |
| 1321 | CAACTTTGCGGATAACCTGTTTAGGTGGCATCA |
| 1322 | GGCAAGATAAAACAGAGGTGAGGCTGAAAAATAAACCCTCAATCAAT |
| 1323 | AGGCTGTCCATCACGCAGTAATAACATTTTTCACTTGGTG |
| 1324 | CGCAAGAAGTCATTGCAGGTCAGTGAATAAGCGAGTAGAGAATATAA |
| 1325 | TGCCATCCCACGCCCCAGCCCGAGGAGTCCACTATTAAAG |
| 1326 | CGAGAGGCTTTGTCAGCAGCAACTAATTGCTTTGAGGAT |
| 1327 | GTGTTCAGCAAATCGTTAACGGCAGCGCCAGGGTTGCCCTTCCCCG |
| 1328 | GGCGGCCTAAGAATTTTTAGCGAAAATCCCGTAAAAAAA |
| 1329 | CGCTATTACGCCAGCCGACAATGACGCCATCCAGCTCATAGCCTCA |
| 1330 | TAACTATAAGACGCTGAGAAGAGTCAATAGTGCCGACAAAAATAAGG |
| 1331 | TCCTCATTAAAGCCCAGACGATCGGTCATAATCAAAATTACCGACC |
| 1332 | GCAAAGCGGATTGCATCTTTTTAAAAGAACTATCATATTTTTCCCT |
| 1333 | ATAAATAAAATCTTTTTAAAAATCAGGCCAGGCA |
| 1334 | TCTAGCTGATGTAGTAGCTTTTTTTAACATCCAATAACTGAAAAG |
| 1335 | TAAATTTTCCGCTTTTATACCGATAGTTGCGCTGGCGAAA |
| 1336 | TACCATTATACCAGCGCCAAAAAACGGCCAGTGCCAAGCTTTCAGAG |
| 1337 | TTAGTATCTTACCAACGCTAATTTGACATTCAACCGATTGAGGGAGG |
| 1338 | CAAAAGGGTTCGCATTAAACGTCTCAGCAGCTTTTTAAAAAATCAT |
| 1339 | TTCGCGTCTGGCCAAACAGCTTGGCGGGATCGTCAATAAGAATAAA |
| 1340 | AGAGAATCGCAAATATTCAACCGTTGTGTAGGTAAAGATTGCAATGCC |
| 1341 | GACGACAAATTTAATGAAGCCTGTTTTTCATCATCAAGTTTGCC |
| 1342 | AGAGCCTAATTTGCCAAGGTTTTGAAGCCTGCATGTTCAGCTAATGCA |
| 1343 | CGGTGTTTTTACAGACCAGGGCAAAAGAATACACTAAAACACTC |
| 1344 | AGGAACAAGGAAACGTATAAAAATTTTTACGCAG |
| 1345 | AAGCAAACTCCAACGATCGGTGCGGGCCCCAACTGTT |
| 1346 | GGGAAGGGTAAGTTGGGTAACGCCTGTGCTGC |
| 1347 | AAACGAACGGCTTTGACCGATATAAGTTTCTTTTTTTCCATATA |
| 1348 | CGTTAACCTAATATTTTGCCTGAGGATCTACAAAGGCTATCGCCACCC |
| 1349 | GTTAAAGGTGTTAAATAAAAATAATGAGTAAGAAGCCT |
| 1350 | CGCCAGCATCAGAACCCCGCCTCCCTCAGAGCGGGTTATAGTGTGATA |
| 1351 | TCTTTGACCCCACCAGTCATTTTTGACGTTGGGAAGAGAATACCACA |
| 1352 | TTCTACTAATAAAAGTAATCGTAAAACTAG |
| 1353 | TCGCAAATGGTCATTGATAATTTTTCAGAAAAGCCCCAAAAA |
| 1354 | ATCGGTTGTTAGCAAAATTTTTTTAGCAATAATTTTTAACCAGGAAGA |
| 1355 | CTCAGGTTTTTGGTTTAGTACCGCAATCCAAT |
| 1356 | AGTTAATTTCATCTTCCGCAAGACATCCTTGAAAACATAGCGATAGCT |
| 1357 | CCGAACAAAAAAGGGCTGTCACAAGCGACAGAGGCATTTTTGGCCTTGA |
| 1358 | TTATTTCACGTGGGAACGAGTAACAACCCGTCGCCTTT |
| 1359 | CGAGGCATTTTTTAGTAAGATTTAGAAAATCTACGTTAATACGAAAGAG |
| 1360 | AGTAATTATGACCCTGTAATATTTTTTTTTGCGGGAGAGCATAAAGCTA |
| 1361 | TGTCTGGATTCGGTCGCATCGCCCACGCATAATTAAGAGGAAGCCTCTT |
| 1362 | AGCGCCATTCGCCATTCAAACGAGAATGACCATTTCATTGA |
| 1363 | AATTGTAGGAAGGGTTTTCCCAGTCACGACGTTGAACGCAATTTGCTT |
| 1364 | TCTCTGAGTTTCAGATTGCGAATAATAATT |
| 1365 | ATCCCCCTATCAGTTGAGAGCAACCTGGCTGACCTTCATTTTTTAAGAGTAATC |
| 1366 | TCGAGGGTTGCAAGGCCCTAAAGGACGGAGTGA |
| 1367 | TGACCTAATAAACAACGTTATACAAATTCTTATTACGAGC |
| 1368 | TGAAACCATGTTAAAATGAGAAAGGCCGGTTGAGGCAGGTAGAATGGAAGCACCGT |
| 1369 | TTAATGCCGGAGAGGGTAGCTATTATGATATTTAAATTG |
| 1370 | ATTAGCGTTCGATAGCAAGGAACCCTCATATATTTTAAAT |
| 1371 | AGCTTTCATCAACGTCAGGATTAGCAATAGGAACAACAAC |
| 1372 | TATGCATTTTTCTAAAGTACGGAGAGTACCTTTTTTTAATTGCTCC |
| 1373 | CGCATAGGGGACTAAACATGTCAATCATTTTTTTGTACCC |
| 1374 | CGGATTGATTTTTCGTAATGGGAACCGTGCATCGCGAACCAGACCG |
| 1375 | TTGCCATCAGGAGACAGTCAAATCACCATCAATTTTGAGA |
| 1376 | CTGATGCACACGCCACCACCGGAAGCCACCCTCAGAGCC |
| 1377 | GTTTGAAACACCGGAACCAGACCTCAGAACCGCC |
| 1378 | TCTAAAGTTTTGTTTTGGGAATTAGATTTTTCCAGCAAAA |
| 1379 | TTTTCACCTCACCAGTAGCACCATAATCAGTATCAATAGA |
| 1380 | TAGCAACGCATGAGGACACTACGAAGGCACCAACCTAAAA |
| 1381 | AGTCTGGAGCAAACAGAGACTACCGAAAGACAGCATCGG |
| 1382 | AGGTCTGAGTAAAATACGTAATGCAGTTTCCATTAAACGG |
| 1383 | CTGAGGCTTGCAGGGAAACGAGGGTTGTATAAGATGAACG |
| 1384 | GTCTTTCCAAAAGTAAGCAGATAGGTGGAGCCGGTGAATT |
| 1385 | TTGACAGGTTTTCATAGCCCCCTTCACCGGAAAAATTCAT |
| 1386 | GCTATTTTGCATAACGGATTCGCCGCTTATCCCATCCTAAGCTGTT |
| 1387 | TTTATCATTCCAAGAATAATCGTTTACGAGAATGGAAA |
| 1388 | AAAATCGCTGCCCCAATAGCAAGCTTAAACCATAATATCAT |
| 1389 | AGATAACCCAGGGAAGCGCATTAGACGGAGGGTAAGCCTTTAC |
| 1390 | TCAAGCCGTTTCACTGCCCTCTGTGGTGGTCATA |
| 1391 | AAATAGCATTGAGCGCAGTACCGCACTCATCGAGT |
| 1392 | TCTGAACAAAGTCAGGAGAATTTGAACCATGCCGCCAG |
| 1393 | CCAGAATCCCACACAACCTAAAGGTCAAGTTTCACTACGAACTGT |
| 1394 | TTTTAATCATTGTGAATTAAACGGAGAGAACTGAC |
| 1395 | TTTTGATACTCAACATCAAAATAGTGACGAGAATGGTTTAATT |
| 1396 | TAAAATGTTGGCTTGAGAACACCAGATTCATTA |
| 1397 | CAGTTTGTCGCACTCCAGCCAGCTTTCCGGTCAGGGTT |
| 1398 | TAATGGGTGGGTCACTGTGGTTTTTCT |
| 1399 | TCCAGTGAGACGGGCAACAAAAAGAATCAGCAGGC |
| 1400 | AGAAAGGAAGCACATCCTCATATGGAACAAATAGGGTTGAGTGT |
| 1401 | TCAGTTACGGAACGGTCGGTGGCTTTGCCC |
| 1402 | TTAAACAGTAGGGCTTAGTCCAGACTAGATTATGTAAATG |
| 1403 | AGTTACCACACCACGCAACATATCATGTAATAAGCCAACGCTCAGAAT |
| 1404 | TGTTTGGGAATAAGTCCAGTATAACTAGAAA |
| 1405 | CGCAAAGAGAAGGAAGAAAATACATACATAAAGGTACT |
| 1406 | TCATAATTTTAGGCATAATTCTATTGAGAATCGCCATATTTCAGT |
| 1407 | TAGCAACTTCGAGCCAGACTCCTATGTGAATTTCTTAA |
| 1408 | AGGTAAAGGAGGCATTAACGCCAAAAAAGAAATATTACGCAGTATATTT |
| 1409 | TCTTGTTAGCAAACGTAACCGAGGATAAAACGAAAGGCTCCAAAAGGA |
| 1410 | TATAAAGTAAATTTATCCCTCCGGCTTAGGTTCAGGTCAT |
| 1411 | TGAGCCAAAAGAACTGGCATGATTAAGTAATAAGAGTTCT |
| 1412 | TGTAGCCGGGTCGGTTTATCAGCAATAACGGAAT |
| 1413 | GACTTTTTGCTACAGATAACGGAACAT |
| 1414 | TTGTTTATTACAGGTAGAAAGATTCCAAATGCTTTATAGT |
| 1415 | TAGTTCAGAAGGCTGCGGAAAGACTTCATTTTTATATCG |
| | H1 design oligo sequences (5′-3′) |
| 1416 | GCAGTAGAGTAGGTAGAGATTAGGCAGATTGTTTTTCTGTATCTTTTGATACGTTAGC |
| 1417 | GCAGTAGAGTAGGTAGAGATTAGGCAAAAAAGAGCCACGTCTTTCCAGACTGCTAAACA |
| 1418 | GCAGTAGAGTAGGTAGAGATTAGGCAGGCGCTAGGGCGCTTAGAGCTTGA |
| 1419 | GCAGTAGAGTAGGTAGAGATTAGGCACGTTGGTGATTATCATCACCAGTCAGAGTTTG |
| 1420 | GCAGTAGAGTAGGTAGAGATTAGGCACGCTGGCACGTGAACAAAGAAACC |
| 1421 | GCAGTAGAGTAGGTAGAGATTAGGCATTCGAGCTTCAAATGCGTTTTGCC |
| 1422 | GCAGTAGAGTAGGTAGAGATTAGGCAGAATAGAAGGATTCTCACGCAAGGCACCTTCC |
| 1423 | GCAGTAGAGTAGGTAGAGATTAGGCAAAGGCGATCAGATTAAATGTGAGCAAACGG |
| 1424 | GCAGTAGAGTAGGTAGAGATTAGGCAATCACGTTGAAAATCTCCAAAGAC |

TABLE 9
Staple sequences used for the T3 triangle 5 folding.

| SEQ ID | |
|---|---|
| NO: | |
| | Core structure oligo sequences (5′-3′) |
| 1425 | TACCGCTGGTAATATCATACCTACCCTCGTTAAGGGCGCG |
| 1426 | GCGAAAGGGGGATGTGCAGTGCCATAGCAGCCCCAGGGTT |
| 1427 | CGTCCGTGTCATTAATTGCGTTGCCGGGTGATGCCGGGTTACCTG |
| 1428 | CAGGGAAGCGCATTTTTTTGACGGGAGAATTAACTGAACA |
| 1429 | CGCGCTTAATGCGCCGCTACGAATCAGAATCGGAACCATACCGG |
| 1430 | AATCGTCGTGATGCAAATCTTTTTAATCGCAAGACAAAGATGAT |
| 1431 | CGATGGCCTTTGGGGTCGAGGTGGTTCTTCGGGGTTTCT |
| 1432 | CCAGAATCTTCACGGTCCTAAAGGTCAAGTTTCACTACG |
| 1433 | AGTACATAAATCAATATATGTGAGTGTTTTTATAACCTT |
| 1434 | TGAACCATATAACCTCTAATGGAAGGGTTAGATCAATATTTGCTTTG |
| 1435 | GAGAATAACATAAAAAGCCAGCTGATAAAATTTTTGAAACGCAAAGA |
| 1436 | GCAAGTTTTTCCGTTTTTATTGAGGTTTTTTTTTAAGCCTTAA |
| 1437 | CACCCAAAGAGATAAAGAAATTGACGTATAATTGATATATCTGAATT |
| 1438 | TGGTGTGTTCAGCAAGAGTAACATGTTTTTATTGTTCCAGTAAGCGT |
| 1439 | CCCTGCGGCTGGTAATCGAATTCGTGCGGCCAGAATATACGAGAGGG |
| 1440 | AGAGCCACCACCCAATAGAAAATTCATTTTTATGGTTTAC |
| 1441 | AAATAATAATTAAACCAAGTACCGTAGTTGCTATATAGAAGGC |
| 1442 | CACCCTCCACAAAATTATTTGCATTTGTTTACCCAATCCAAAATAAA |
| 1443 | CGCAGAGGATAACGGAAGGTGTATTCAGAACCACAAACAACGCCACC |
| 1444 | CATACATGAAGTTTTAATAAACAGTTAATGCTAAACATC |
| 1445 | CCGATTTACTTCTGAAACCGGAAACAATCGGCGAAACGT |
| 1446 | ATTTCATTAAACAAAATTAATTACCCCCTGCCAGTACCAGATGATACA |
| 1447 | TCCGAAAGGGCGAAAAACCGTCACAGCGCCAAGGAGCG |
| 1448 | ACTTGATTTAGTACTACAGTAACAGTACAGATTTTGCA |
| 1449 | AAAGGTGGCAAAAGTTGGGTAACGTTTACAGACCCAGCTACAATTTTA |
| 1450 | AGCAAGCAAATCCTTTTACATCGGGAACGGGTTCCCATCCTAATTTAC |
| 1451 | CGGTATGAGCGCTCACGTGTCACTGCCAGCACGCAGCAAGCGG |
| 1452 | GTGCCCGTACGGGGTCAGTGCCTTCAATACTTTATCGGCCTAATCAGT |
| 1453 | CAGAACAAACGGTACGGAGGCCACGTGCACTCCGGCATCACACTGTTG |
| 1454 | GCTTCTGTAGCAGAACGCGCCTGTTTTTTTATCAA |
| 1455 | TTCGTCACCCGCCACCAGATTTTTCCACCACCAGAGCCGCCGC |
| 1456 | TATCCGGTATCTATCATTACCGCTTTTTCCCAATAAGAACGCG |
| 1457 | ATCAAGATCACTCATCGAGAACAACAATAGATAAGTCCTG |
| 1458 | AGGATTAGGATGCCATTTGTTTTTGAATTAG |
| 1459 | CATTCCAAGAGAAACACGAATTATTTTTGCTCTATTTCGG |
| 1460 | TTTATTTTTTTTGTCACACTGCAAGGCGATTCAT |
| 1461 | CCAAGTTACCTTACACGTTTCTTTGCTCGTCAATTTAACAGAGCATGT |
| 1462 | GATGAATACGCCACCCCACCGTACTCAGGAGGTAGCGGGGTCATTTCA |
| 1463 | CGGAAGCATAAAGTGTAATTGTTAACCGAGCTGGGTAAAG |
| 1464 | TTGGGAAGGGCGATCGGTGCGGGCCTTTTTTTTCGCTATTAC |
| 1465 | TATCAGGGGGGGGAAAAGCACTAAGCGGGAGCAGACAGGA |
| 1466 | CCCTCAGAGCTAGCCCCCTTATTTTTTTGCGTTTGCCATC |
| 1467 | TAGAAGAACTCAAACCTTTGATTAAATTAACCGTTGTAGATCGTTAA |
| 1468 | CATCAAGATGAATTACCTTTTTTAATGGAAACAACAAGAAAGCAAAAG |
| 1469 | CGGCATTTAACCTATTATTCTGAAGAAACAAAATTACCTG |
| 1470 | CCAGCGCATGCCCGCTAGAGAGTTGCGTGCCTCCGTGGTGGT |
| 1471 | GTACCGTATAGCAAGCCCAATAGAACCTACCATATCGACGAGGTGGAGCCGCCAC |
| 1472 | ACAGGAGGTTGAGTTGACGCTCAACTGATTATTTGTTTGGATTTGGCAA |
| 1473 | GGGAACGGAACGATTTCGTAAAACAGAACCCTACTATGGAATCCTGACAGATGAT |
| 1474 | TCGGTCAGGAGTGTACTGGTAATGCTTTTGGCGGATAACCCGGAATTTCGCCTG |
| 1475 | CGTGCTTTATTGCAGGTCAGACGATTGGCCTTAGCCAGAAAGTATAGGTGCCGT |
| 1476 | ATAAATCCTCATTAAGATATTCGCCACCCTCAGAAGCCGTAGATTATTGCTT |
| 1477 | CTTATAAATCGCTGATTGCCCTTTTTTTACCGCCTGGCCCTG |
| 1478 | TAGAAGTACCATTGCTGAGGCGGTCCCTGA |
| 1479 | GACGATGGTGCCGGTTTTTGCCCCCTCCGCAAGAATGCCAAC |
| 1480 | TCTAAAATAACCCTCAATTAACACCGCCTGC |
| 1481 | GGGGTCATTGCAGGCGCTTTTTTTCGCACTCAATCCGCCGGGCGCGGTTG |
| 1482 | GAACCGATAATAGATTAAAAGCATCGAGCCAGCAGCAAATGAACATGTT |
| 1483 | GTGTAGCGGTTTTTCACGCTGCGTTGCCGCCAGCAGGGG |
| 1484 | AACAGTGCGAAGATAAGAGCACTAAATACATTCCGGCCAG |
| 1485 | TATAATGCTGTAGCTCCTCATCTTGGTCAATCATAAGG |
| 1486 | CGGCAAACGCGGTCCCAGCCCGAGGAGTCCACTATTAAAG |
| 1487 | TGATTCCCAAGAGGTCGTACCTTTTTGAAAGA |
| 1488 | AACCTGTCCATCACGCAGTAATAACATTTTTCACTTGAGT |
| 1489 | CTATTATACAAATATCCCAAAATAGGACAGATGAACGGTGGGAACGAG |
| 1490 | GTCTGGTCCGGCCAACAGTGAGACGGGCAACAAAAAGAAT |
| 1491 | CATCAACATCTGGCCTTCCTGTAGTTTTAACCATTGCATCACACGACC |
| 1492 | GAACCCTTTACATTGGCAGATTCCAGAAGCATTTTGCGGAACAAAAACAGCG |
| 1493 | TGGATTATAACAGGAAAATTTTTCGCTCATGGAA |
| 1494 | ATCGCCATTAAAATCTCTGATAGCGGCTATTAGTCTTTAA |
| 1495 | AACGAAAGTGCCGGACTTGTAGAAGGCAGCCTTGAGGATTACTCGTAT |
| 1496 | GAGCGGAATTATCATTTTTCATATTCTCGTCTGAA |
| 1497 | CGTTATTAAATCCCGTGATCAAACGGAAAAAGAGACGCAGCCAGCTTT |
| 1498 | TATTACTTTTTGCCAGTTAGACTTGAAGGTTATGGTGCTGGGCAGCAC |
| 1499 | TCTCCGTGGGAACATAACACGTTAATAAAACGAACT |
| 1500 | ATTCGCGTTAAATGTGAGCGAGTAACAACCAGGCATA |
| 1501 | TTCCAGTCGGGAAACCTTTTTTTCGTGCCAGCTGCAT |
| 1502 | AAAAATCTGCCAAAAGGAATTACGCGTCGGATATTCATTG |
| 1503 | AGAGGCGGCCAGCTTACGTCGGTGGTGCCATCAAATATCAATCTTTAG |
| 1504 | AAAAAAAGCCGCACAGGCGGCTTGCAAAGCGGAATAGGAAGTTAAATC |
| 1505 | AGCGATTATACCAAGCATTCTTAATTGCTGAAGCAACTAAA |
| 1506 | GTTAAACGATGCTGATCATAAAAAATTTTAAAAGTTAACCACCACACCCG |
| 1507 | ATTTTTGCGCGCAGACTGACCCCCCATTAAACGGGTAAAATACGTAAT |
| 1508 | TGCGCGAAGCGAACGAGTTTAGCTAAAGACTTGTCAGAAGCAAAAGA |
| 1509 | ATATAAAAGGGACATTTTTTGAATCCTAAAACGTGGCACA |
| 1510 | AATTTGTAACGTGGACTTTTTCCAACGTCAAATCGGCAAAATC |
| 1511 | CCACGCTGGTTTGCCGTTTTTTCGTCTTTTCACCGCGCGGGG |
| 1512 | ATCAATATCTGGTCAGTTGGTTTTTAAATCCAGCCAGC |
| 1513 | AAGTACAAAGGACTAAAGACTTTTTCATGAGGAAGTTTC |
| 1514 | AACAGAGGTCTGACCTGAAAGCGTCAGAGATATAAATCCTTTGCAAG |
| 1515 | GATAAAAACAGGACGTTGGGAAGGGCTCATTGTAATAGT |
| 1516 | CAGAGGGGATACCAGTGCGTTTTAAGGTCAGGATTAGAGA |
| 1517 | AGGGTAAAAGCACATCCTCAACTGACCAACTAATTGCTCGAAGCCCG |
| 1518 | GCGCGCCTCGAGTAAAAGAGTAGTTGATTTTTAGGAATTGAG |
| 1519 | AATATACCGAACGAACCACCAGCACACGCTGAACCTTGCTGAACCTC |
| 1520 | GCCACTACGAATACACTAAAACAAAAAATCTCTTTTGAT |
| 1521 | ACGAGTAGTAAATTGGGCTTGATCAAGAGTAATCTTAGGCTTTG |
| 1522 | CGATTAATTTTTGGGATTTTTAAACATTTTTGAGGCTACAAACA |
| 1523 | GAGAGATAAAGAATTTTTAGCGAATGTTTACCAGTCCCG |
| 1524 | GACAATATCTGGCCAAAAGAATACAATAGATACAACTAACGGAACG |
| 1525 | CAGCAGGCTTTTTAAAATCCTGTTTGATAAGCCGGCGAACGTGGC |
| 1526 | CGGCTGGAGGTGTCCAGCATCAGCTAATGAATAGCAGCAAGCATCA |
| 1527 | GACTTTCTCCGTGGTGAAGGGATAGCTCTCACTTAAATTT |
| 1528 | TACAGGTAAGGCTTGCTGTGAATTAAATTGTGTCGAAATTTTTTCGCGACCTGC |
| 1529 | TAATGTGTAGGTAAAATTTATCATTCTTAAACAGCTTGA |
| 1530 | AGCCCCAAAAACACCATAAATCAACGTAAAACAATAATAA |
| 1531 | TTTATTTCAACGCAAGGATAAAAGACCCTGTGAGATCTA |
| 1532 | TTAGCGACCCTGAACAAAGTCAGCGCAACTGCACCACGG |
| 1533 | AAGACAAACATTAAAGCCCAAAAGTTAAATAATTATACA |
| 1534 | TACGGTGTCTGGCCAAAGGCCGGAGACAGTCGCATAAC |
| 1535 | AAAATGTTTTTTTTGACTGGACCTTATGCTTTTTATTT |
| 1536 | CAGTACAACTCATAGTTAGCGTAACGATCAAGCAGCGCCA |
| 1537 | AGACTCCTTCTTTCCAAGCGCCATTCGCCATTCAGGCTG |
| 1538 | TGGAGCAATCGGTTTAAAAGGAACAACTAAAGCGACGACA |
| 1539 | TAATTGTAACAAGAGACAATCATAATTAGCAGTGGCAT |
| 1540 | CAATTCTATTAATATTAAGCAAATATTTAAATATTTTC |
| 1541 | AATAGTGAGCAGGGAGTTAAAGGCTCGGTCGCTGAGGCTT |
| 1542 | CAGAAAACGAGAAGCATCGTAACCGTTACGTTGGTGT |
| 1543 | CATTCCACGATTGAGGAGTAGCATTAACACACCCTCAGAG |
| 1544 | ACGGAACAACTATTTTTATGCAGATACAAACGGC |
| 1545 | GCCGGAAACACCGGTTGTACCAAAAACATTATATTTTTAG |
| 1546 | CGCCTGATATCGCCCACAAATCACCATCTTTTTATATGAT |
| 1547 | CATCTTCTATCGATAGCAGCAGACTCCTCAAGAG |
| 1548 | CTCCAAAAGGAGCCTTTACCGATAGGGTAGCTAGGGTGAG |
| 1549 | CAACAGTTAATTCAACCAGACAGCCACTACAAC |
| 1550 | TTAGCAAGATTGACGGCACTCCAATAAATCATACAGGCAA |
| 1551 | AGATGGGCACTCCAGCCAGCTTTCGCCTCAGG |
| 1552 | GGTTATATTGACCGTAATCAGTAGTGTAGCGCGTTTTCA |
| 1553 | ATCAAAATCGTCACCACATTACCAATAAGGCGAACTGGCA |
| 1554 | ATATTTTACAATAAACAAAGCCTGTTTAGTATATAGGCGTT |
| 1555 | TACCGTCGTATCAGGTTGCCTGAGAACCCTCATATATTTTGCCTTTAG |
| 1556 | TGCGGGAGAAGAAGTTTCTTTTTTTCCATATAACAGTTTAAATAT |
| 1557 | AGTAATTTTCATTTGGGGCGCTTTTTAGCTGAAAAGTTTCGCAAATGGT |
| 1558 | ACATGATTTTTAGTATTAAGAGGCAACTATAT |
| 1559 | GATTCAAAATTTTTGAAATACTTTAAATTAAGCAATAAAGGGCAAAGA |
| 1560 | TTTTCATACGTCAGACCGACAGAATCAAGTTTCTTTTTAATTTAATGG |
| 1561 | GATTGACCGTAATGGGATAGGAATACCACATTCAACATTAT |
| 1562 | GAAAACTTTTTCAAATGTAAATGCCTATTAATTAATTTTCCCTTAGAA |
| 1563 | AATAACCTGTAGATTTAGTTTTTTTGACCATTACGGTAATGCCGGAGA |
| 1564 | AACTTTATTTTTATCATCCTGACGACCCAAATCAACGTAACAGCAGCGA |
| 1565 | ACCTCCCGACTTGCGGTTCATCGTAGGAAGAAAACATGTTCAGCTAAT |
| 1566 | TCAGAAAGTAGATTTTGCTAAACAACTTT |
| 1567 | CATAATTATCTAAGAACGCGAATAAGCAAACGTAGAAAATACATACA |
| 1568 | CCTCAGAGGAGAGTCCAAAGGCAGGTGAATAAATCATAGAAGAGTC |
| 1569 | CATAAAGCTAAATCGGAACCAGAGCTCAGAGCAAATTATTAGGGCGAC |
| 1570 | GGATGGCTTAGAGCAACCGTTTTTTCTAGCTGATAAATTAAT |
| 1571 | GACAATGACAACAACCATCTCCAAGGAAGCTTTTTAACTCCAAC |
| 1572 | TGTACCCCGGTTGGAGAGAATAGTCAGCTTGCTTACCGTGTGATAA |
| 1573 | AACGGCTACAGGACAAGAATTTTTCGGATATTCATTAGAAACACCAG |
| 1574 | GCCAGTTTGAGGGGAGAATTGCGTAGCATGTATCGATGAAGATACA |
| 1575 | AATTCGCATTTTTTAAATTTTTCGCCATCAAAATGCTTTAAACAGT |
| 1576 | GACGACGAGTTAATTTACCGGAATAGCCAGCATCACCGTCACCG |
| 1577 | ACCAGACCAAAAAAGGTTTTTTCACGTTGAAAATAGCGTCCAAGCATCT |
| 1578 | CGGAGTTTTTTTTGTATCATAAGACAGCATCGGAACGAGGGTAG |
| 1579 | ATTCGATTTTTCTTCAAAGCGAAAATCAGGTTTTTCTTTACCCTGA |
| 1580 | AGGGTAATAGTATGTTACGGAATAGTGAATTAAAATCACCGGAACCGCC |
| 1581 | CCTCCGGCAAACATAGCGATAGCTTAGATTAACTTAATTGTTTGAAA |
| 1582 | TCAGAACCGCCACCCCACCACCAGTAGCACATGAAACCGACCTAAA |
| 1583 | CAAAATCGAGAAACCAATCAAGGTTCCGCTCACAATTCCACACTAAT |
| 1584 | TGAGAACTTCCTTATTCCTGAATTAACGTCATTCCCAGT |
| 1585 | CCGTAATCGGCTGTCTATACGAGCCAGCCATACTTACCAACGCTAGTTT |
| 1586 | TAGCGTCTTTCAATGAAAAAGCTTTCAGTTGTAAA |
| 1587 | TGGGTAACTCACATAGCTGTTTCCTGTGTGAAAAGCCTGTCACAGTT |
| 1588 | TGTGGTGCTAATCATGGAGCCTCCGGGTGCCTAATGAGTGAGCGAGT |
| 1589 | TAGCTACAAATAAGAGAGGATCCTGAGCGGCGGGCCGTTCTGAGAAG |
| 1590 | TTCAGGTTTTATTTATACGTCAAACAGAGCCTAATTTGCCAGTCCGT |
| 1591 | TCTGGCACTTAGCCTACAGACCTTAATTTCTAAGAACT |
| 1592 | GCGAAACATCCATGTTTGACCTTCAGATGGTAGGCGT |
| 1593 | TTTTACCTTCTTTAGTGATGAATTAAGAGGAGCCGTC |
| 1594 | GCGAGAGGCAGACGACAACACTATCATAAT |
| 1595 | AGGCAAAAGAAGGCACCAACCTAACCACGCAATTTGCGTAT |
| 1596 | TGCCAGGGTGGTTTTTCTCGTCGCTCGTCAGCG |
| 1597 | AGAAAGGAATTGGGCGGTTGTGTGGAACAAATAGGGTTGAGTGT |
| 1598 | TCAGTTTACATCGATGCCGTTCATTCGACA |
| 1599 | TTTTTGAGCAAGAAACAATTTTTAAGCCAGGCAAGACGTTAGTAAATGA |
| 1600 | TGTATGGTCGCGGCACCGCTTCTGGTGCCGGAAAAAAAGTAAGCATTTTT |
| 1601 | TTTTTCAGTTGAGATTTAGGTCACTGCGGAATCGTTTTTTATAAAT |
| 1602 | TTTTTTTAACAACGCCAACCAGTATAAAGCCAACGCTTTTTT |
| 1603 | ATCTTACCAACCGAGCACAAGAATTAGGCAGAGGCATTTTCGAGTTTTT |
| 1604 | AAGTAATAATATAAAGTACCGACAGAATCGCCATATTTTTT |
| 1605 | TTTTTCAACAGTAGGGGACGCTGAGGTCTGAGAGACTACAAATGCAA |
| 1606 | CAGAAGGAGAAGCCCTGAAATAGCAATAGCTTGAGCGCTAATATTTTTT |
| 1607 | TTTTTCAGAGAGATAACCGAAACGCACATATGCGGAATAAAC |
| 1608 | TTTTTTTCAGTGAATAGAAAGATTCATTTTTT |
| 1609 | AAAAGGTAAATTCTTACATGTAATTTGAGTTAAGCCCAATAATAATTTTT |
| 1610 | TTTTTCCAGTAATAAGAGTCTGTCCATCCTTGATTAGGTTG |
| 1611 | TTTTTGATAGCCGAACAAAGTTACTGATTTCAGCGGAGTAGGTAAATCATTGCCT |
| 1612 | GTTGCGCCCGATATATCGCTTTTGCGGGATCGTCACCCTCAAAGCTGCTCATTTTT |
| | H1 design oligo sequences (5′-3′) |
| 1613 | GCAGTAGAGTAGGTAGAGATTAGGCAGGCAATTCGAACCCATCAGCATTGACGACCGC |
| 1614 | GCAGTAGAGTAGGTAGAGATTAGGCAACGACGGCATCTCATTTTCAGGGAACACTGAG |
| 1615 | GCAGTAGAGTAGGTAGAGATTAGGCAGGCGCTAGGGCGCATAGAGCTTGA |
| 1616 | GCAGTAGAGTAGGTAGAGATTAGGCAAGCTCATTAGAAACCACACCAGTCAAACCGAA |
| 1617 | GCAGTAGAGTAGGTAGAGATTAGGCACTGCTCATCGTTGAGTAACATTAT |
| 1618 | GCAGTAGAGTAGGTAGAGATTAGGCAAATCCCCCTCAAAATAAGTTTTGCGTAAGAGC |
| 1619 | GCAGTAGAGTAGGTAGAGATTAGGCAGCCTGTAGTGTAAACGCTAATAGTGAGGATAA |
| 1620 | GCAGTAGAGTAGGTAGAGATTAGGCAAAGATCGCTGAGGAAGATTGTATTTGTTAA |
| 1621 | GCAGTAGAGTAGGTAGAGATTAGGCAAATTAAAGTTTTGTCGTATTACGC |

TABLE 10
Staple sequences used for the T3 triangle 6 folding.

| SEQ ID | |
|---|---|
| NO: | |
| | Core structure oligo sequences (5′-3′) |
| 1622 | CGCGCTTAATGCGCCGCTACGAATCAGAATCGGAACTACCGGGG |
| 1623 | AGTACATATTAACCTCCGGTTTTTTTAGGTTGGGTTATCGCGCA |
| 1624 | TCCGTGAGCATTAATTGCGTTGCGGGTCACGCCGGGTTACCTGCAG |
| 1625 | ATTAGGCAGGTCAGACGATTGGCCAAAGCCAGATAGCCCCCGTCGA |
| 1626 | TATGAGCCGCTCACTGTCACTGCGCAGCACGCGCAACAGCTGA |
| 1627 | ATCATTACCGATAACTCATCGAGTTTTTACAAGCATATAACTA |
| 1628 | TAACGGATTCAGATGATGTATCACGAACCGCCCACAAACGCCGCCA |
| 1629 | GTCACCAGAGCCGCCACCATTTTTAACCACCACCAGAGCCGCC |
| 1630 | AGAACGCGCAATCAATAATCGGCTGAACCTCCTTCATCGTAGG |
| 1631 | CGACTTAGTACCGCAATAAAGAAATTGATTCGACTTGC |
| 1632 | CCAAGTTTTTAACGGGTATTAATTCTAAGTTTTTACGCGAGGC |
| 1633 | TGCGGCTGGTAATGGGAATTCGTAGGCCAGAATGCGTTTGAGGGTTG |
| 1634 | CCTCAGAGACTGGATTATACTTCTTTTTGTTATCCCAATACAAAATA |
| 1635 | CGTTCCTGGTAATATCATACCTACCCTCGTTAAGGGCGCG |
| 1636 | ACATCGGGTACACTGGTCTTTGCTCGTCATAAAAAGAAGAAGATAAGT |
| 1637 | GCCACCACCCTCATCAATAGAAAATTTTTTTATATGGTTT |
| 1638 | GGAAGGGCGATCGGTGCGGGCCTCTTTTTTTGCTATTACGCC |
| 1639 | AATGGAAACTAATTCTGTCCAGATTTTTGACGACA |
| 1640 | GAGAGAATAACATAAAAGCTGGCGATATAATTTTTAAGAAACGCAAA |
| 1641 | TTACATTTAACAATTTCATTTGAATTTTTTTCCTTTTTT |
| 1642 | GCGCAGTGCCCGCTTTTGAGACGGGTGCCTGTCCGGGTCCAC |
| 1643 | TGTGTTCAGCAAATCTAACAGTGTGTTTTTATTGCAGTAAGCGTCAT |
| 1644 | AGTTTATTTTTTTTGTCACAAGGCGATTAAGAAC |
| 1645 | CAGAACAAACGGTACGGAGGCCACCACTCTGTCATCAGATTGTTGCCC |
| 1646 | TAGAAGAACTCAAACCTTTGATTAAATTAACCGTTGTAGGTTAACGG |
| 1647 | TCCTCATTTTGATATTACCCTCAGAACCAGCACCATATCTAACAGTA |
| 1648 | AAGCCGTTTTTCGTAGATTTTCAGGTAGAAACCCTGTTTATCAACAAT |
| 1649 | TTATTCATCAAACATCAAGAAAACAAAATTAAGCTAATGCGTTACAAA |
| 1650 | ATAAAGGTGGCTTGGGTAACGCCACCTTTACAGGGAGGTTTTGAAGCC |
| 1651 | CCCGTATAGGGTCAGTGCCTTGAGCAATACTTTATCGGCCTAATCAGT |
| 1652 | AACAGGGAAGCGCATTTTTTAGACGGGAGAATTAACTGAA |
| 1653 | GCTGGTAGGGCGAAAAACCGTCGCGCCATGAAGGAGCG |
| 1654 | CTCCCTCAGACATAGCCCCCTTATTTTTTAGCGTTTGCCA |
| 1655 | GGAATAGGATATACAGAAAATTATTATTATTTTAACGTCA |
| 1656 | TACAATTTTATCCTGAATCTTACCTGCCAGTTCCAAATAA |
| 1657 | CGATGGCCTTTTGGGGTCGAGGTGTCTTCGCGGTTTCTGC |
| 1658 | ACGAGCATGTTTAACGTCGCCTGATGCTCAGTTTCGGAAC |
| 1659 | TATCAGGGGGGGGAAAAGCACTAAGCGGGAGCAGACAGGA |
| 1660 | GCAATTCATCAATATAGCGGAATTTGCTTTG |
| 1661 | ATCGGCATCTATTATTCTGAAACAGAGGCGAAAATACCAA |
| 1662 | AACGGATAGAAACGATTGAATAATGGAACCCTACTATGGTATCATCAAGAAACCA |
| 1663 | CCGTAACACAAGCCCAATAGGAAAATCCTGATTGTTGTTGTGGAGCCGCCACGGG |
| 1664 | GGATCCCCCCTGCGGGCCGTTTTCTGAGAAGCGTGCTTT |
| 1665 | GTTTTAGCGTCTTTCCTTATCATTATAAACAACATGTTCA |
| 1666 | CCGATTTAAGATGATGGGAAACAATCGGCGAAACGTACA |
| 1667 | AAAACAGACACCCTCACGTACTCAGGAGGTTTCGGGGTTTTTGCTTTG |
| 1668 | TGATGAAATTCAATTACCTGAGCACTGCCTATACCAGGCGTACAGGAAAATAAA |
| 1669 | TTTCGGTGTGTACTGGTAATAAGTTTGATGAGATAAGTG |
| 1670 | GAAGCATAAAGTGTAATTGTTATCCGAGCTCGTAAAGGTT |
| 1671 | ACATGGCTTTTTAACGAACAGTTAATGCCCCACATCCCT |
| 1672 | TGACAGGAGGTTGTTGACGCTCAACGGAACAATATTCCTGATTTGGCAA |
| 1673 | AAAGGGGGATGTGCTGTGCCAAGCAATAGCAGGGGTTTTC |
| 1674 | GAGGGGTTAGAACCTACGTATAAATATAAGTGAATTTAC |
| 1675 | ATTAGGATTAGGAGCCATTTTTTTGGGAATT |
| 1676 | GTAAAGTTACATCCTCATATGACCAACTTTGATTTTTGCGCGTTTTA |
| 1677 | GAACCCTTTACATTGGCAGATTTTAAAAGCCTTTGCCCGAACGTCAGCGGAT |
| 1678 | GTCATTGCAGGCGCTTTCTTTTTCACTCAATCCGCCGGGCGCGGTTGCGG |
| 1679 | CTTATAAACTGATTGCAAAAAAATAACAATTCGACAAACCACCACACCCG |
| 1680 | CAACTAATCCATTGCCATTAAAAATCCTGA |
| 1681 | CCGAACACGATTGAGGACCTGCAACGTGAGGCGGTCAGTATAGTACGGT |
| 1682 | AAAAGCCGCACAGGCGGCCTTCAATAAGAGGATCGCGTCTAATAGGAA |
| 1683 | CAGCGTGGTGGTCAGTCAAATGAAGAACGAA |
| 1684 | GAAAGAGGCGGACTTGTAGAACGTAGCCTCCGAGCACTAAACATTTGA |
| 1685 | CTTTACACCCGTAAACAAACTTAAAAAGAGACGCAGAAAGCTTTCAT |
| 1686 | CTGGAGGTGTCCAGCATCAGCGGGATGAATCGAGCAACCGTCAGAC |
| 1687 | CCACCAGCAGAAGATAAGCCCTAACAACAGTT |
| 1688 | TGGTGAAGGGATAGCTTTCCAGTTATCGACATCGTTCCGGTAGATAAT |
| 1689 | CAACATTAGGCCTTCCTGTAGCCAAAAATAATAGCCCGAAACACGACC |
| 1690 | AGAAAGGAAGGCGGTTGTGTACTGGAACAAGAAATCGGCAAAATCC |
| 1691 | GAATAAAAGGGACATTCTGGCCAAAAGAATACTAAAATAGAAAGGA |
| 1692 | TGAATGGCTATTAGACCATTAGATCGCGAGCTATTCGAGCAAAAAGAT |
| 1693 | AGATTTAGTAGAGCTTAAGAGGTCAAAGAGGA |
| 1694 | CAAGCGCGTAAAGCTCAACATGTTGTTTCATTC |
| 1695 | TATTACTTTTTGCCAGAGATTAGATCAATATCTGCTGGTCAGCACCGT |
| 1696 | TGCCCTTCACCGCTTTTTTCGTCTCGTCGCTGGC |
| 1697 | GATCCAGCCGGTGCTTTTTCCCTGCACAAGAATG |
| 1698 | AATTGCTGCAGACGGTCCCCCAGCTAAACGGGTAAAATACGTAATGCC |
| 1699 | TGGATTATAACAGGAAAATTTTTCGCTCATGGAA |
| 1700 | AACATCGCTCTGACCTGAAAGCGTCAGAGATAGGATTTAGAAGTCTT |
| 1701 | TTTGTCTTTAATGCGCGAACTGATAAACAGAGAGTGCCACGCTGAGA |
| 1702 | AAGAAGTAAACAGTAATGTGAGCGAGTAACAACCCGTCATAACC |
| 1703 | TTTGAGTAACATTATTTTTCATTTTGTCGTCTGAA |
| 1704 | ACTACGAATACACTAAAACACTCTAACACCGGGATGGCT |
| 1705 | GACACGAACTAACGGGTAAGAGCAACACTATCGGATTCTCATTGAAT |
| 1706 | GTGTAGCGGTTTTTCACGCTGCGCCGCCAGCAGTTGGGGTTTCTCCG |
| 1707 | CCGTGGGAACAAAGCATAAACAACATTATTACAGGT |
| 1708 | TTGTGAGAACGTGGACTTTTTCCAACGTCAATTGCCCCAGCAG |
| 1709 | AAATCTAAAGCATCACCTTGTTTTTTGAACCCAGCGGT |
| 1710 | AAACAAAGATGTTACTCCCAAATCATTTTAACGCATAGG |
| 1711 | AGAGGCTTTTGTAGTGATGAAGGCAAATATCAGGTTATC |
| 1712 | GCCCTGAGTTTTTGAGTTGCAGCAAGCTAAGCCGGCGAACGTGGC |
| 1713 | CCAGTCGGGAAACCTGTTTTTTGTGCCAGCTGCATTA |
| 1714 | TTAAATATGCAACTAAATCTTTGACAATCATAAGGGAA |
| 1715 | CGATTAATTTTTGGGATTTTTAAACATTTTTGAGGCGCCGTCAA |
| 1716 | TAGCCGGAAGACCAGGGAACTGGCCTACGTTAATAAAGAT |
| 1717 | CATTGTGAATTACCTTATGCGAACGTAACAAAGCTGCTTTGAGG |
| 1718 | AGGGGGTAAAAAACCAGAACCAGAAATTGCTCCTTTTGAT |
| 1719 | TACAACGGACTAAAGACTTTTTCATGAGGAAATTCATTA |
| 1720 | CGAAAATCCTGCGCCAGGGTGGTTTTTTTTTCTTTTCACCAG |
| 1721 | CGCCTGTGCGAGTAAAAGAGTAAATATTTTTTAAACCCTCAA |
| 1722 | GAACGTGCCAAAAGAAGGCACCAACCTAAAACCGCAACCA |
| 1723 | GCTTACGGCGGTGGTGCCATCCCAGCCAGCAGTGGCAAAT |
| 1724 | CAAACGCGGTCCGCTGGTGGTTCCGAGTCCACTATTAAAG |
| 1725 | AGATAGACAAGAATTTTTAGCGATTTACCAGTCCCGGAA |
| 1726 | CTCCTGTCCATCACGCAGTAATAACATTTTTCACTTGACC |
| 1727 | GATTGTATCGTTTGCTAAACAACTTTCAA |
| 1728 | GGGTGAGAAAGGCCGATAGCGATTTAAACAGCTTGATAC |
| 1729 | TCCACAGACCGATTGAATACAGGCAAGGCACCACCCTCAG |
| 1730 | AGAGGGTAGCAGTATGTAACGGAAAGGTGAATCAAAATCACCGGAACCG |
| 1731 | TTTTTAGAACCCTCATATATTTTAAGCCTTTATTGCCTG |
| 1732 | AGGTCAGGAAAGGCTCTTTCACGTTGAAAATCGCGTCCAATACTCTGCC |
| 1733 | TCATAGGTGACCACCGTAATCAGTACTGTAGCGCGTTTT |
| 1734 | TGAGATTTTTGAGATGTCAGGACGTTGTGTCGAAATCCGTTTTTGACCTGCTCC |
| 1735 | AGTAAAAGGTGGCATCAATTCTTTTTACTAATAGTATTAGCTATATTTT |
| 1736 | TCTTTTCAAGCGTCAGAGCGACAGAATCAAGTGAGTCAATACTTTTTC |
| 1737 | TAAGCAAATATTTACCCTGACTATTGTACCCCAATAATTT |
| 1738 | CAAAAGGAGCCTTTAACGATAGTTCAAAGGCTCAAATCAC |
| 1739 | TCAGGTCTCGTAACCGTGCATGCGGTGTAGA |
| 1740 | TTTTAAGGGGAGCAAAATTCAAAATGCCTGAGTAATGTGTTTGCCTTT |
| 1741 | ATGTAAATGCTGATGCCTACCTTTAATCAATATATGTGAGTGAATAAC |
| 1742 | TCATTATTTTTTACCAGGTTTAATTCCCTGACGAGAAACACAGCGAAAG |
| 1743 | TAATCAAAAACGTCACACCATTACTTCTGACCAGAACTGG |
| 1744 | AGAAGGCTTATCCGGTAACCAAGTACCGCAATCCGACAAAAGGTAAAG |
| 1745 | TGAAAGTTTTTATTAAGAGGCTGACTGAGAGA |
| 1746 | CAGATATCACCCTGAACAAAGTCAACTGTTGGACACCAC |
| 1747 | AAAACATTATGACACCGGAACCAGCCCTCAGAGGAAATTAAAAGGGCG |
| 1748 | TGGGCGCACCAGCCAGCTTTCCGGTCAGGAAG |
| 1749 | CATTAGCAATATTGACGCCAAAGAATTAGCAAAATTAAGC |
| 1750 | AGGCCGGAATCCCTGTAATACTTTTGCGGGAGAAATGCAA |
| 1751 | ACGGTAATGTTTATCAGGAACAACTAAAGGAACGACAGTA |
| 1752 | CTGATAAAGCCCACGCAACCGTTCTAGCTTTTTGATAAAT |
| 1753 | CAGTTTGATACATTCAACAGCCCTCACAACGCC |
| 1754 | CAAAGACATTCATTAATACCCAAATAAATTTACGGAATC |
| 1755 | GAGACAGTATCAGGTCATTTCAACCATAAAGCTAAATCGGAATAAAGC |
| 1756 | TAACATCCTAAATTTTTTAATATTTTGTTAAATTCTGT |
| 1757 | TTGAAAACGGGAGTTAAAGGCCGCGTCGCTGAGGCTTGCA |
| 1758 | TGTTAAATCAGCTCATTTTTTTTTTAACCTCAGAAA |
| 1759 | GAAAGATAAAAGTTTTTGAATTACGAGCGGCGGA |
| 1760 | TACAAACTATAGTTAGCGTAACGATCTAAATAACCAGCGC |
| 1761 | GGCTACAGAGGCTCATTCATTTTTTGAATAAGGCTTGTCAACTTTAA |
| 1762 | AAATCCAAATAAAGTAAAGGCGTTAAATAAGACAAGCAAAT |
| 1763 | TGACCGTAATGGGATAGGCAGATACATAACGCCTCATCAGT |
| 1764 | ATTTGGGGACATTTCGCAATTTTTTGGTCAATCAATCATAGAGATCTA |
| 1765 | CAAAGAACCCATCGATAGCAGTCCTCAAGAGAAG |
| 1766 | GCAAGGATAAGATTCCCATTTTTTTCTGCGAACGAGTGTCTGGAA |
| 1767 | AGTTTGAGGGGACGATTGCGAATGGTTGATATAGCATGTAACCTGT |
| 1768 | ACCGTGTGCGCCCAATAGCAATAATTAGCAAACGTAGAAAATACATA |
| 1769 | CCTCAGAACCGCCAAGCCACCACCAGTAGCCAATGAAAGCGAGAAA |
| 1770 | AATATAATGCTGTTGCCGGATTTTTAGGGTAGCTATTTTTGA |
| 1771 | AGTGAATTCTGTAAATCGTCGCTATTAATTAATATACAAAAAATATA |
| 1772 | ATATAACAGTTAAACATCAATATGATATTCATAACCGA |
| 1773 | TTGTACCATCGATGAAGAGTCTTGAATTTCAGCTTAGATAGAATCC |
| 1774 | CCGGAATTTTTCAAACTCCAACTATAGTCATTTTTAAGCAAAGCGG |
| 1775 | AATGACAACAACCATCTCCAAAAAATTAGATTTTTAGTACCTTT |
| 1776 | ATAGTAAAATGTTTAGATTTTTTGGATATTGGGAAGATTTTTAAAT |
| 1777 | TAAGACTCTTCCAGACGCCATTCGCCATTCAGGCTGCGC |
| 1778 | AAGAGAATTCGCAAGAAAATACCGAGAGCCAGTATCACCGTCAC |
| 1779 | AGCCCCAAAAACAGGAATAGAAAGCTTGCTTTCGGTTAATTTCATC |
| 1780 | TTGTATCGCGTAAAACATCAGAAACTCAGAGGTAGCAT |
| 1781 | AGATTTTTTTGTATCATCGCACAGCATCGGAACGAGGGTAGCAA |
| 1782 | AGAAACAACCTGAACAAGAAATACCGCTCACAATTCCACACAAAACT |
| 1783 | TTTCCATCCTAATTTTTAAATCATTGCACGTCCAGTCAC |
| 1784 | GGGAATAATATCCCATACGAGCCGAACAGCCAAGATTAGTTGCTAAGTT |
| 1785 | TAATTTTTGCACCCAGCAAAATGAATTTCAGAGGTAAAACG |
| 1786 | TTTTACTCACATAGCTGTTTCCTGTGTGAAAAGCCTGGGCAGTTGACACCCAA |
| 1787 | GGTGCTGCATCATGGTCCTCCTCAGTGCCTAATGAGTGAGCTATCAT |
| 1788 | TTTGTTTCCAGAGCCTAATTAACGCTAATGAACCATACCTCACC |
| 1789 | CCAGAATCCACGGTCACCTAAAGGATCAAGTTCACTACGCGAGCGTCTTCT |
| 1790 | TTCTTGACAAGAACCGGATGTTTCCATGATTATAC |
| 1791 | ATTGCATCTTCAAAGCAAATAGCGCAGATGAACCTTCATCAAT |
| 1792 | TTACCAGACCTGGCTGACGGTGTACACGAGGCG |
| 1793 | ACGAGAATGAATGCTTTTTTGCCAGCTCACGT |
| 1794 | CCAACGGCTGGTCAGCGCCAACGCGCT |
| 1795 | TAGGCGGTTTGCGTATTGGGTTTGATG |
| 1796 | TCTTTAGGGCCAGAGCAAACGATGTCAAAAGAATT |
| 1797 | TAGATAGGGTTGAGTGTTGCTCACGGAAATTTCTG |
| 1798 | TTTTTATCAGAGAGATAAAGGAAACGATAAACACATGGTTTG |
| 1799 | TTTTTGTAAATTGGGCAGGAATACCACTTTTT |
| 1800 | ACCAGAAGCCGAAGCAATGAAATAGCAATAGATTGAGCGCTAATTTTTT |
| 1801 | TTTCGAGAACATGTAATTTAGGCTTCTTACCAGTATTTTTT |
| 1802 | ATGGGATGCCCACCGCTTCTGGTGCCGGAAACCAAGAAAAGTAAGTTTTT |
| 1803 | AGAGGCATATAATTACAACAGTAGAATTGAGTTAAGCCCAATAATTTTTT |
| 1804 | TTTTTATTCAACTAATGTCACGTTGGAATCGTCATTTTTTAATATT |
| 1805 | TTTTTATTTAACAACGCCCCAGTAATCTTGCTTTATCAAAA |
| 1806 | TTTTTAAAGCCAACGCTCTAGAAAAAGCCTGTTTAGTTTTTT |
| 1807 | TTTTTAAGAGCAAGAAACCCTTTTTAGGCAAAGCGTTAGTAAATGAATT |
| 1808 | CTATCTTAGAAACCGCCCACAAGGGCTTAATTGAGAATCGCCATTTTTT |
| 1809 | TTTTTCAGATAGCCGAACAAAGTTCATCAGCGGAGTGAGGAAGGTAACAAGAGAA |
| 1810 | TTTTTATCATATGCGTTTTTCCCTTTAAGACGCTGAGAAAGGTAAAG |
| 1811 | GCGCCGACTATATTCGTTTTGCGGGATCGTCACCCTCAGCCAGAACGAGTATTTTT |
| | H1 design oligo sequences (5′-3′) |
| 1812 | GCAGTAGAGTAGGTAGAGATTAGGCACCAGAAGGCCCATGTAGCCAGCATACGGCCAC |
| 1813 | GCAGTAGAGTAGGTAGAGATTAGGCAACGGCCAGCAATTTTCAGGGATAGCTGAGTTT |
| 1814 | GCAGTAGAGTAGGTAGAGATTAGGCAGGCGCTAGGGCGCATCGAGCTTGA |
| 1815 | GCAGTAGAGTAGGTAGAGATTAGGCACGCCATCATATTAATTCACCAGTCAGAATTAGA |
| 1816 | GCAGTAGAGTAGGTAGAGATTAGGCACTCATTTGCGTACTCGTATTAAAT |
| 1817 | GCAGTAGAGTAGGTAGAGATTAGGCACCCCCTCAACCATAATTTTTTCAAAAA |
| 1818 | GCAGTAGAGTAGGTAGAGATTAGGCATGTAGCATATTCGCATAATAAATCGGGAGGAA |
| 1819 | GCAGTAGAGTAGGTAGAGATTAGGCAATCGCACTTTTAAATTGTAAACG |
| 1820 | GCAGTAGAGTAGGTAGAGATTAGGCAGGAAGTTTTGTCGTCTCTTATTAC |
| | DNA sequence complementary to DNA handle of |
| | H1 to H3 design oligos |
| 1821 | TGCCTAATCTCTACCTACTCTACTGC |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

The following Examples illustrates the invention described above, but is not, however, intended to limit the scope of the invention in any way. Other test models known as such to the person skilled in the pertinent art can also determine the beneficial effects of the claimed invention.

### EXAMPLES

### Introduction

Effective broadband antiviral platforms that can act on existing viruses and viruses yet to emerge are not available, creating a need to explore treatment strategies beyond the trodden paths. Here, we report virus-encapsulating DNA origami shells that achieve broadband virus trapping properties by exploiting a widespread background affinity of viruses to heparan sulfate proteoglycans (HSPG). With a calibrated density of heparan sulfate (HS) derivatives crafted to the interior of DNA origami shells, we could successfully encapsulate adeno-, adeno-associated-, chikungunya-, dengue-, human papilloma-, noro-, polio-, rubella-, and SARS-CoV-2 viruses or virus-like particles, in one and the same HS-functionalized shell system without the need for virus-type-specific binders. Our HS-functionalized shells amplify the individually weak and reversible interactions of HSPG to viral surfaces through strong avidity effects that emerge when curvature-matching HS-coated shells engulf the virus particles. Depending on the relative dimensions of shell to virus particles, multiple virus particles may also be trapped per shell, and multiple shells can also coordinate and enclose clusters containing dozens of virus particles. Since steric occlusion in virus-engulfing shells can prevent viruses from interactions with host cells, the heparan sulfate-coated virus-engulfing shells open an attractive path for establishing a broadband antiviral treatment strategy.

### Example 1: Shell Design and Synthesis Principles

Here, we address the challenge of creating a broad-spectrum antiviral by exploiting the conserved background binding of HSPG to viruses to irreversibly trap viruses in HS-functionalized neutralizing shells (FIG. 1A, right panel).

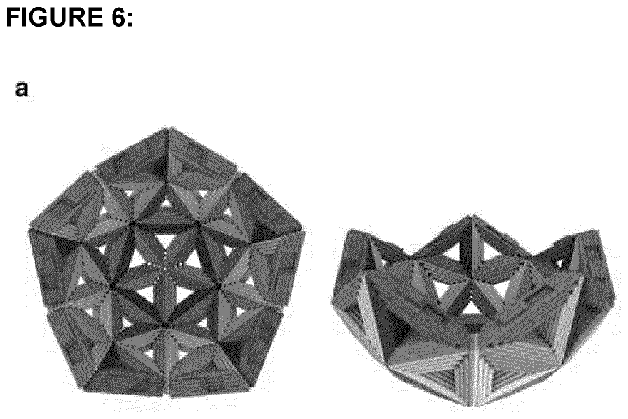

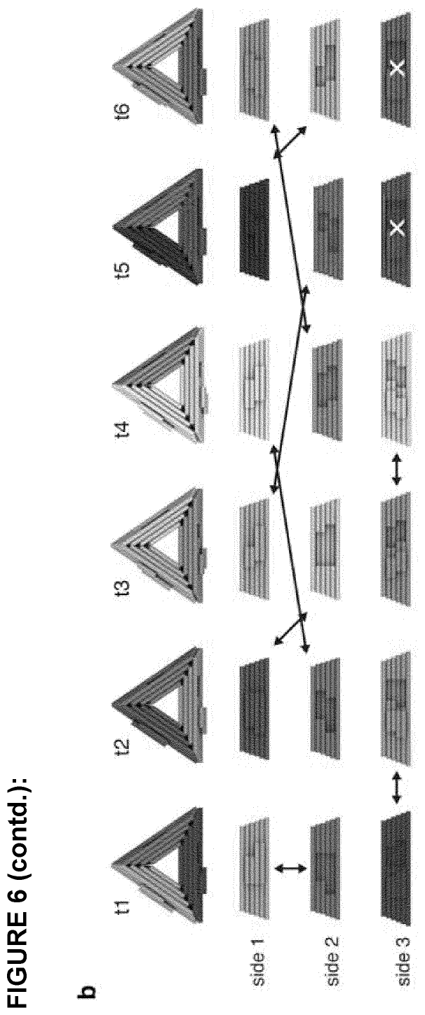

To capture differently sized viruses, we fabricated three DNA origami shell variants and functionalized their interior with the same HS derivative. We used the previously described octahedral and T=1 icosahedral half shell designs (O and T1, respectively; Sigl et al., loc. cit.) featuring 40 nm and 85 nm wide cavities, respectively (FIG. 1D). We also developed a new T=3 icosahedral half shell design, termed T3, for the encapsulation of larger virus particles that do not fit into O and T1 shells (FIG. 1E). The T3 design is a finite-size higher-order assembly consisting of a total of 30 triangular subunits, partitioned as five copies of six different full-size DNA origami triangle designs with specific edge docking rules (FIG. 6). The resulting shell has a cavity diameter of approximately 150 nm. Negative stain transmission electron microscopy (TEM) images validate the successful assembly of T3 shells (FIG. 1F and FIG. 7).

Staple strands for origami folding reactions were purchased from Integrated DNA Technologies (IDT) and used with standard desalting purification unless stated otherwise. DBCO-modified handle strands were purchased from Biomers at HPLC grade. Azide-modified heparan sulfate derivatives were purchased from Glycan Therapeutics (catalog references: 1a: GT24-AZ-021; 1b: GT24-AZ-005; 1c: GT18-AZ-003; 1d: customized product). VLPs were purchased from The Native Antigen Company, Creative Biostructure and Creative Biolabs (catalog references can be found in Table 12).

TABLE 12
VLP providers and catalog references.

| VLP | Provider | Reference |
|---|---|---|
| Poliovirus type 3 | Creative Biolabs | VLP-003YF |
| Dengue type 1 | The Native Antigen Company | DENV1-VLP-100 |
| Norovirus G II.4 | The Native Antigen Company | REC31620-100 |
| HPV 16 | Creative Biostructure | CBS-V641 HPV16 |
| Chikungunya | The Native Antigen Company | CHIKV-VLP-10 |
| SARS-CoV-2 | Creative Biolabs | VLP-050YF |
| Rubella | The Native Antigen Company | REC31651-100 |

Folding of DNA origami triangular subunits

DNA origami structures were folded in one-pot reactions containing 50 nM of single-stranded scaffold DNA (M13, 8064 bases) and 250 nM of each staple strand in a standardized “folding buffer” (FoBx) containing x=20 mM MgCl₂, 5 mM Tris Base, 1 mM EDTA and 5 mM NaCl at pH 8.00. Scaffold M13 was produced as previously described based on M13 8064 as scaffold sequence (SEQ ID NO: 1; Engelhardt, F. A. S. et al., ACS Nano 13 (2019) 5015-5027). All folding reactions were subjected to optimized thermal annealing ramps (Table 13) in a Tetrad (Bio-Rad) thermal cycling device. It should be noted at this point that any variant of M13 8064 or in fact any other single-stranded DNA of sufficient length could have been used as scaffold sequence together with a correspondingly designed set of staple strands. Alternatively, DNA origami structures of the type used in the present application could be constructed by using different sets of overlapping single-stranded oligonucleotides and standard DNA origami techniques.

TABLE 13
Temperature ramps and scaffold used for each DNA origami
triangle subunit. For scaffold sequence see SEQ ID NO:
1 in Table 1. For staple sequences see Tables 2-10.

| | Denaturation | Temperature | Storage | |
|---|---|---|---|---|
| | step (15 min) | ramp (1° C./1 | temperature | |
| Structure | (° C.) | h) (° C.) | (° C.) | Scaffold |
| T_octa | 65 | 60-44 | 20 | M13 |
| | | | | 8064 |
| T1 (pentamer | 65 | 58-54 | 20 | M13 |
| triangle) | | | | 8064 |
| T1 (ring triangle) | 65 | 65-52 | 20 | M13 |
| | | | | 8064 |
| T3 (6 triangles) | 65 | 56-52 | 20 | M13 |
| | | | | 8064 |

### Purification of Triangle Subunits and Shells Self-Assembly

All origami structures were purified using agarose gel extraction (1.5% agarose containing 0.5×TBE and 5.5 mM MgCl₂) and centrifuged for 30 min at maximum speed for residual agarose pelleting. If the origami needed a concentration step, ultrafiltration (Amicon Ultra 500 μl with 100 kDa molecular weight cutoff) was performed prior to shell assembly. For shell assembly, the purified triangles were mixed in 1:1 ratio. Typical triangle subunit concentrations ranged from 5 to 400 nM, while assembly times depended on the shell type. Table 14 summarizes and offers a comparison on the optimized salt concentrations, temperature, and self-assembly times required for all shells used in this study.

TABLE 14
Half-shells assembling conditions.

| Structure | [MgCl₂] (mM) | Temperature (° C.) | Time | |
|---|---|---|---|---|
| O shell | 40 | 40 | overnight | |
| T1 shell | 40 | 40 | 24 | h |
| T3 shell | 25 | 40 | 8 | weeks |

The assembled shells were UV cross-linked for 1 h at 310 nm using Asahi Spectra Xenon Light source 300 W MAX-303 (Gerling, T. et al., Sci. Adv. 4 (2018) eaau1157). Buffer exchange to 1×PBS containing 10 mM MgCl₂ was performed prior to VLP encapsulation experiments using ultrafiltration (Amicon Ultra 500 μl with 100 kDa molecular weight cutoff) or dialysis (D-Tube™ Dialyzer Mini, MWCO 12-14 kDa, 2×500 ml exchanges over 8 h, r.t).

### Heparan Sulfate Attachment to DNA

We used a strain-promoted azide-alkyne 1,3-dipolar cycloaddition reaction (SPAAC) to covalently attach a heparan sulfate derivative to a DNA oligonucleotide (FIG. 1B, FIG. 5) which can hybridize to specific acceptor sites in the interior of the DNA origami shells: single-stranded DNA extensions termed “handles”. For the coupling, we used azide-group modified HS derivatives containing either 8 or 18 saccharide monomers (1a and 1c, respectively), including monomers such as N-acetyl-glucosamine and glucuronic acid which characterize HS polymers. As controls, we used 8-mer and 18-mer polysaccharides lacking the sulfate and sulfonate groups (1b and 1d, respectively). The DNA oligonucleotide to be clicked to the different HS polymers was modified with a dibenzocyclooctyne (DBCO) moiety (2), and the SPAAC reaction occurred rapidly upon mixture of both components. We analyzed the reaction products (3a-d) by polyacrylamide gel electrophoresis (PAGE), which revealed different electrophoretic mobilities for the different product versions consistent with expectation (FIG. 1C). Higher molecular weight reaction products had slower mobility, and the sulfate-containing products migrated faster in the gel compared to the products lacking the sulfate groups, which we attribute to the additional negative charges.

Excess of azide-modified heparan sulfate derivatives (1a-d) were mixed in a 4:1 ratio with DBCO-modified DNA to form the respective products (3a-d). MgCl₂ was added to a 0.5 M concentration and the mixture was left overnight at 37° C. to achieve >90% conversion. The products were run in a preparative 10% PAGE gel for 2 h at 35 W. Subsequently, the product bands were cut away and were crushed. 1×TEN buffer (10 mM Tris-HCl, 1 mM EDTA, 100 mM NaCl, pH 8.00) was added to dissolve and recover the modified oligonucleotides, and EtOH precipitation was used for concentration and buffer exchange. The pure products were redissolved and kept in double distilled H₂O at either 4° C. or −20° C.

### Attachment of Heparan Sulfate-Modified DNA Constructs to DNA Origami Shells

We then hybridized the HS-modified DNA oligonucleotides to sequence-complementary single-stranded DNA handles protruding from the target DNA origami shell's interior surface.

### Testing of Different Heparan Sulfate-Coupled DNA Origami Shells

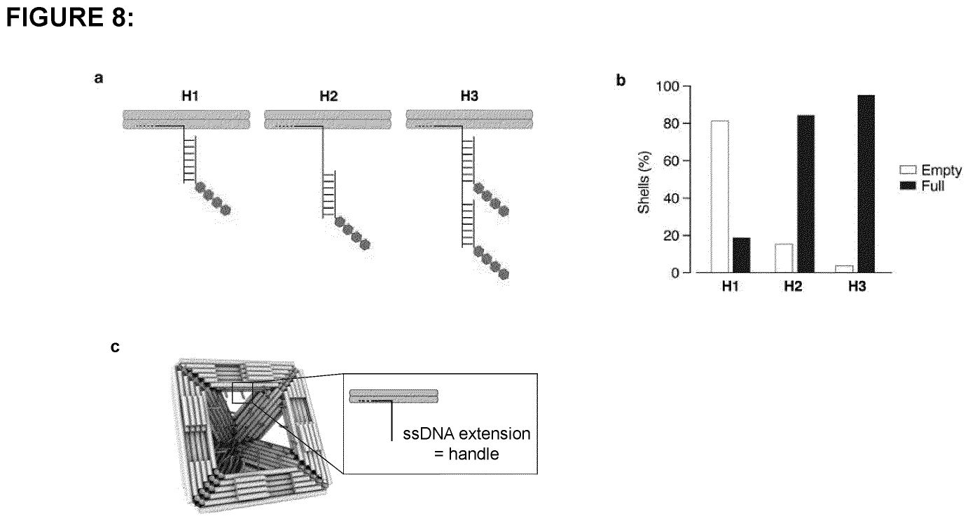

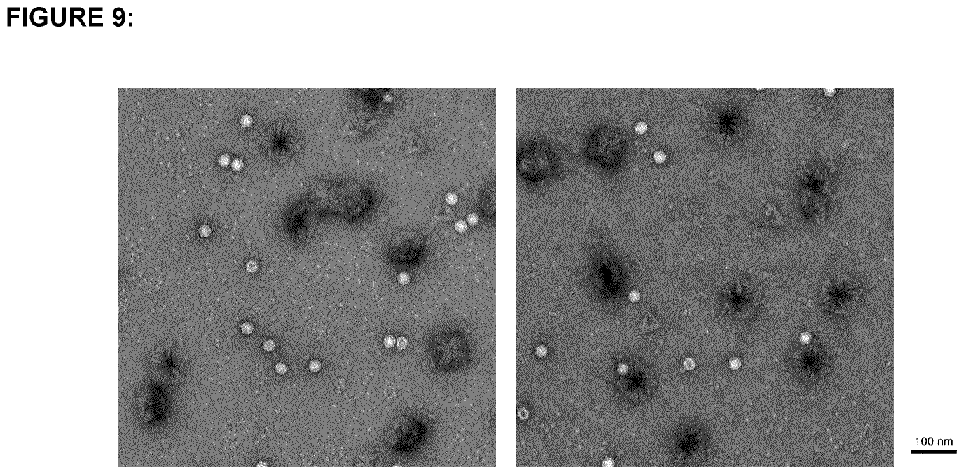

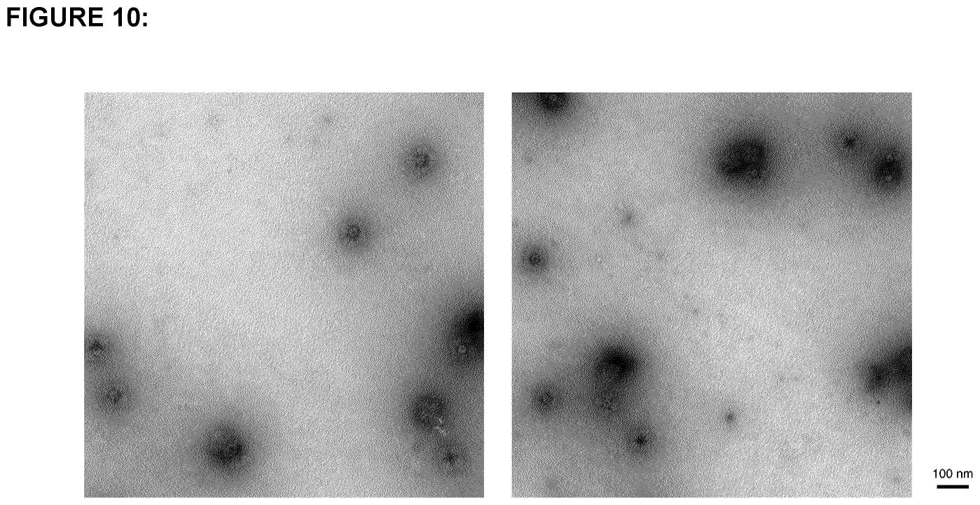

In initial experiments with adeno-associated virus serotype 2 (AAV2) we explored three different DNA handle designs: proximal (H1), distal (H2), and branched (H3) to determine the type and density of the HS modifications required for efficiently trapping viruses (FIG. 1G). These initial experiments with O half-shells showed that H1 was least efficient, and both H1 and H2 designs were not as efficient for virus trapping as the H3 branched handle design. Samples were analyzed via negative stain transmission electron microscopy (TEM), where images were collected using an automated montage setup to minimize bias. Blind TEM quantification of particles revealed about 96% of shells to be occupied with AAV2 when H3 was hybridized to the 18-mer HS derivative (3c), improving from the about 30 and 84% of occupied shells achieved with H1 and H2, respectively (FIG. 8). We therefore used the branched handle design H3, and the HS 18-mer variant (3c) henceforth, unless otherwise specified. We confirmed that the interaction with AAV2 is due to the sulfate and sulfonate groups present in the HS structure, as the 3d HS derivative used as negative control did not demonstrate any binding (FIG. 9). Importantly, all AAV2 particles were trapped with O shell excess (FIG. 10).

### Example 2: Trapping of Different Viruses by DNA Origami Shells

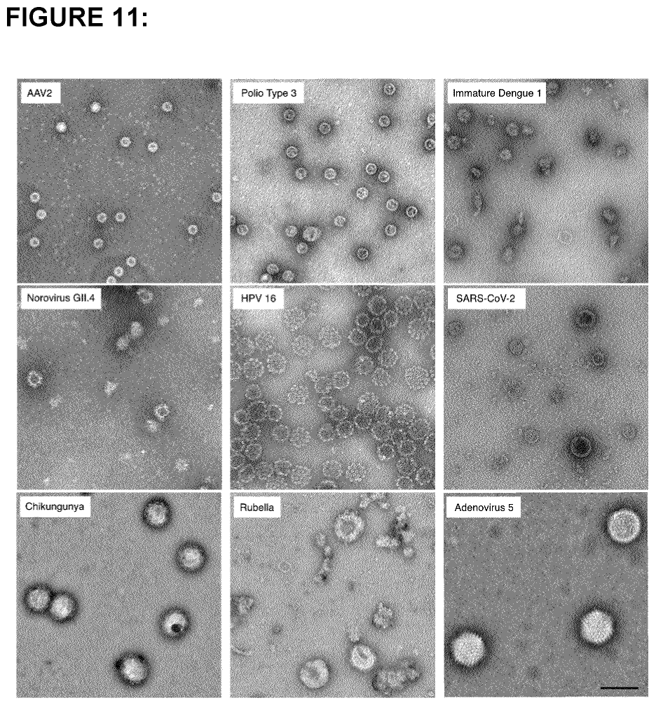

With the HS handle design thus established, we tested the HS-modified DNA origami shells for their ability to trap a variety of exemplary viruses and virus-like particles (VLPs) (Zeltins, A., Mol. Biotechnol. 53 (2013) 92-107). Our target virus library sampled enveloped and non-enveloped particles, particles from different viral families, and particles with dimensions ranging from 25 to 90 nm (Table 11, see also FIG. 11 for TEM images).

### Maturation of Dengue VLPs

Dengue VLP maturation was adapted by published methods (Yu, I.-M. et al., Science 319 (2008) 1834-1837; Yu, I.-M. et al.; J. Virol. 83 (2009) 12101-12107). Briefly, dengue VLP sample (10 μl, 0.39 mg/ml, The Native Antigen Company, cat. no. DENV1-VLP) was added to MES buffer (10 μl, 50 mM, pH 6.00) and gently mixed. Next, CaCl_{2 (aq)} (0.75 μl 0.1 M) and furin (3.9 μl, 2000 U/ml, New England Biolabs, cat. no. P8077) were added and mixed, and the sample was incubated at 30° C. for 16 h. After incubation, Tris buffer (25 μl 100 mM Tris-HCl, 120 mM NaCl, pH 8.00) was added to the sample, and the sample was immediately dialyzed against 1×PBS (D-Tube™ Dialyzer Mini, MWCO 12-14 kDa, 2×50 ml exchanges over 24 h, 4° C.). Matured dengue VLP sample was used immediately and stored at 4° C.

### Viruses and VLPs Encapsulation

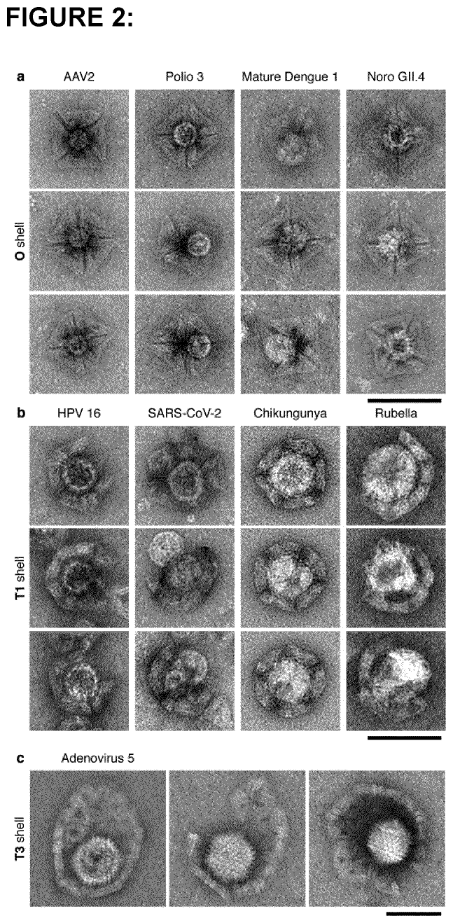

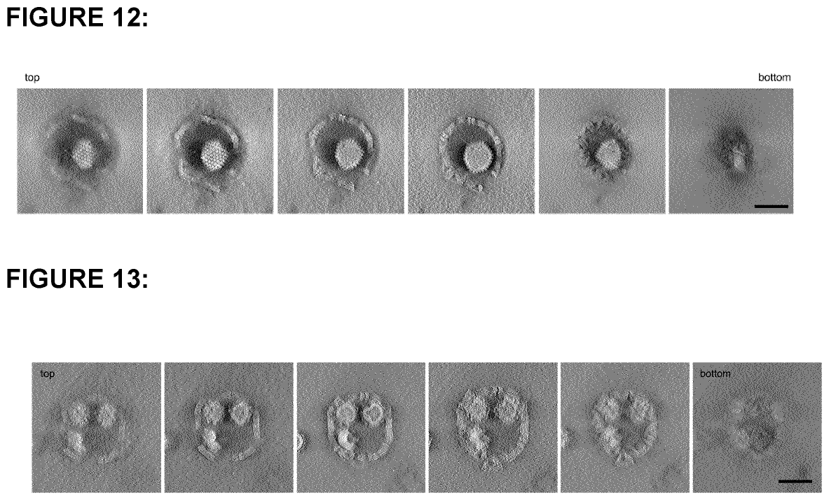

We used HS-modified O shells to sequester AAV2, poliovirus, mature dengue, and norovirus (FIG. 2a); HS-modified T1 shells to trap human papilloma virus 16 (HPV 16), SARS-CoV-2, chikungunya and rubella particles (FIG. 2b); and the HS-modified T3 shell for enclosing adenovirus 5 (FIG. 2c and FIG. 12 for TEM tomography).

Pre-assembled and UV-welded shells in 1×PBS containing 10 mM MgCl₂ were mixed with a VLP sample in the appropriate ratio to achieve either shell or VLP excess. The MgCl₂ concentration was adjusted to 10 mM and the samples were incubated at RT for 2 h. Usual amounts of sample for TEM analysis range from 5-10 μl total solution at about 10 nM triangle origami concentration. Negative stain TEM grids were prepared immediately after the 2 h incubation.

### Negative Staining TEM

Samples were incubated on glow discharged (45 s, 35 mA) formvar carbon-coated Cu400 TEM grids (Electron Microscopy Sciences) for 90 to 120 s depending on origami and MgCl₂ concentrations. Next, the grids were stained for 30 s with 2% aqueous uranyl formate containing 25 mM NaOH. Imaging was performed with magnifications in between 10,000× and 42,000× in a SerialEM at a FEI Tecnai T12 microscope operated at 120 kV with a Tietz TEMCAM-F416 camera. TEM micrographs were high-pass filtered to remove long-range staining gradients and the contrast was auto-leveled using Adobe Photoshop CS5. To obtain TEM statistics in an unbiased fashion, automatic grid montages were acquired. For detailed information on selected particles, negative stain EM tomography was used as a visualization technique. The tilt series were performed from −50° to +50° and micrographs were acquired in 2° increments.

Tilt series were processed with Etomo (IMOD) to acquire tomograms (Kremer, J. et al., J. Struct. Biol. 116 (1996) 71-76). The micrographs were aligned to each other by calculating a cross correlation of the consecutive tilt series images. The tomogram is then generated using a filtered back-projection. The Gaussian-Filter used a cutoff between 0.25 and 0.5, and a fall-off of 0.035.

TABLE 11
Target viruses and virus-like particles tested in this study.

| | | | | Genome | Approx. | |
|---|---|---|---|---|---|---|
| Virus/VLP | Family | Enveloped | Surface details | type | size (nm) | References |
| AAV2* | Parvoviridae | No | 3 capsid proteins | ssDNA | 25 | 1 |
| Poliovirus type 3 | Picornaviridae | No | 4 capsid proteins | ssRNA^{‡} | 30 | 2 |
| Dengue type 1 | Flaviviridae | Yes | 1 envelope protein | ssRNA^{‡} | 30-40 | 3 |
| Norovirus GII.4 | Calciviridae | No | 1 capsid protein | ssRNA^{‡} | 30-45 | 4 |
| HPV 16 | Papillomaviridae | No | 2 capsid proteins | dsDNA^{‡} | 35-50 | 5 |
| SARS-CoV-2 | Coronavirus | Yes | 1 envelope, 1 spike protein | ssRNA^{‡} | 30-70 | 6 |
| Chikungunya | Togaviridae | Yes | 2 envelope proteins | ssRNA^{‡} | 65-70 | 7 |
| Rubella | Matonaviridae | Yes | 2 envelope proteins | ssRNA^{‡} | 65-80 | 8 |
| Adenovirus 5* | Adenoviridae | No | 3 capsid proteins | dsDNA | 90 | 9 |

*Infectious virus;
^{‡}Data referring to the infectious virus which is modelled by a VLP in this study.
1. Liu, A. P. et al., J. Pharm. Biomed. Anal. 189 (2020) 113481;
2. Hogle, J. M., Annu. Rev. Microbiol. 56 (2002) 677-702;
3. Kuhn, R. J. et al., Cell 108 (2002) 717-725;
4. Chan, M. C. W. et al., P. K. S. 51-63 (2017) doi: 10.1016/B978-0-12-804177-2.00004-X;
5. Goetschius, D. J. et al., Sci. Rep. 11 (2021) 3498;
6. Yao, H. et al., Cell 183 (2020), 730-738.e13;
7. Yap, M. L. et al., Proc. Natl. Acad. Sci. 114 (2017) 13703-13707;
8. Mangala Prasad, V. et al., PLOS Pathog. 13 (2017) e1006377;
9. Russell, W. C., J. Gen. Virol. 90 (2009) 1-20

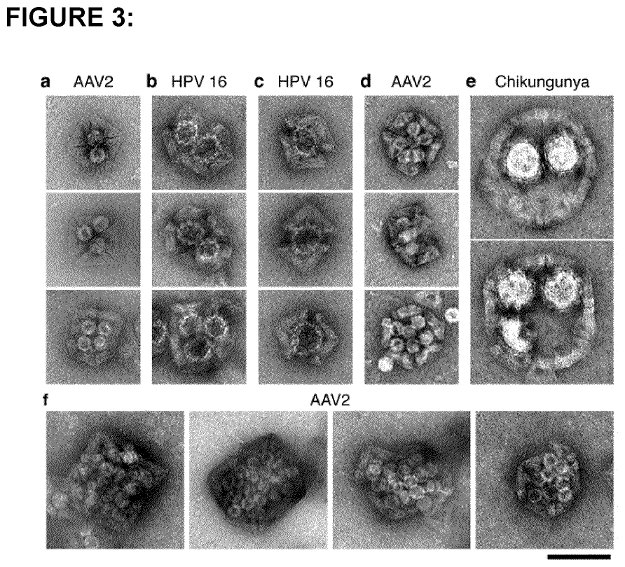

Interestingly, in many instances the multivalent interactions between the HS coating on the shell interior and the virus particles appeared sufficiently strong to support substantial elastic deformations of the surrounding shell. For example, the T3 shell material deformed from spherical to elliptical around adenovirus particles, presumably driven by maximization of the number of molecular interactions between the HS moieties on the shell interior surface and the viral surface, at the expense of elastically deforming the shell. The O shells deformed occasionally so that up to four AAV2 particles were accommodated in its cavity (FIG. 3a), even though by design the O shell has only room for one AAV2 particle if it were completely rigid. The T1 shell also flexed to fit up to three HPV 16 copies (FIG. 3b). Depending on the relative stoichiometry between shells and virus particles, we also observed sandwich-like structures where two shells coordinated one virus particle (e.g., with HPV 16 and O shells, FIG. 3c). If the shell diameter exceeded substantially the target virus dimensions, multiple target particles could be sequestered. For example, we observed up to six AAV2 per T1 shell (FIG. 3d), and up to three chikungunya in T3 shells (FIG. 3e and FIG. 13 for TEM tomography). Furthermore, multiple copies of HS-modified shells could also cooperatively encapsulate dozens of AAV2 particles in clusters surrounded and protected by DNA origami shell material (FIG. 3f). These results support the notion that the shells are flexible to adapt and capture also more pleomorphic virus particles.

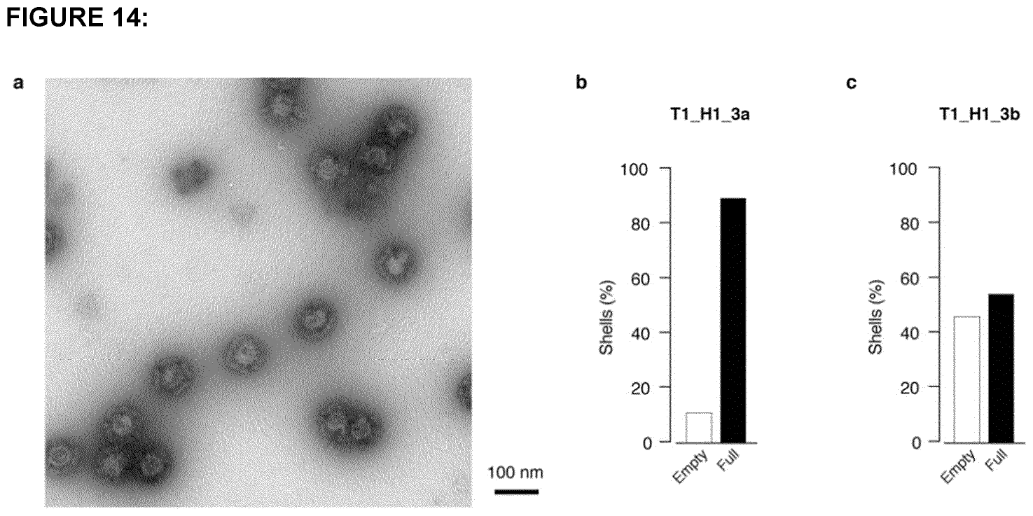

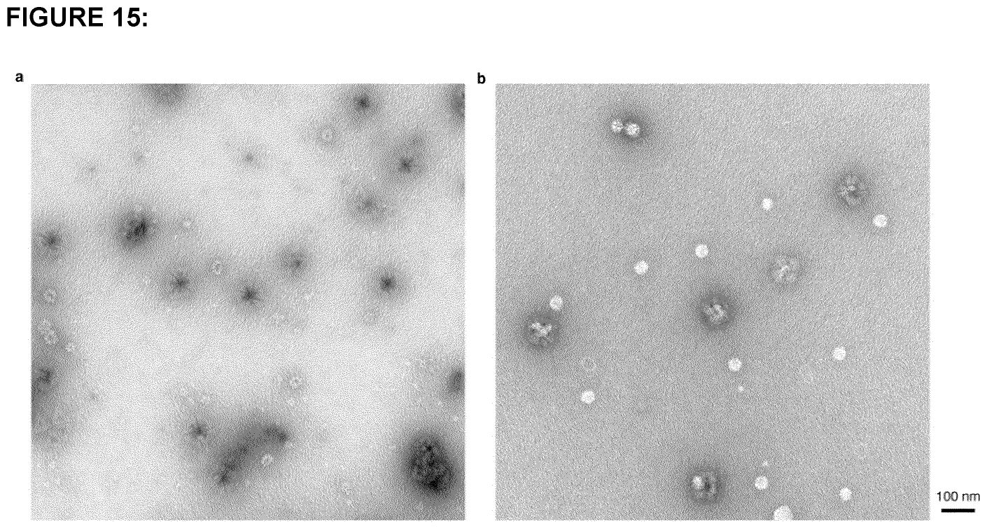

In the negative staining TEM images, we saw that the chikungunya VLP particles appeared to completely fill the T1 cavity. Presumably due to the resulting high degree of shape-complementarity, we could efficiently trap chikungunya particles within T1 shells using any of the different handle designs described in FIG. 1G in high yields (H1, 3a, 90% full shells). In fact, we could trap chikungunya even with the 3b negative control, which are shells with a coating lacking the sulfate and sulfonate groups, albeit at lower yield (H1, 3b, 54% full shells, see also FIG. 14). We interpret this phenomenon as a manifestation of molecular recognition on the mesoscale. The effect is presumably due to cooperative amplification of weak electrostatic interactions between the negatively charged DNA shells and the chikungunya particles as they interact over extended surface areas. Incidentally, this observation also suggests another route for modification-free virus trapping which considers precisely tailoring shells to the dimensions of the target virus.

Dengue virus, as well as some other viruses, present two distinct “mature” and “immature” conformations. The viral surface proteins must undergo certain conformational changes to become infectious, allowing them to move between vector and host, and/or infected and healthy cells (Yu, I.-M. et al., Science 319 (2008) 1834-1837; Yu, I.-M. et al., J. Virol. 83 (2009) 12101-12107; Lim, X.-X. et al., Nat. Commun. 8 (2017) 14339; San Martín, C., Virus Maturation. In: Physical Virology: Virus Structure and Mechanics (ed. Greber, U. F.) 129-158 (Springer International Publishing, 2019), doi: 10.1007/978-3-030-14741-9_7). While the usage of VLPs is highly convenient for safety reasons, we do acknowledge some limitations. For instance, initially our dengue VLP samples contained a high percentage of immature particles which did not bind to our HS-functionalized shells. To overcome this, we induced enzymatic maturation of the dengue VLPs, as would occur in vivo, and observed binding of the matured particles (FIG. 2a dengue and FIG. 15).

We also performed cryogenic electron microscopy (cryo-EM) measurements of HPV 16 and chikungunya VLPs trapped inside O and T1 shells, respectively (FIG. 4).

### Cryo-EM

DNA origami shells were prepared and functionalized, and viruses trapped as described above. Samples (O+HPV: 70 nM triangles; T1+chikungunya: 200 nM triangles) were incubated 60 s on glow-discharged lacey carbon 400-mesh copper grids with an ultrathin carbon film. Subsequently, the grids were plunge frozen in liquid ethane with a FEI Vitrobot Mark V (blot time: 2.5 s, blot force: −1, drain time: 0 s, 22° C., 100% humidity, 3 μl sample). Cryo-EM imaging was performed with a spherical-aberration (Cs)-corrected Titan Krios G2 electron microscope (Thermo Fisher) operated with 300 kV and equipped with a Falcon Ill 4k direct electron detector (Thermo Fisher). Automated image acquisition was performed in EPU 2.6 (dose: 42-45 e⁻/Å², exposure time: 3-5 s, 12 fractions, pixel size: 0.23 nm (O+HPV) and 0.29 nm (T1+chikungunya), defocus: −1.5 to −2 μm). Micrographs were processed in RELION-3 (Zivanov, J. et al., eLife 7 (2018) e42166) using MotionCor2 (Zheng, S. Q. et al., Nat. Methods 14 (2017) 331-332) and CTFFIND4.1 (Rohou, A. & Grigorieff, N., J. Struct. Biol. 192 (2015) 216-221). Particles were automatically picked with cryYOLO 1.7.6 (Wagner, T. et al., Commun. Biol. 2 (2019) 1-13). Extracted particle images were classified and selected by visual inspection through multiple rounds of 2D and 3D classifications. Initial models were generated in silico in RELION-3. 3D reconstructions and multibody refinement yielded electron density maps with resolutions of 26 Å for O shells trapping HPV (EMD-13884, 1× O+HPV: 7834 particles, 2× O+HPV: 4634 particles) and 36 Å for T1 shells trapping chikungunya (EMD-13883, 1259 particles, C₅ symmetry).

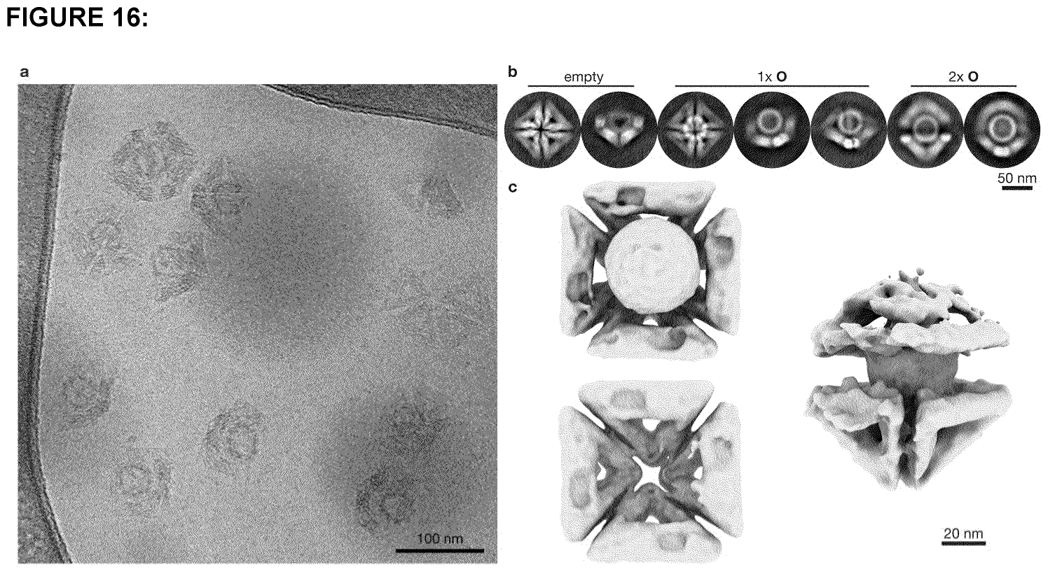

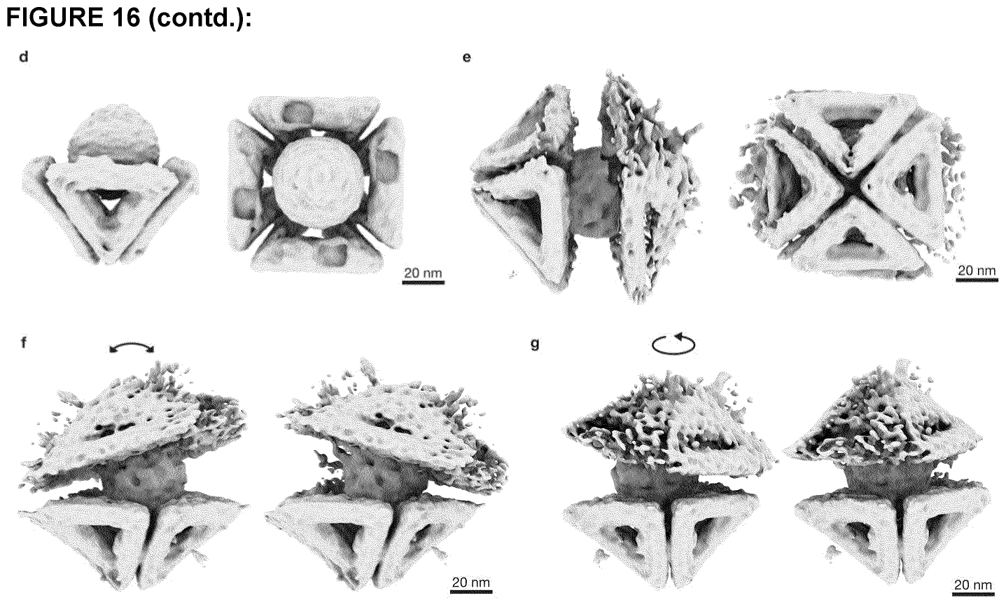

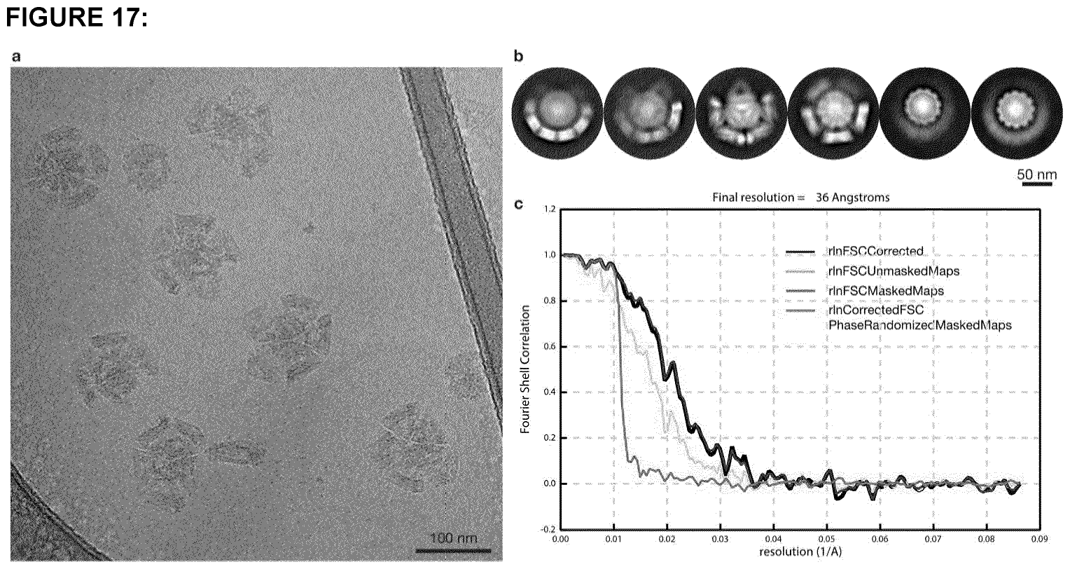

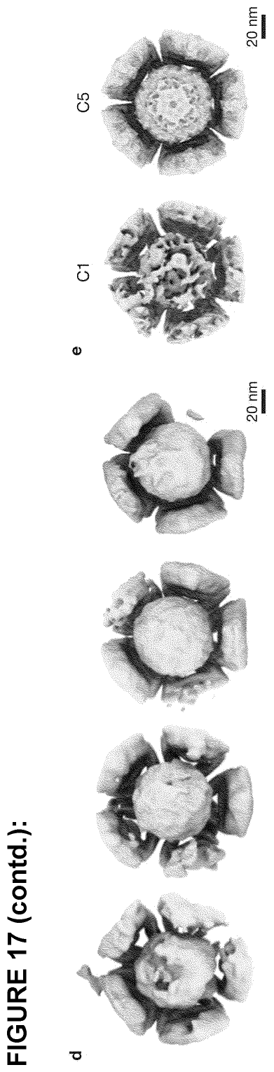

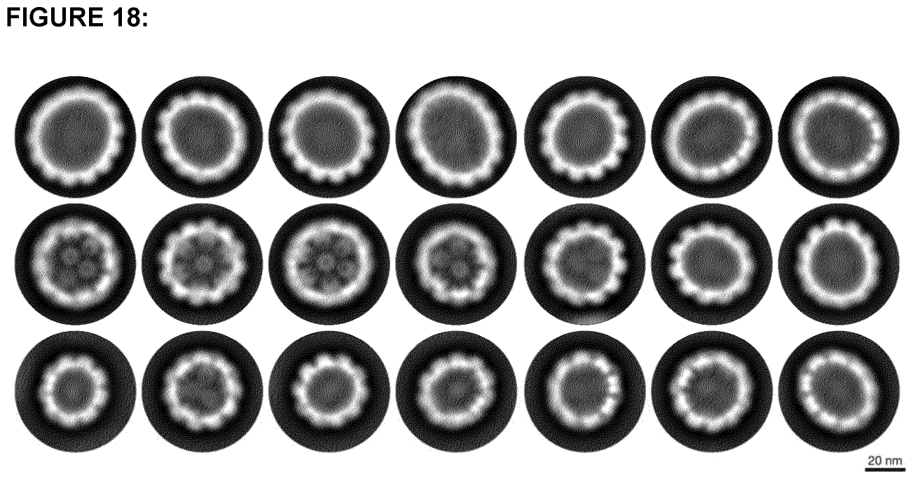

Two-dimensional (2D) class average images and 3D cryo-EM reconstructions confirmed that the VLPs were successfully trapped within the respective shell's cavities (FIG. 4b,e and FIG. 16-17). While one O shell is not sufficiently large to encapsulate an entire HPV 16 particle, two O copies can coordinate and completely cover an entire VLP (FIG. 4c). 2D class averages of free HPV 16 showed a variation in particle sizes within the VLP sample (FIG. 18). Consistently, we also found that the gap distances in between O shells (indicated by the white arrows in FIG. 4b) varied depending on whether a smaller or larger HPV 16 particle was trapped. The cryo-EM map that we determined for the complex consisting of a chikungunya VLP in a HS-modified T1 shell reveals the near-perfect fit between the two particles (FIG. 4e,f). The cryo-EM maps provide compelling illustrations of the extent of relative dimensions of the artificial DNA origami shells relative to their viral targets and the extent of surface occlusion that can be achieved by sequestering viruses in shells.

### Example 3: Stability of DNA Origami Shells Encapsulating Viruses/VLPs

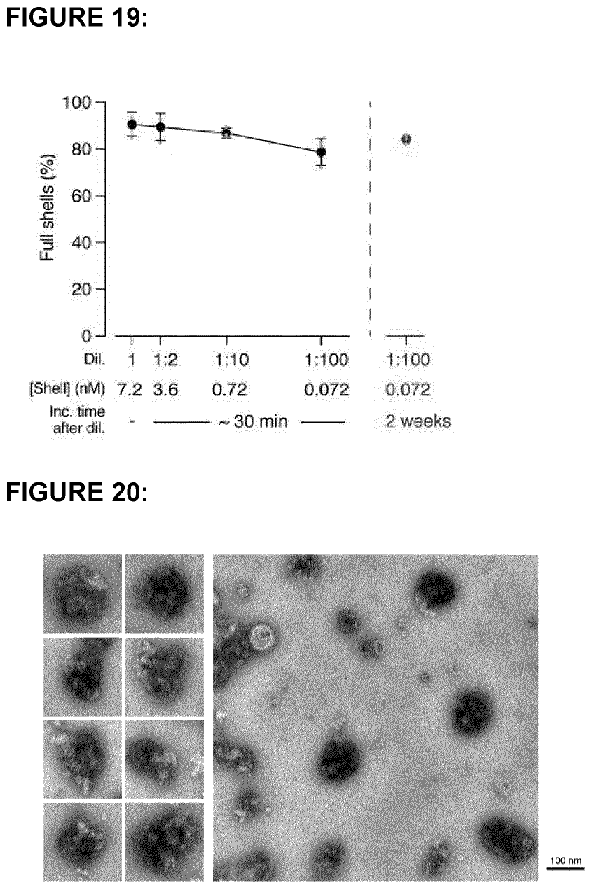

Finally, to test the stability of virus trapping by the shells, we subjected exemplarily a sample consisting of AAV2 encapsulated in O shells to a dilution series. The fraction of occupied shells remained the same prior and after 100-fold dilution and incubation for 14 days in the diluted sample relative to the non-diluted sample (FIG. 19), suggesting that the spontaneous dissociation rate for the complexes formed between AAV2 particles and the surrounding HS-modified shell is at least on the scale of weeks under the conditions tested. The high stability is avidity-driven and can be understood by considering that a spontaneous dissociation of AAV2 from a surrounding shell requires simultaneously breaking dozens of bonds formed between HS chains on the engulfing shell and the virus surface. The likelihood for such event to happen decreases exponentially with the number of HS bonds formed.

### Example 4: Efficiency of Encapsulation of Viruses/VLPs by DNA Origami Shells

### 1-Handle Design Development for Efficient Virus Trapping

To optimize and calibrate the density needed of our heparan sulfate derivatives, we exemplarily explored the trapping efficiencies of AAV2 with O half-shells using three different handle variants (FIG. 1G and FIG. 8a). The proximal handle (H1) was the shortest tested design and consisted of a DNA extension of 26 nucleotides, positioning the heparan sulfate modification in a proximal arrangement. The distal handle design (H2) included a single stranded extension of 20 thymidines (polyT extension), allowing for the heparan sulfate group to reach further from the origami surface and increase the chances of multivalent binding events. Finally, the branched design (H3) mimicked a branched polymer having two heparan sulfate modifications per handle unit, doubling the local heparan sulfate density.

The three handle designs were tested in parallel with O half-shells and excess of AAV2 particles. Samples were analyzed via negative stain TEM, where images were collected using an automated montage setup to minimize data collection bias. Particles were quantified blindly to estimate the number of full vs. empty shells for all three handle variants hybridized to heparan sulfate 3c. These experiments revealed that H1 was not as efficient for virus trapping as the longer and denser H2 and H3 handles. With H1, only 20% of O shells were occupied with AAV2, whereas with H₂ and H3 the trapping increased to 84% and 96%, respectively (FIG. 8b). The branched handle design (H3) hybridized to the heparan sulfate 18-mer variant 3c was used henceforth, unless otherwise stated.

### 2-Multi-Virus Trapping and Trapping of Different Virus Types in the Exact Same Shell Unit

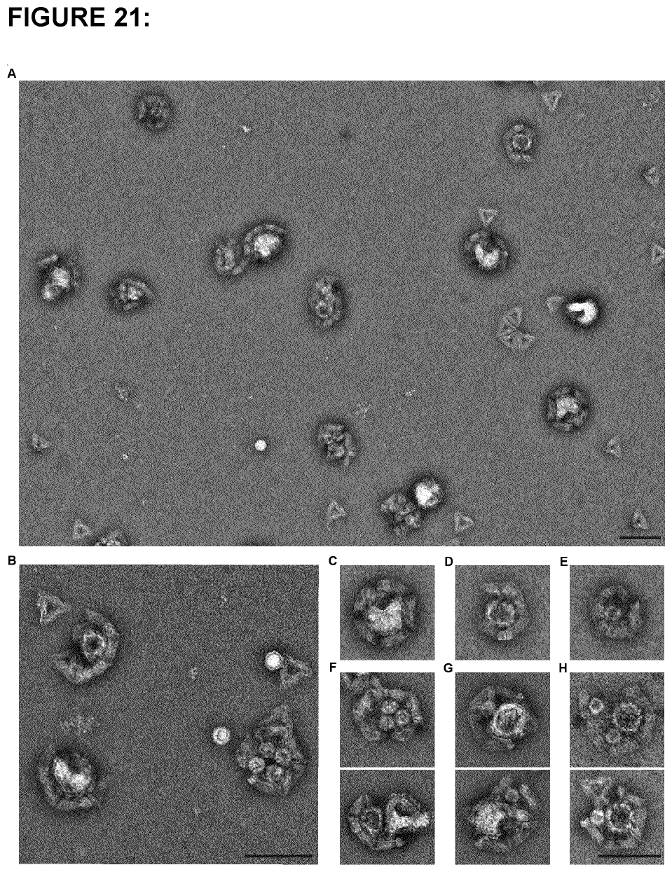

To test if our system could be used as a true broad-spectrum virus-trapping platform, heparan sulfate-modified T1 half-shells were challenged to trap a cocktail of viruses consisting of AAV2, HPV16 and Chikungunya particles. Negative stain TEM characterization of such sample revealed the trapping of all virus types present in the cocktail (FIG. 21). The field of view micrographs in FIGS. 21A and B exemplify the good performance of the system. Some shells were found to encapsulate individual viruses such as one Chikungunya particle (FIG. 21C), one HPV16 (FIG. 21D) and one AAV2 (FIG. 21E), but multiple particles were also trapped at once in the exact same shell unit as seen with several AAV2 (FIG. 21F top), HPV16-Chik (FIG. 21F bottom), AAV2-Chik (FIG. 21G), and AAV2-HPV16 (FIG. 21H).

### 3. Cooperative Shell Trapping of Virus Clusters

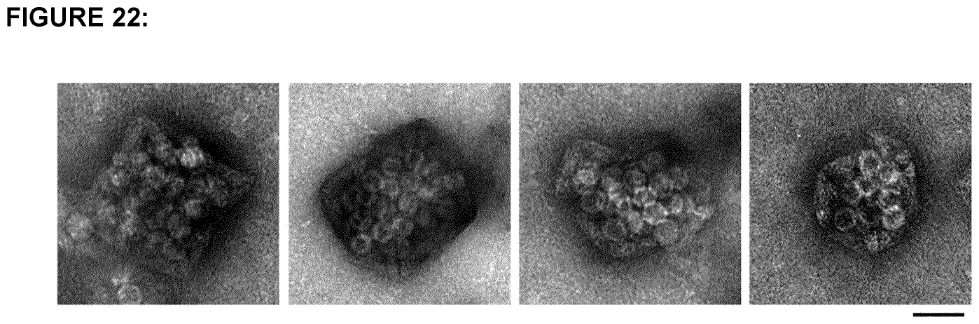

If the shell diameter was substantially larger than the target virus dimensions, we observed that multiple virus particles could be sequestered. Interestingly, multiple copies of HS-modified shells could partition over and cover the surface of AAV2 clusters (FIG. 22).

### SUMMARY

In conclusion, here we presented a viral trapping system that targets features of viruses that are conserved across many families through the usage of HS derivatives. Overall, we achieved encapsulation of nine different virus and VLP test samples, each representing a different viral family, and different sizes and surface complexities. Our modular shell system creates a locally curved environment within the cavity that enables highly multivalent binding, and that can be optimized according to size and ligand density/type to realize an irreversibly binding broad-spectrum antiviral platform. Our shells can flex and adapt to a certain degree to the shape of trapped virus particles, suggesting that the shell system can also adapt to pleomorphic virus particles.

We envision that our HS-modified DNA origami shells can act as a cellular surface decoy, sequestering the viruses and preventing interactions with cell surfaces, and thus reduce the effective viral load in acute infections. Testing the therapeutic potential of this system to reduce viral load in vivo remains an important task for the future. Beyond virus neutralization, our system may also serve as a sink for trapping associated viral proteins (FIG. 20), and other side products such as subviral particles that could potentially overwhelm the immune system (Zelikin, loc. cit.; Chai, N. et al., J. Virol. 82 (2008) 7812-7817). Overall, our results strongly indicate that our heparan sulfate-modified shell library has potential to become a relevant therapeutic platform to combat viral infections.

1. A DNA-based nanostructure,
wherein said DNA-based nanostructure is a shell comprises a cavity enclosed by said DNA-based nanostructure,
wherein said DNA-based nanostructure is formed by self-assembling DNA-based building blocks,
wherein each of said self-assembling DNA-based building blocks is formed by a single-stranded DNA template strand and a set of oligonucleotides complementary to said single-stranded DNA template,
wherein each of said oligonucleotides is either complementary to one contiguous DNA sequence stretch or to at least two non-contiguous DNA sequence stretches on said single-stranded DNA template,
wherein each of said self-assembling DNA-based building blocks is a triangular and/or a rectangular prismoid, particularly a triangular prismoid,
and wherein a subset of one or more of said oligonucleotides in one or more of said self-assembling DNA-based building blocks is/are each linked to a construct comprising at least one sulfonated or sulfated polysaccharide group pointing to the interior of said cavity, particularly a construct comprising one or two sulfonated or sulfated polysaccharide groups,
wherein each said construct comprises (i) a handle comprising at least one binding site for said sulfonated or sulfated polysaccharide group, and (ii) said sulfonated or sulfated polysaccharide group(s) bound to said handle; wherein said handle has a length corresponding to at least to the length of a single-stranded oligonucleotide comprising 30 nucleotides.

2. The DNA-based nanostructure according to claim 1, wherein each of said handles comprises two binding sites for said sulfonated or sulfated polysaccharide groups.

3. The DNA-based nanostructure according to claim 1, wherein each of said sulfonated or sulfated polysaccharide groups is independently selected from the group consisting of heparin, heparan sulfate, hybrid heparan sulfates, carrageenans, cellulose sulfate, and dextrin 2-sulfate.

4. The DNA-based nanostructure according to claim 3, wherein each of said sulfonated or sulfated polysaccharides is independently selected from a heparan sulfate and a hybrid heparan sulfate, in particular a heparan sulfate.

5. The DNA-based nanostructure according to claim 1, wherein one or more of said self-assembling DNA-based building blocks in said subset comprise n single-stranded oligonucleotides as said handles, wherein each handle is independently linked to at least one of said sulfonated or sulfated polysaccharide groups, wherein n is an integer independently selected from 1, 2, 3, 4, 5, 6, 7, 8, 9, 10, 11, and 12, particularly wherein n is 9.

6. The DNA-based nanostructure according to claim 1, wherein said handles are single-stranded oligonucleotides having a length of between 30 and 60 nucleotides, in particular between 40 and 55 nucleotides, more particularly between 45 and 50 nucleotides.

7. The DNA-based nanostructure according to claim 1, wherein each of said sulfonated or sulfated polysaccharide groups comprises an oligonucleotide having a sequence that is complementary to an oligonucleotide stretch comprised in said handles.

8. The DNA-based nanostructure of claim 1 comprising, a closed three-dimensional geometric shape, wherein the closed three-dimensional geometric shape is selected from the group consisting of: a sphere, a spherocylinder, a polyhedron, a tetrahedron, an octahedron and an icosahedron, wherein the DNA-based nanostructure is formed in situ from said self-assembling DNA-based building blocks in the presence of said viruses or viral particles to be encapsulated.

9. The DNA-based nanostructure of claim 1 comprising, a shell with an opening for accessing said cavity.

10. The DNA-based nanostructure of claim 1 comprising, a combination of a first and a second subshell, wherein each of said first and said second subshell comprises an opening to access a first and a second inner cavity, respectively, wherein said first and said second inner cavity together form said cavity, optionally wherein said first and said second subshell are connected by at least one linker.

11. The DNA-based nanostructure of claim 1 comprising, an icosahedral structure.

12. The DNA-based nanostructure of claim 11, wherein said DNA-based nanostructure is a DNA-based nanostructure formed by self-assembling DNA-based building blocks, wherein each of said self-assembling DNA-based building blocks is a triangular and/or a rectangular prismoid, optionally wherein each of said self-assembling DNA-based building blocks is a triangular prismoid.

13. The DNA-based nanostructure of claim 12,
wherein each said triangular and/or a rectangular prismoid is formed by m triangular, or rectangular, respectively, planes, wherein m is an integer independently selected from 4, 5, 6, 7 and 8, in particular independently selected from 5, 6 and 7, more particularly wherein said integer is 6, the three, or four, respectively, edges of each of said m planes are formed by n parallel stretches of DNA double helices, wherein n is an integer independently selected from 1, 2, 3, 4, 5 and 6 in particular independently selected from 2, 3, 4 and 5, more particularly independently selected from 3 and 4,
wherein each plane is connected to a plane above and/or a plane beyond said plane (i) by stacking interactions between the DNA double helices forming said planes, and (ii) partially by DNA stretches within said single-stranded DNA template and/or said oligonucleotides forming said DNA-based building block bridging at least two of said planes, and
wherein at least two of the three, or four, respectively, side trapezoids comprise a specific pattern of recesses and/or extrusions formed by missing or additional DNA double helical stretches for specific interaction with a complementary pattern on the side trapezoid of another one of said self-assembling DNA-based building blocks.

14. The DNA-based nanostructure of claim 1, wherein said DNA-based nanostructure is a half shell selected from the group consisting of:
(a) a half octahedron DNA-based nanostructure comprising T_octa self-assembling DNA-based building blocks, which consists of a set of four copies of a triangular frustum, wherein the base-pair stacking contacts on one of the triangular edges of the triangular frustum are inactivated by either strand shortening or by adding unpaired thymidines;
(b) a T=1 half shell comprising T1_pentamer_triangle self-assembling DNA-based building blocks, which consists of two sets of in each case five copies of two different triangular frusta, wherein the five copies of the first set form a closed pentamer, and the five copies of the second set dock onto the edges of said pentamer; and
(c) a “trap” T=1 half shell with a missing pentagon vertex comprising a combination of T1_pentamer_triangle and T1_ring_triangle self-assembling DNA-based building blocks, which consists of three sets of in each case five copies of three different triangular frusta, wherein the five copies of the first set form a closed pentamer, the five copies of the second set dock onto the edges of said pentamer the five copies of the second set dock onto the edges of said pentamer, and the five copies of the third set dock into the gaps between the five copies of said second set; and
(d) a T=3 icosahedral half shell comprising T3_6_triangle based self-assembling DNA-based building blocks, which consists of a total of 30 triangular subunits partitioned as five copies of six different full-size DNA triangle designs with specific edge docking rules.

15. The DNA-based nanostructure of claim 12, further comprising
(a) one or more types of DNA brick constructs, each type of such DNA brick constructs being characterized by one or more interaction sites for specific interaction by edge-to-edge stacking contacts with one or more complementary interaction sites present on the plane of a triangular or rectangular frustum on the outer surface of said DNA-based nanostructure, wherein said DNA brick constructs cover the free space between the three, or four, respectively, edges of said plane;
(b) one or more cross-linkages within one of said triangular or rectangular prismoids, and/or between two of said triangular and/or rectangular prismoids; and/or
(c) at least one moiety specifically interacting with said viruses or viral particles.

16. A composition comprising a DNA-based nanostructure according to claim 1 encapsulating one or more viruses or viral particles.

17. A method for encapsulating one or more viruses or viral particles, comprising the steps of: providing a DNA-based nanostructure according to claim 1, and contacting said DNA-based nanostructure with a medium comprising, or suspected to comprise, said viruses or viral particles.

18. The method of claim 17, wherein (i) a DNA-based half shell nanostructure comprising T_octa self-assembling DNA-based building blocks is selected for a virus of a size up to 50*50*50 nm³; (ii) the DNA-based half shell nanostructure comprising T1_pentamer_triangle self-assembling DNA-based building blocks is selected for a virus of a size between 15*15*15 and 100*100*100 nm³; (iii) the DNA-based half shell nanostructure comprising a combination of T1_pentamer_triangle and T1_ring_triangle self-assembling DNA-based building blocks is selected for a virus of a size between 15*15*15 and 100*100*100 nm³; and (iv) the DNA-based half shell nanostructure comprising T3_6_triangle self-assembling DNA-based building blocks is selected for a virus of a size of 50*50*50 nm³ or larger.
